(19) United States
(12) Patent Application Publication
(10) Pub. No.: US 2021/0402108 A1
(43) Pub. Date: Dec. 30, 2021

(54) SUPPLEMENTARY CONTINUOUS GAS SUPPLY SOURCE FOR DELIVERY TO SURGICAL CAVITIES

(71) Applicant: FISHER & PAYKEL HEALTHCARE LIMITED, Auckland (NZ)

(72) Inventors: Callum James Thomas Spence, Auckland (NZ); Pavlo Kokhanenko, Auckland (NZ); Zane Paul Gell, Auckland (NZ); Zach Jonathan Warner, Auckland (NZ); Benjamin Elliot Hardinge Pegman, Auckland (NZ); Abigail Sharmini Rajen Arulandu, Auckland (NZ); Rory Alexander Monro, Auckland (NZ); German Klink, Auckland (NZ); Gabor Papotti, Auckland (NZ)

(21) Appl. No.: 17/270,406

(22) PCT Filed: Aug. 23, 2019
(86) PCT No.: PCT/NZ2019/050105

§ 371 Date: Feb. 22, 2021

Related U.S. Application Data
(60) Provisional application No. 62/722,032, filed on Aug. 23, 2018.

(51) Int. Cl. A61M 13/00; A61B 17/34; A61M 16/16

(52) U.S. Cl. CPC A61M 13/006; A61B 17/3421; A61B 17/3474; A61M 16/16; A61M 2205/3368; A61M 2210/1021; A61M 2205/3344; A61M 2205/36; A61M 2205/7545; A61M 2210/1017; A61M 2205/3334

(57) ABSTRACT

Insufflation systems may provide a continuous flow of insufflation gas to a body cavity. The continuous flow may be directed over the lens of an endoscope received within a cannula to form a protective envelope around the lens and improve visibility. The continuous flow may be supplied by a pressurized gas source. The continuous flow line may be assembled in parallel to an insufflation line running through a standard insufflator configured to provide non-continuous gas flow to the body cavity. The lines may converge upstream of or at the cannula or the insufflation flow may be provided to a separate cannula. Continuous gas flow may be provided by recirculating gas from the body cavity through the cannula Continuous gas flow may be provided by storing gas from the non-continuous insufflation flow in an accumulator and releasing the gas during off phases of the insufflation flow.

### FIELD OF THE DISCLOSURE

The present disclosure relates to humidifier systems and components of humidifier systems for gases to be supplied to a patient, in particular to continuous supply systems for gases to be continuously flowed over medical instruments within surgical cavities to inhibit condensation and improve visual clarity.

### BACKGROUND

Various medical procedures require the provision of gases, typically carbon dioxide, to a patient during the medical procedure. For example, two general categories of medical procedures often require providing gases to a patient. These include closed type medical procedures and open type medical procedures.

In closed type medical procedures, an insufflator is arranged to deliver gases to a body cavity of the patient to inflate the body cavity and/or to resist collapse of the body cavity during the medical procedure. Examples of such medical procedures include laparoscopy and endoscopy, although an insufflator may be used with any other type of medical procedure as required. Endoscopic procedures enable a medical practitioner to visualize a body cavity by inserting an endoscope or the like through one or more natural openings, small puncture(s), or incision(s) to generate an image of the body cavity. In laparoscopy procedures, a medical practitioner typically inserts a medical instrument (e.g., a surgical element) through one or more natural openings, small puncture(s), or incision(s) to perform a medical procedure in the body cavity. In some cases, an initial endoscopic procedure may be carried out to assess the body cavity, and then a subsequent laparoscopy carried out to operate on the body cavity. Such procedures are widely used, for example, on the peritoneal cavity, or during a thoracoscopy, colonoscopy, gastroscopy or bronchoscopy.

In open type medical procedures, such as open surgeries for example, gases are used to fill a surgical cavity, with excess gases spilling outward from the opening. The gases can also be used to provide a layer of gases over exposed body parts for example, including internal body parts where there is no discernible cavity. For these procedures, rather than serving to inflate a cavity, the gases can be used to prevent or reduce desiccation and infection by covering exposed internal body parts with a layer of heated, humidified, sterile gases.

An apparatus for delivering gases during these medical procedures can include an insufflator arranged to be connected to a remote source of pressurized gases, such as a gases or fluid supply system in a hospital for example. The apparatus can be operative to control the pressure and/or flow of the gases from the gases source to a level suitable for delivery into the body cavity, usually via a cannula or needle connected to the apparatus and inserted into the body cavity, or via a diffuser arranged to diffuse gases over and into the wound or surgical cavity.

The internal body temperature of a human patient is typically around 37° C. It can be desirable to match the temperature of the gases delivered from the apparatus as closely as possible to the typical human body temperature. It can also be desirable to deliver gases above or below internal body temperature, such as, for example, 1° C., 2° C., 3° C., 4° C., 5° C., 6° C., 7° C., 8° C., 9° C., 10° C., or 15° C. above or below internal body temperature for example, or ranges including any two of the foregoing values. It can also be desirable to deliver gases at a desired fixed or variable humidity and/or a desired fixed or variable gas temperature. The gases at the desired gas temperature and/or humidity can be dry cold gas, dry hot gas, humidified cold gas, or humidified hot gas for example. Further, the gases delivered into the patient's body can be relatively dry, which can cause damage to the body cavity, including cell death or adhesions. In many cases, a humidifier is operatively coupled to the insufflator. A controller of the apparatus can energize a heater of the humidifier located in the gases flow path to deliver humidification fluid, such as water for example, vapor to the gases stream prior to entering the patient's body cavity.

The humidified gas can be delivered to the patient via further tubing which may also be heated. The insufflator and humidifier can be located in separate housings that are connected together via suitable tubing and/or electrical connections, or located in a common housing arranged to be connected to a remote gas supply via suitable tubing.

### SUMMARY

During endoscopic procedures, such as laparoscopy, thoracoscopy, colonoscopy, sigmoidoscopy, gastroscopy, bronchoscopy, etc., an endoscope or another medical instrument may be inserted through a surgical incision or a natural body opening into an enclosed body cavity. For example, during laparoscopic surgery an endoscope may be surgically inserted through a small incision through the peritoneum into the abdominal cavity. The endoscope may be inserted through a cannula or similar structure which may be configured to receive the endoscope and/or other surgical tools and establish a pathway between the body cavity and the ambient environment which allows a surgeon or physician to operate internally within the body cavity through the cannula. The endoscope or another visualization tools may be needed by the surgeon to visualize the inside of the body cavity and perform a procedure inside the body cavity such as a surgical operation for example. During such endoscopic procedures, it is common to insufflate the body cavity with a fluid, including gases (e.g., air or carbon dioxide) or liquids. In some cases, while the term gas or gases can be used to refer to the fluid that is inserted through the cannula and/or into the cavity as described herein, it is understood that any fluid, including any gas or liquid, can be used to expand or increase the volume of the body cavity or perform other functions including those disclosed herein. The expansion of the body cavity may provide for additional work space for the surgeon and/or provide better visibility of target structures within the body cavity. Insufflators are commonly used pieces of equipment for establishing and maintaining an insufflated environment. Insufflators are generally configured to provide non-continuous or pulsatile flow comprising phases of positive pressure for inducing the flow of an insufflation gas into the body cavity to expand the cavity and phases of no gas flow (off phases) for maintaining the pressure and/or volume of the body cavity below a threshold pressure and/or volume. Insufflation gas introduced into the body cavity may gradually leak from the body cavity through an imperfect seal between the body cavity and the cannula and/or through backflow through the insufflation line. Accordingly, insufflators may switch between phases or pulses of positive pressure and no pressure to attempt to maintain the body cavity at a desired pressure and/or volume. The insufflator may comprise a pressure sensor for monitoring pressure within the body cavity and may be configured to switch between phases of positive pressure and no pressure (or between phases of higher and lower pressure) to maintain the desired pressure and/or volume. Pressure may also be released through venting features inserted into the body cavity or attached to a cannula or may be actively released through suctioning or other means. Ventilation may advantageously remove smoke generated from electrosurgery, electrocautery, laser cutting or cauterizing, or other types of energy, from the body cavity, which can prevent or at least reduce condensation and/or fogging. Condensation can occur on various surfaces on a medical instrument. When condensation forms on a viewing surface of a medical instrument, this is observed as a fogging effect which manifests as an impairment of visibility through a lens or any other viewing surface of a medical instrument (such as, for example, a minor or transparent or translucent window). When condensation forms on various surfaces of a medical instrument, the condensation can coalesce into water droplets. This can occur directly on the viewing surface or other surfaces which can then migrate to or be deposited on the viewing surface. Accordingly, as used herein condensation and/or fogging means condensation generally and in some instances, specifically with respect to condensation on a viewing surface (i.e. fogging). While the application of fluids, for example gases, surrounding the medical instrument is described as potentially important for visualization of the surgical area by a scope or other visualization device, the application of fluids as described herein can be used in applications such as electrocautery tools, graspers, and other instruments.

The embodiments of surgical systems disclosed herein may provide a secondary or supplementary source of gas flow into the body cavity, such as through a cannula for example. The surgical system may be an insufflation system. Some embodiments of surgical systems disclosed herein are generally configured to provide a continuous flow of gas to the cannula to improve visualization through the endoscope. The continuous flow of gas may supplement or may supplant the flow of gas provided by an insufflator. In some embodiments, the continuous flow may be provided by a pressurized gas source or may be established by recirculation of the gas within the body cavity. The provision of a supplemental and continuous flow of gas may cause an insufflator to be used only sparingly or not at all. Nonetheless an insufflator may be used in combination with the supplemental flow for safety precautions. The term “continuous gas flow” means that a positive amount of gas flow is delivered into the body cavity. The positive gas flow may be steady (a flat or constant flow rate), pulsatile, or irregularly continuous (random). Positive gas flow can result in establishing and maintaining a positive pressure gradient across a cannula through which the flow is delivered, such that the pressure is higher at the inlet of the cannula than at the outlet of the cannula. The positive pressure gradient can ensure that gases move in a direction toward the body cavity and that gases do not flow back up the cannula. The positive pressure gradient inhibits or prevents fogging of an endoscope and/or other medical instruments inserted into the cannula and/or prevents or inhibits gases and/or smoke from the body cavity form coming into contact with a lens of the endoscope. The gas flow provided may also pressurize the body cavity and inflate the body cavity to provide a workspace for a surgeon.

In some embodiments, the surgical systems, including insufflation systems disclosed herein may employ standard surgical cannulas configured to receive the endoscope and/or other surgical tools (e.g., through a working channel of the endoscope). In some embodiments, the surgical system may use a cannula which is specifically configured to direct air flow across or over a lens of the endoscope (or any other medical instrument) to form a continuous flow envelope of gas around the lens and possibly other portions of the endoscope. The cannula may precisely control the shape of the envelope around the endoscope. For example, the cannula can include a guiding element that guides an endoscope inserted into the cannula to be held substantially concentrically within a receiving lumen of the cannula. The guiding element may prevent the endoscope from resting against a sidewall of the cannula when inserted into the receiving lumen. The cannula may hold the endoscope in a concentric configuration and direct gas flow evenly around the outer diameter of the endoscope to form a substantially symmetric gas envelope which fully encloses the lens of the endoscope. Such a configuration may cause the gas envelope to extend further along a length of the endoscope shaft according to the Coanda effect, which causes a fluid jet stream to remain attached to a surface, including but not limited to a convex surface. The Coanda effect can cause the gases of the continuous gas flow to flow distally beyond the end of the endoscope. The gases delivered through the cannula and around (e.g., concentrically) the endoscope can cause the gases to closely hug or enfold the endoscope. The cannula can be fluidly coupled to the supplementary gases delivery system. The supplementary gases delivery system can be configured to deliver a continuous gas flow (i.e. a positive gases flow) as described elsewhere herein. The delivery of gas may follow a steady, pulsatile, or random pattern. The continuous gases flow can ensure a positive gases flow within the cannula and around the scope. The continuous gases flow can help create a continuous protection envelope. The positive pressure prevents the envelope from breaking down since gases do not flow back up the cannula. The cannula can be single use (disposable) or reusable. Alternatively, parts of the cannula can be single use (disposable) or reusable. The cannula may be made of materials that are biocompatible and/or sterilizable. In the present disclosure, features of the different examples of cannulas can be incorporated into or combined with one another.

Any of the supplementary gas supply systems discussed herein may be used in combination with a cannula configured to direct gas flow, such as a cannula comprising a guiding element as described elsewhere herein for example. A cannula used to deliver the continuous gas flow may include one or more heating elements disposed in a portion of the cannula to heat the gases directly and/or to heat the cannula to transfer heat to the gases flowing through the cannula. The heating elements may be configured additionally or alternatively to heat the endoscope received into the cannula directly and/or to heat the cannula to transfer heat to the endoscope. The cannula may comprise a venting pathway formed within a body of the cannula. One or more filter elements may be disposed in the venting pathway. The one or more heating elements may be disposed on or within the venting pathway. The one or more heating elements may further extend through or about the one or more filter elements. Alternatively, a separate heating element may be disposed on, within, or through the filter elements.

The cannula may be designed for use with a specific brand and/or size of endoscope and/or may be adaptable to use with any standard endoscope. The envelope may extend around additional surgical tools inserted through the cannula and/or may be used on devices or tools other than an endoscope. The envelope of continuously flowing gas over the lens of the endoscope may improve optical clarity and help maintain a clear field of vision for a surgeon. For instance, the envelope may prevent or inhibit any uncontrolled gases (e.g., the ambient gas inside the body cavity) from coming into contact with the endoscope lens. The envelope may prevent or inhibit humid air from condensing on the endoscope lens and reducing visibility. The envelope may prevent or inhibit smoke generated from electrosurgery, electrocautery, ultrasound, laser cutting or cauterising, fog in the body cavity, fat films, and/or other debris from becoming deposited on the endoscope lens and/or inhibiting the field of view of the endoscope. The envelope may create a protective gas layer around the lens of the endoscope and/or deflect or redirect particles in the ambient environment away from the lens of the endoscope. The condition of the gas envelope (e.g., the temperature and/or the humidity) can also be controlled. By controlling the condition of the continuous gas flow envelope, the envelope may be maintained above the dew point of the gas to prevent and/or at least reduce condensation on the endoscope. The surgical system can modulate the pressure and/or flow rate of the continuous gas flow to manipulate the dew point.

In one aspect of the present disclosure, disclosed herein is a surgical system for delivering gases to a surgical cavity. The surgical system may be an insufflation system. The surgical system includes a supplementary gases module having an inlet configured to be placed in fluid communication with an upstream pressurized gas source and an outlet configured to be placed in fluid communication with a downstream surgical cannula to establish at least one gas flow pathway from the pressurized gas source to the surgical cannula. The supplementary gases module comprises at least one pressure regulator configured to establish a pressure drop between the pressurized gas source and the surgical cannula. The surgical system further includes the surgical cannula. The surgical cannula has a proximal end configured to be positioned outside of a body cavity and a distal end configured to be inserted into the body cavity. The surgical cannula has a medical instrument lumen configured to receive a medical instrument such that the medical instrument may extend from an ambient environment outside of the body cavity through the medical instrument lumen into the body cavity. The surgical cannula has an endoscope lumen configured to receive an endoscope such that the endoscope may extend from an ambient environment outside of the body cavity through the endoscope lumen into the body cavity. The surgical cannula has an inlet gas flow pathway configured to be placed in fluid communication with the outlet of the supplementary gases module, the inlet gas flow pathway intersects the medical instrument lumen such that a continuous flow of gas from the pressurized gas source is configured to be flowed over a distal end of the medical instrument when received in the medical instrument lumen. The surgical cannula has an inlet gas flow pathway configured to be placed in fluid communication with the outlet of the supplementary gases module, the inlet gas flow pathway intersects the endoscope lumen such that a continuous flow of gas from the pressurized gas source is configured to be flowed over a distal end of the endoscope when received in the endoscope lumen.

In some configurations, the supplementary gases module includes a flow control element configured to induce and/or inhibit the flow of gas through a gas flow line extending from the upstream pressurized gas source to the downstream surgical cannula.

In some configurations, the flow control element has a motorized fan.

In some configurations, the supplementary gases module includes one or more sensors configured to sense a parameter of the gas flow downstream of the pressure regulator.

In some configurations, at least one of the one or more sensors is a pressure sensor, a flow rate sensor, a humidity sensor, or a temperature sensor.

In some configurations, the supplementary gases module is configured to modulate the flow rate of gas through the supplementary gases module in response a reading determined by at least one of the one or more sensors.

In some configurations, the surgical system includes a humidifier having an inlet and an outlet positioned operably between the pressure regulator and the surgical cannula and configured to increase the humidity of the continuous gas flow. The surgical system may be an insufflation system.

In some configurations, the humidifier is part of the supplementary gases module.

In some configurations, the inlet of the humidifier is configured to be placed in fluid communication with an outlet of the supplementary gases module and the outlet of the humidifier is configured to be placed in fluid communication with inlet gas flow pathway of the surgical cannula.

In some configurations, the supplementary gases module includes a housing enclosing components of the supplementary gases module.

In some configurations, the supplementary gases module includes a gas storage chamber positioned operably downstream of the pressure regulator.

In some configurations, the supplementary gases module is configured to be positioned vertically above the surgical cannula such that head pressure of gas stored in the gas storage chamber is configured to drive gas flow downstream to the surgical cannula.

In some configurations, the surgical system includes an insufflator comprising an inlet configured to be placed in fluid communication with the pressurized gas source and an outlet configured to be placed in fluid communication with a downstream insufflation cannula. The surgical system may be an insufflation system. The insufflator can be configured to provide a non-continuous flow of gas to the insufflation cannula and to be arranged in parallel with the supplementary gases module between the pressurized gas source and the body cavity.

In some configurations, the insufflator includes a pressure regulator configured to establish a pressure drop between the pressurized gas source and the insufflation cannula.

In some configurations, the insufflator includes a pressure sensor configured to measure pressure within the body cavity, the insufflator being configured to initiate gas flow to the insufflation cannula or increase the flow rate of gas flow to the insufflation cannula when the measured pressure falls below a predetermined threshold pressure.

In some configurations, the surgical system includes a humidifier having an inlet and an outlet positioned operably between the insufflator and the insufflation cannula and configured to increase the humidity of the non-continuous gas flow. The surgical system may be an insufflation system.

In some configurations, the inlet of the humidifier is configured to be placed in fluid communication with an outlet of the insufflator and an outlet of the supplementary gases module.

In some configurations, the surgical system includes a Y-shaped connector placing the inlet of the humidifier in fluid communication with an outlet of the insufflator and an outlet of the supplementary gases module. The surgical system may be an insufflation system.

In some configurations, the humidifier has a second inlet configured to be placed in fluid communication with an outlet of the supplementary gases module.

In some configurations, the insufflation cannula is the surgical cannula.

In some configurations, the surgical system includes a switch valve having a first inlet positioned in series with an outlet of the supplementary gases module, a second inlet positioned in series with an outlet of the insufflator, and an outlet positioned in series with the inlet gas flow pathway of the surgical cannula. The switch valve can be closed to gas flow from the supplementary gases module when gas is flowing from the insufflator and open to gas flow from the supplementary gases module when gas is not flowing from the insufflator. The surgical system may be an insufflation system.

In some configurations, the surgical system includes a humidifier configured to be positioned in series with the switch valve and the surgical cannula. The surgical system may be an insufflation system.

In some configurations, the surgical cannula is configured to be coupled to two separate gas flow conduits and to fluidly connect the two separate gas flow conduits to the inlet gas flow pathway.

In some configurations, the supplementary gases module is an insufflator configured to provide a non-continuous flow of gas to the outlet of the supplementary gases module.

In some configurations, the surgical system includes an accumulator configured to be positioned in series or in a parallel with the at least one gas flow pathway operably downstream of the supplementary gases module. The accumulator can have an expandable volume configured to expand when gas flow is delivered to the accumulator from an upstream direction to store a portion of the gas delivered to the accumulator and to contract when gas flow is not delivered to the accumulator from an upstream direction to release at least some of the gas stored by the accumulator downstream through the at least one gas flow pathway. The surgical system may be an insufflation system.

In some configurations, the accumulator is used with an insufflator.

In some configurations, the accumulator acts as a supplementary gas supply and releases gas when pressure in the body cavity (e.g., the pneumoperitoneum) drops below a threshold pressure.

In some configurations, the threshold pressure below which the accumulator releases gas is set such that a positive pressure gradient is maintained across the surgical cannula.

In some configurations, the surgical system may comprise a plurality of accumulators (e.g., two, three, four, or more). The surgical system may be an insufflation system.

In some configurations, the one or more accumulators are in fluid communication with the outlet of the supplementary gases module and the outlet of an insufflator. The one or more accumulators may be configured to release a flow of gases if the pressure in the body cavity drops below a threshold pressure.

In some configurations, the surgical system includes a one-way valve positioned upstream of the accumulator and configured to allow gas flow only in a downstream direction such that stored gas released from the accumulator cannot travel in an upstream direction to the supplementary gases module. The surgical system may be an insufflation system.

In some configurations, the accumulator has a flexible membrane configured to expand to increase the volume of the expandable volume and to contract to decrease the volume of the expandable volume.

In some configurations, the accumulator has an internal volume partitioned by a moveable fluid seal between the expandable volume in series with the at least one gas flow pathway and a second volume not in series with the at least one gas flow pathway. The moveable fluid seal can be biased to place the expandable volume in a relatively unexpanded state.

In some configurations, the moveable fluid seal is biased by a compressible piston.

In some configurations, the moveable fluid seal is biased by a compressible spring.

In some configurations, the accumulator is coupled to an inlet of the surgical cannula upstream of the inlet gas flow pathway of the surgical cannula.

In some configurations, the accumulator is coupled to an outlet of a humidifier.

In some configurations, the accumulator is coupled to or incorporated in the surgical cannula and operably positioned between an inlet of the cannula and the intersection of the inlet gas flow pathway with the medical instrument lumen. In some configurations, the accumulator is coupled to or incorporated in the surgical cannula and operably positioned between an inlet of the cannula and the intersection of the inlet gas flow pathway with the endoscope lumen.

In some configurations, the accumulator is enclosed within a rigid body of the surgical cannula.

In some configurations, the accumulator extends from a rigid body of the surgical cannula.

In some configurations, the accumulator comprises a generally toroidal configuration and is configured to annularly surround the medical instrument lumen. In some configurations, the accumulator comprises a generally toroidal configuration and is configured to annularly surround the endoscope lumen.

In some configurations, the accumulator is positioned in parallel with the at least one gas flow pathway and the system further includes a valve switch downstream of the accumulator. The valve switch can be configured to allow gas to flow downstream from the accumulator to the surgical cannula when gas flow is not being delivered to the accumulator and not when gas flow is being delivered to the accumulator. The valve switch can be configured to allow gas flow to be delivered through the at least one gas flow pathway in parallel to the accumulator when gas flow is being delivered to the accumulator and not when gas flow is not being delivered to the accumulator.

In some configurations, the surgical system includes a feedback line arranged in parallel with the at least one gas flow pathway configured to establish a return gas flow pathway between the body cavity and an insufflator in series with the at least one gas flow pathway such that the insufflator may continuously monitor the pressure of the body cavity. The surgical system may be an insufflation system. The feedback line can include a one-way valve configured to place the feedback line in fluid communication with the insufflator only when gas flow is not being delivered in a downstream direction from the insufflator.

In some configurations, the feedback line includes a feedback modifier configured to artificially lower a pressure reading of the insufflator in order to induce the insufflator to provide continuous downstream gas flow to the surgical cannula.

In some configurations, the feedback line is coupled to the surgical cannula.

In some configurations, the surgical cannula includes a feedback gas flow pathway distinct from the inlet gas flow pathway and the medical instrument lumen and having an opening configured to be placed in fluid communication with the body cavity. In some configurations, the surgical cannula includes a feedback gas flow pathway distinct from the inlet gas flow pathway and the endoscope lumen and having an opening configured to be placed in fluid communication with the body cavity.

In some configurations, the opening to the feedback gas flow pathway is disposed on a side of an outer diameter of a shaft of the surgical cannula configured to extend into the body cavity.

In some configurations, the surgical system includes a one-way backflow valve operably positioned along the gas flow pathway upstream of the medical instrument lumen to prevent gas flow from entering the at least one gas flow pathway from a location downstream of the backflow valve. The surgical system may be an insufflation system. In some configurations, the surgical system or insufflation system includes a one-way backflow valve operably positioned along the gas flow pathway upstream of the endoscope lumen to prevent gas flow from entering the at least one gas flow pathway from a location downstream of the backflow valve.

In some configurations, the surgical system includes a recirculation cannula and a recirculation gas flow pathway. The surgical system may be an insufflation system. The recirculation cannula can have a proximal end configured to be positioned outside of the body cavity, a distal end configured to be inserted into the body cavity, and an intake gas flow pathway configured to allow gas to enter the recirculation cannula from the body cavity. The recirculation gas flow pathway can connect the intake gas flow pathway of the recirculation cannula and the medical instrument lumen of the surgical cannula. The recirculation gas flow pathway can connect the intake gas flow pathway of the recirculation cannula and the endoscope lumen of the surgical cannula.

In some configurations, the recirculation gas flow pathway includes a filter configured to filter the gas traveling through the recirculation gas flow pathway before it enters the medical instrument lumen. In some configurations, the recirculation gas flow pathway includes a filter configured to filter the gas traveling through the recirculation gas flow pathway before it enters the endoscope lumen.

In some configurations, the recirculation gas flow pathway includes a material barrier between the recirculation gas flow pathway and the ambient environment configured to allow humidification fluid vapor to diffuse through the barrier into the ambient environment in order to remove condensation from the recirculation gas flow pathway. In some configurations, the recirculation gas flow pathway includes a material barrier between the recirculation gas flow pathway and the ambient environment configured to allow water vapor to diffuse through the barrier into the ambient environment in order to remove condensation from the recirculation gas flow pathway.

In some configurations, the recirculation gas flow pathway comprises an electrically driven suction unit configured to drive gas flow through the recirculation gas flow pathway.

In some configurations, the electrically driven suction unit is enclosed within a rigid body of the recirculation cannula.

In some configurations, the electrically driven suction unit is physically coupled to the recirculation cannula.

In some configurations, the electrically driven suction unit is operably coupled to the recirculation cannula by fluid channels forming a closed loop with the recirculation cannula.

In some configurations, the fluid channels are arranged concentrically within a single conduit.

In some configurations, the surgical cannula is the recirculation cannula.

In some configurations, the recirculation gas flow pathway includes a fan configured to drive gas flow through the recirculation gas flow pathway. The fan can be coupled to a turbine outside of the recirculation gas flow pathway such that rotation of the turbine causes rotation of the fan. The turbine can be driven by gas flow across the turbine.

In some configurations, the gas flow that drives the turbine is gas flow through the at least one gas flow pathway driven by the pressurized gas source. In some configurations, the gas flow that drives the turbine is gas flow through an insufflation line configured to deliver insufflation gas from a pressurized gas source to the body cavity.

In some configurations, the recirculation gas flow pathway converges with the at least one gas flow pathway upstream of the surgical cannula.

In some configurations, the recirculation gas flow pathway converges with a conduit of the at least one gas flow pathway at a point where the conduit has a reduced cross-sectional area over an intermediate length of the conduit. The reduced cross-sectional area may be reduced sufficiently enough to create a pressure drop by the Venturi effect within the conduit over the intermediate length generating a pressure gradient configured to drive suction of gas from the recirculation gas flow pathway into the conduit.

In some configurations, the recirculation gas flow pathway converges with the at least one gas flow pathway upstream of a humidifier positioned within the at least one gas flow pathway.

In some configurations, the recirculation gas flow pathway is coupled to an inlet of a humidifier positioned within the at least one gas flow pathway.

In some configurations, the pressure regulator has an elongated gas flow pathway configured to exert sufficient friction on gas travelling through the pressure regulator to establish the pressure drop.

In some configurations, the elongated gas flow pathway is disposed across a two-dimensional area in a manner that fills the entire two-dimensional area, the two-dimensional area having a surface area that is significantly larger than the cross-sectional area of the elongated pathway.

In some configurations, the elongated gas flow pathway has a spiral configuration.

In some configurations, the elongated gas flow pathway has a raster-pattern configuration.

In some configurations, the pressure regulator is formed within a humidifier and the elongated gas flow pathway extends over a volume of humidification fluid stored in the humidifier. In some configurations, the pressure regulator is formed within a humidifier and the elongated gas flow pathway extends over a volume of water stored in the humidifier.

In some configurations, the pressure regulator is configured to float within a chamber of a humidifier, the volume of humidification fluid forming a floor of the elongated gas flow pathway. In some configurations, the pressure regulator is configured to float within a chamber of a humidifier, the volume of water forming a floor of the elongated gas flow pathway.

In some configurations, the pressure regulator includes an orifice plate having a plurality of orifices reducing the available cross-sectional area through which gas may flow through the at least one gas flow pathway to establish the pressure drop.

In some configurations, the pressure regulator has a conduit having a restricted diameter over a length sufficient to establish the pressure drop.

In some configurations, the pressure regulator has a pressure release feature configured to release pressure from the at least one gas flow pathway to the ambient environment if the pressure within the pressure regulator exceeds a threshold pressure.

In some configurations, the pressure release feature includes a pressure release valve in fluid communication with the at least one gas flow pathway and extending downward into a liquid bath. A pressure of the gas within the at least one gas flow pathway above the threshold pressure may force the liquid level within the pressure release valve low enough such that gas may escape through the bottom of the pressure release valve into the liquid bath to be released to the ambient environment.

In some configurations, the pressure release feature includes a small gap or aperture in a barrier between the at least one gas flow pathway and the ambient environment disposed on a portion of the at least one gas flow pathway having a reduced diameter. The gap can be configured in size to prevent gas flow from the at least one gas flow pathway escaping to the ambient environment unless the pressure of the gas exceeds the threshold pressure.

In some configurations, the surgical system includes a pressure release cannula configured to vent pressure from the body cavity to the ambient environment. The surgical system may be an insufflation system.

In some configurations, the pressure release cannula is configured to vent gas at a constant gas flow rate.

In some configurations, the pressure release cannula has an adjustment feature configured to adjust the flow rate of gas vented to the ambient environment.

In some configurations, the surgical cannula is the pressure release cannula.

In some configurations, the surgical cannula is configured to be heated to regulate the temperature of the continuous gas flow.

In some configurations, the surgical cannula is configured to direct gas flow across a lens of the medical instrument to form a continuous flow gas envelope around the lens. In some configurations, the surgical cannula is configured to direct gas flow across a lens of the endoscope to form a continuous flow gas envelope around the lens.

In some configurations, the medical instrument is configured to be held concentrically centered within the medical instrument lumen such that gas flow is configured to be delivered to the medical instrument lumen uniformly around the outer diameter of the medical instrument. In some configurations, the endoscope is configured to be held concentrically centered within the endoscope lumen such that gas flow is configured to be delivered to the endoscope lumen uniformly around the outer diameter of the endoscope.

In some configurations, the medical instrument is configured to form a fluid seal with the surgical cannula to prevent significant release of gas from the inlet gas flow pathway or from the body cavity to be released to the ambient environment through the medical instrument lumen. In some configurations, the endoscope is configured to form a fluid seal with the surgical cannula to prevent significant release of gas from the inlet gas flow pathway or from the body cavity to be released to the ambient environment through the endoscope lumen.

In some configurations, the surgical cannula comprises a pointed distal end configured to facilitate insertion of the surgical cannula into the body cavity.

In some configurations, the surgical system includes the pressurized gas source. The surgical system may be an insufflation system.

In some configurations, the surgical system is configured to pressurize the body cavity and inflate the body cavity to provide a workspace for surgeons. The surgical system may be an insufflation system.

In another aspect of the present disclosure, disclosed herein is a surgical system for delivering gases to a surgical cavity. The surgical system may be an insufflation system. The surgical system has a surgical cannula having a proximal end configured to be positioned outside of a body cavity and a distal end configured to be inserted into the body cavity. The surgical cannula has a medical instrument lumen configured to receive a medical instrument such that the medical instrument may extend from an ambient environment outside of the body cavity through the medical instrument lumen into the body cavity. The surgical cannula has a medical instrument lumen configured to receive an endoscope such that the endoscope may extend from an ambient environment outside of the body cavity through the endoscope lumen into the body cavity. The surgical cannula has an inlet gas flow pathway configured to be placed in fluid communication with a pressurized gas source. The inlet gas flow pathway intersects the medical instrument lumen such that a continuous flow of gas from the pressurized gas source is configured to be flowed over a distal end of the medical instrument when received in the medical instrument lumen. The inlet gas flow pathway intersects the endoscope lumen such that a continuous flow of gas from the pressurized gas source is configured to be flowed over a distal end of the endoscope when received in the endoscope lumen. The surgical cannula has a pressure regulator enclosed within the surgical cannula and configured to establish a pressure drop between the pressurized gas source and the medical instrument lumen. The surgical cannula has a pressure regulator enclosed within the surgical cannula and configured to establish a pressure drop between the pressurized gas source and the endoscope lumen.

In some configurations, the surgical system includes an insufflator configured to provide a non-continuous flow of gas to an insufflation cannula and to be arranged in parallel with a gas flow pathway extending from the pressurized gas flow source to the surgical cannula. The surgical system may be an insufflation system.

In some configurations, the insufflator is coupled to the same pressurized gas source as the surgical cannula.

In another aspect of the present disclosure, disclosed herein is a surgical system for delivering gases to a surgical cavity. The surgical system may be an insufflation system. The surgical system includes a surgical cannula, a recirculation cannula, and a recirculation gas flow pathway. The surgical cannula has a proximal end configured to be positioned outside of a body cavity and a distal end configured to be inserted into the body cavity. The surgical cannula has a medical instrument lumen configured to receive a medical instrument such that the medical instrument may extend from an ambient environment outside of the body cavity through the medical instrument lumen into the body cavity. The surgical cannula has an endoscope lumen configured to receive an endoscope such that the endoscope may extend from an ambient environment outside of the body cavity through the endoscope lumen into the body cavity. The recirculation cannula has a proximal end configured to be positioned outside of the body cavity, a distal end configured to be inserted into the body cavity, and an intake gas flow pathway configured to allow gas to enter the recirculation cannula from the body cavity. The recirculation gas flow pathway connecting the intake gas flow pathway of the recirculation cannula and the medical instrument lumen of the surgical cannula, such that a continuous flow of gas is configured to be flowed over a distal end of the medical instrument when received in the medical instrument lumen. The recirculation gas flow pathway connecting the intake gas flow pathway of the recirculation cannula and the endoscope lumen of the surgical cannula, such that a continuous flow of gas is configured to be flowed over a distal end of the endoscope when received in the endoscope lumen.

In some configurations, the recirculation gas flow pathway includes a filter configured to filter the gas traveling through the recirculation gas flow pathway before it enters the medical instrument lumen. In some configurations, the recirculation gas flow pathway includes a filter configured to filter the gas traveling through the recirculation gas flow pathway before it enters the endoscope lumen.

In some configurations, the recirculation gas flow pathway includes a material barrier between the recirculation gas flow pathway and the ambient environment configured to allow humidification fluid vapor to diffuse through the barrier into the ambient environment in order to remove condensation from the recirculation gas flow pathway. In some configurations, the recirculation gas flow pathway includes a material barrier between the recirculation gas flow pathway and the ambient environment configured to allow water vapor to diffuse through the barrier into the ambient environment in order to remove condensation from the recirculation gas flow pathway.

In some configurations, the recirculation gas flow pathway includes an electrically driven suction unit configured to drive gas flow through the recirculation gas flow pathway.

In some configurations, the electrically driven suction unit is enclosed within a rigid body of the recirculation cannula.

In some configurations, the electrically driven suction unit is physically coupled to the recirculation cannula.

In some configurations, the electrically driven suction unit is operably coupled to the recirculation cannula by fluid channels forming a closed loop with the recirculation cannula.

In some configurations, the fluid channels are arranged concentrically within a single conduit.

In some configurations, the recirculation gas flow pathway includes a fan configured to drive gas flow through the recirculation gas flow pathway. The fan can be coupled to a turbine outside of the recirculation gas flow pathway such that rotation of the turbine causes rotation of the fan. The turbine can be driven by gas flow across the turbine.

In some configurations, the gas flow that drives the turbine is gas flow through an insufflation line configured to deliver insufflation gas from a pressurized gas source to the body cavity.

In some configurations, the surgical system includes an insufflator configured to provide a non-continuous flow of gas to an insufflation cannula.

In another aspect, the present disclosure is directed to a surgical system for providing a gases flow to a body cavity. The surgical system may be an insufflation system. The surgical system includes an insufflator device, a first delivery conduit, a first cannula, and a supplementary gas system. The first conduit fluidly couples the insufflator device to the first cannula. The insufflator is configured to generate a gases flow of insufflation gases, the insufflation gases being delivered to the first cannula via the first conduit and the insufflation gases being introduced into the body cavity through the first cannula. The supplementary gas system is configured to deliver an additional flow of gases to the body cavity, the additional flow of gases being in addition to the insufflation gases from the insufflator.

In some aspects, the supplementary gas system includes a supplementary gases flow module, a second delivery conduit, and a second cannula. In some configurations, the supplementary gas system has a supplementary gases flow module, a humidifier, a second delivery conduit, and a second cannula. The humidifier can be arranged in fluid communication and downstream of the supplementary gases flow module and the humidifier can be configured to receive supplementary gases from the supplementary gases module and to humidify said supplementary gases. The second delivery conduit can be in fluid communication with the second cannula, the second cannula receiving humidified supplementary gases from the humidifier via the second delivery conduit. The supplementary gases can be provided into the body cavity via the second cannula, such that the body cavity is supplied with heated and humidified gases.

In some configurations, the supplementary gases flow module is configured to provide a continuous gases flow of supplementary gases that is heated, humidified and delivered to the body cavity via the second cannula.

In some configurations, the supplementary gases flow module provides a continuous flow of supplementary gases to the body cavity through the second cannula.

In some configurations, the supplementary gas system (i.e. the supplementary gases flow module, humidifier, tube, and second cannula) is configured to supplement a gas flow into the body cavity in order attempt to maintain a constant pressure within the body cavity.

In some configurations, the supplementary gas system provides a continuous flow of gases that may have a pulsatile, steady, or random flow pattern, wherein the supplementary gases are always greater than 0 L/min.

In some configurations, the supplementary gas system is configured to deliver supplementary gases through the second cannula such that a positive pressure gradient is maintained across the second cannula.

In some configurations, the positive pressure gradient is maintained by delivering continuous flow such that the pressure at the inlet of the cannula is greater than at the outlet of the cannula such that gases always flow into the body cavity.

In some configurations, the second cannula may include one or more guiding features configured to orient a medical instrument within the cannula such that the medical instrument is substantially concentric within the cannula. In some configurations, the second cannula may include one or more guiding features configured to orient an endoscope within the cannula such that the endoscope is substantially concentric within the cannula.

In some configurations, the guiding features are configured to guide the scope within the cannula such that the supplementary gases flow surrounds the medical instrument or envelopes the medical instrument, and a protection envelope of heated humidified gases is formed beyond the outlet of the cannula to surround the end of the medical instrument. In some configurations, the guiding features are configured to guide the scope within the cannula such that the supplementary gases flow surrounds the endoscope or envelopes the endoscope, and a protection envelope of heated humidified gases is formed beyond the outlet of the cannula to surround the end of the endoscope. The envelope can create a controlled microenvironment around the medical instrument to prevent fogging and deflecting smoke or particles from contacting a lens of the scope, or otherwise obstructing the view of the medical instrument. The envelope can create a controlled microenvironment around the endoscope to prevent fogging and deflecting smoke or particles from contacting a lens of the endoscope.

In some configurations, the guiding features are further configured to orient the medical instrument to prevent flow non-uniformity being formed beyond the outlet of the second cannula. In some configurations, the guiding features are further configured to orient the medical instrument to prevent stagnation zones being formed beyond the outlet of the second cannula. The guiding features may include a plurality of ribs or projections that prevent the medical instrument from resting against a wall of the cannula lumen adjacent the outlet of the cannula. The guiding features may include a plurality of ribs or projections that prevent the endoscope from resting against a wall of the cannula lumen adjacent the outlet of the cannula.

In some configurations, the second cannula may comprise one or more heating elements disposed in the cannula. The heating elements can be configured to heat the gases being delivered into the cavity through the second cannula in order to reduce or prevent condensation.

In some configurations, the surgical system may include a first gas source that is fluidly coupled to the insufflator and a second gas source that is fluidly coupled to the supplementary gas system. The surgical system may be an insufflation system. The first gas source can supply gas to the surgical system for delivery into the body cavity, and the second gas source can supply gas to the supplementary gas system for delivery into the body cavity as supplementary gases. The first gas source and second gas source may be any one of or a combination of a gas bottle, a wall gas source, a pendant system, a blower, and a pump.

In another aspect, the present disclosure relates to a surgical system for providing gases into a body cavity. The surgical system may be an insufflation system. The surgical system includes a surgical system having an insufflator, a first cannula and a conduit, and a supplementary gases system. The insufflator is configured for delivering gases to the first cannula via the conduit. The supplementary gases system is configured to provide a supplementary gases flow into the body cavity in order to maintain a substantially constant pressure within the body cavity.

In some configurations, the supplementary gases system includes a supplementary gas module, a humidifier, a second conduit, and a second cannula, the humidifier being downstream of the supplementary gas module. The humidifier can be configured to heat and humidify gases received from the supplementary gas module and deliver the heated humidified gases to the body cavity through the second cannula and second conduit. The second cannula can be downstream of the humidifier and the second conduit can fluidly couple the second cannula to the humidifier. The supplementary gas module can have one or more control elements configured to control the gas flow such that a substantially continuous gas flow is delivered to the humidifier and the body cavity.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure. In some cases, a “slice” has been shown for clarity purposes for some sectional and cross-sectional views of a three dimensional cannula. A person reasonably skilled in the art would be able to appreciate that these figures illustrate a slice of a three dimensional cannula. In some cases, the projection surfaces have not been shown for clarity. For example, projecting hole surfaces have not been shown in some views.

FIG. 1 illustrates schematically an example medical gases delivery apparatus.

FIGS. 2A-2C illustrate schematically examples of a medical gases delivery apparatus.

FIGS. 3A-3Q illustrate schematically various examples of surgical systems configured to provide continuous gas flow driven by a pressurized gas source to a surgical cannula inserted into a body cavity.

FIGS. 4A-4E illustrate schematically various examples of arrangements of cannulas, including surgical cannulas, insufflation cannulas, and/or pressure release cannulas, that may be used in the surgical systems described herein.

FIGS. 5A-5F illustrate schematically various examples of humidification chambers that comprise elongated pathways for increasing residence time of gas flow within the chamber and, optionally, for acting as a pressure regulator to induce a pressure drop. FIGS. 5A-5C illustrate various views of a spiraling pathway. FIGS. 5D-5F illustrate various views of a raster-patterned pathway.

FIGS. 6A-6D illustrate additional examples of pressure and flow regulators that may be used to modify the pressure and/or flow of gas within a gas flow line of the surgical systems described herein.

FIGS. 7A-7I illustrate schematically various examples of surgical systems comprising a gas recirculation line configured to provide continuous gas flow to a surgical cannula inserted into a body cavity.

FIG. 8 illustrates schematically an example of a combined recirculation and surgical cannula comprising a suction unit configured to recirculate gas from the body cavity through the cannula and over an endoscope and/or other surgical tools.

FIGS. 9A-9C illustrate schematically various examples of suction units that may drive gas flow through a recirculation line.

FIGS. 10A-10G illustrate schematically various examples of surgical systems comprising accumulators configured to store gas from a non-continuous flow of an insufflation line and release the stored gas to provide continuous gas flow to a surgical cannula inserted into a body cavity.

FIGS. 11A-11B illustrate schematically example of non-flexible accumulators. FIG. 11A illustrates a piston-based accumulator. FIG. 11B illustrates a spring-based accumulator.

FIGS. 12A-12B illustrate schematically examples of surgical cannulas incorporating an accumulator in-line with the insufflation flow pathway.

FIGS. 13A-13C illustrate schematically examples of surgical cannulas incorporating an accumulator in-line with a recirculation pathway.

FIGS. 14A-14B illustrate schematically examples of surgical systems comprising feedback lines which can be configured to induce an insufflator to provide continuous gas flow.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

### Example Medical Gases Delivery Systems

Gases can be introduced to a surgical cavity, such as the peritoneal cavity for example via a cannula inserted through an incision made in patient's body (such as the abdominal wall for example). The cannula can be coupled to an insufflator. The gases flow from the insufflator can be increased to inflate the surgical cavity (such as to maintain a pneumoperitoneum, which is a cavity filled with gas within the abdomen, for example). The introduced gases can inflate the surgical cavity. A medical instrument can be inserted through the cannula into the inflated surgical cavity. The medical instrument may be a surgical instrument. The medical instrument may be an endoscope. For example, an endoscope, another scope, camera unit, or other vision system can be inserted into the cavity and visibility in the cavity can be assisted by insertion of fluids, including gases or liquids, such as air or carbon dioxide. The camera unit can include a lens inserted through a trocar. The trocar may include a cannula and obturator. The lens can be connected to a camera positioned, for example, outside the surgical cavity. After initial insufflation and insertion of the instrument (such as a laparoscope for example) through the primary cannula, additional cannulas can be placed in the surgical cavity under laparoscopic observation. Gases and/or surgical smoke can be vented from the surgical cavity using venting features integrated into the cannula, or a venting attachment on one of the cannulas placed in the surgical cavity. At the end of the operating procedure, all instruments and cannulas are removed from the surgical cavity, the gases are expelled, and each incision is closed. For thoracoscopy, colonoscopy, sigmoidoscopy, gastroscopy, bronchoscopy, and/or others, the same or substantially similar procedure for introducing gases to a surgical cavity can be followed. The quantity and flow of gases can be controlled by the clinician performing the examination and/or automatically by the surgical system. The surgical system may be an insufflation system.

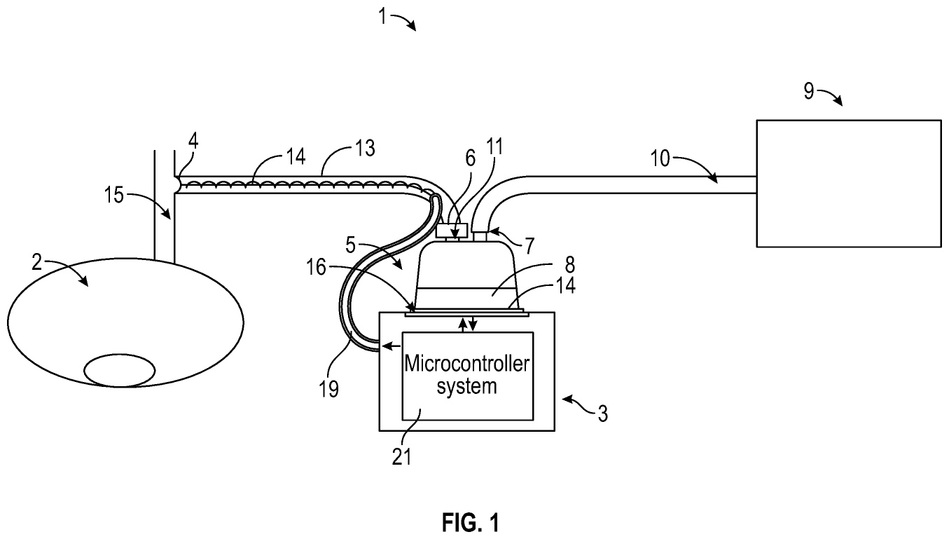

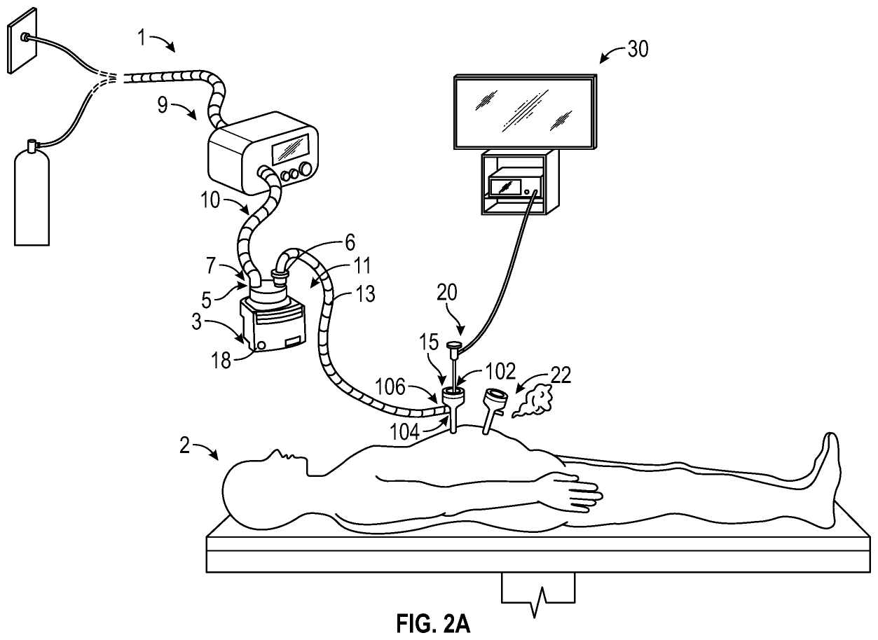

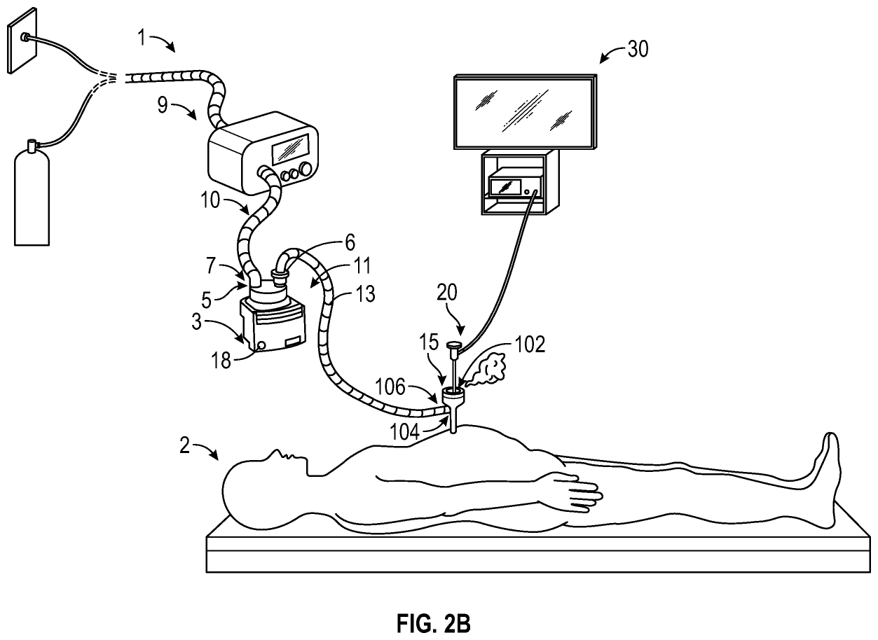

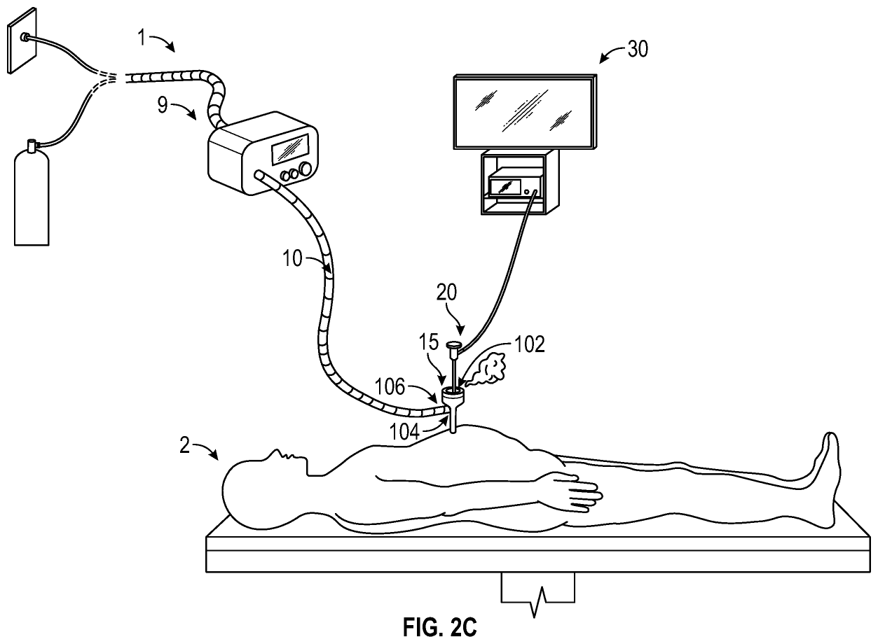

FIGS. 1 and 2A-C illustrate schematically using an example surgical system 1 during a medical procedure. The surgical system may be an insufflation system. Features of FIGS. 1 and 2A-C can be incorporated into each other. The same features have the same reference numerals in FIGS. 1 and 2A-C. As shown in FIG. 1, the patient 2 can have a cannula 15 inserted within a cavity of the patient 2 (for example, an abdomen of the patient 2 in the case of a laparoscopic surgery), as previously described.

As shown in FIGS. 1 and 2A-C, the cannula 15 can be connected to a gases delivery conduit 13 (for example, via a Luer lock connector 4). The cannula 15 can be used to deliver gases into a surgical site, such as within the cavity of the patient 2 for example. The cannula 15 can include one or more passages to introduce gases and/or one or more medical instruments 20 into the surgical cavity. The medical instruments may be surgical instruments. The medical instrument can be a scope, electrocautery tool, or any other instrument. The medical instrument 20 can be coupled to an imaging device 30, which can have a screen. The imaging device 30 can be part of a surgical system, which can include a plurality of surgical tools and/or apparatuses. The surgical system could be a surgical stack. In some configurations, the cannula 15 can be used in a system that includes a supplementary gases source.

As shown in FIG. 2A, the system can include a venting cannula 22, which can have substantially the same features as the cannula 15. The venting cannula may include a leak device coupled to the venting cannula. The leak device may include a valve that allows and/or controls venting. The valve can be automatically controlled by a controller associated with the gases source (i.e. insufflator) or by a controller in the humidifier. A controller may be associated with both the gases source (e.g., insufflator) and the humidifier. The controller associated with the gases source and/or the humidifier may be external from the gases source and/or the humidifier. The controller may also be positioned internally within the cannula. The valve can also be manually actuated (for example, by turning a tap by hand or by a foot pedal, or otherwise). The leak device can include a filtration system to filter out smoke and the like. The venting cannula 22 can also alternatively be coupled to a recirculation system (see FIG. 23) that is configured to recirculate the gases from the surgical cavity back to the insufflator for re-delivery into the surgical cavity. The gases can also be filtered and/or dehumidified prior to being returned to the insufflator. As shown in FIGS. 2B and 2C, the cannula 15 can include a venting attachment so that a venting cannula 22 may not be necessary. The cannula 15 may include two or more passages. One passage can be configured to deliver gases and/or the medical instrument into the surgical cavity. Another passage can be configured to vent gases out of the surgical cavity.

As shown in FIGS. 1, 2A and 2B, the gases delivery conduit 13 can be made of a flexible plastic and can be connected to a humidifier chamber 5. The humidifier chamber 5 can optionally or preferably be in serial connection to a gases supply 9 via a further conduit 10. The gases supply or gases source can be an insufflator (e.g., a blower incorporated into the insufflator), bottled gases, or a wall gases source. The gases supply 9 can provide the gases without humidification and/or heating. A filter 6 be connected downstream of the humidifier's outlet 11. The filter can also be located along the further conduit 10, or at an inlet of the cannula 15. The filter can be configured to filter out pathogens and particulate matter in order to reduce infection or contamination of the surgical site from the humidifier or gases source. The gases supply can provide a continuous or intermittent flow of gases. The further conduit 10 can also preferably be made of flexible plastic tubing.

As shown in FIGS. 2A and 2B, the humidifier chamber 5 may be a separate device from an insufflator gases supply 309. The humidifier chamber 5 may be coupled to an insufflator gases supply 309 by a conduit 10. Alternatively or additionally, a humidifier may be incorporated into the insufflator gases supply 309 (not shown). For instance, the insufflator gases supply 309 and humidifier chamber 5 may share a housing. In some embodiments, whether separated or combined, the insufflator gases supply 309 and humidifier chamber 5 may share a single controller. For instance, the insufflator controller may be in wired or wireless communication with the humidifier heater base unit 3, described elsewhere herein. Alternatively, a humidifier controller may be used to adjust the functions of the insufflator gases supply 9 via wired or wireless communication. In some embodiments, whether separated or combined, the insufflator gases supply 309 and humidifier chamber 5 may comprise separate and operably independent controllers, as described elsewhere herein.

The gases supply 9 can provide one or more insufflation gases, such as carbon dioxide for example, to the humidifier chamber 5. The gases can be humidified as they are passed through the humidifier chamber 5, which can contain a volume of humidification fluid 8, such as water for example. The gases can also be humidified in a humidifier chamber 5 that is heated or non-heated. The gases can be dry cold gas, dry hot gas, humidified gas, or otherwise. Optionally, the gases supply 9 can include two gas sources.

A humidifier that incorporates the humidifier chamber 5 can be any type of humidifier. The humidifier chamber 5 can include a plastic formed chamber having a metal or otherwise conductive base 14 sealed thereto. The base can be in contact with the heater plate 16 during use. The volume of humidification fluid 8 contained in the chamber 5 can be heated by a heater plate 16, which can be under the control of a controller or control means 21 of the humidifier. The humidification fluid 8 may be water. The volume of humidification fluid 8 within the chamber 5 can be heated such that it evaporates, mixing humidification fluid vapor (e.g., water vapor) with the gases flowing through the chamber 5 to heat and humidify the gases.

The controller or control means 21 can be housed in a humidifier base unit 3, which can also house the heater plate 16. The heater plate 16 can have an electric heating element therein or in thermal contact therewith. One or more insulation layers can be located between in the heater plate 16 and the heater element. The heater element can be a base element (or a former) with a wire wound around the base element. The wire can be, for example, a nichrome wire (or a nickel-chrome wire). The heater element can also include a multi-layer substrate with heating tracks electrodeposited thereon or etched therein. The controller or control means 21 can include electronic circuitry, which can include a microprocessor for controlling the supply of energy to the heating element. The humidifier base unit 3 and/or the heater plate 16 can be removably engageable with the humidifier chamber 5. The humidifier chamber 5 can also alternatively or additionally include an integral heater. Alternatively, the controller or control means 21 can be housed or partially housed external to the humidifier base unit 3.

The heater plate 16 can include a temperature sensor, such as a temperature transducer for example or otherwise, which can be in electrical connection with the controller 21. The heater plate temperature sensor can be located within the humidifier base unit 3. The controller 21 can monitor the temperature of the heater plate 16, which can approximate a temperature of the humidification fluid 8.

A temperature sensor can also be located at the or near the outlet 11 to monitor a temperature of the humidified gases leaving the humidifier chamber 5 from the outlet 11. The temperature sensor can also be connected to the controller 21 (for example, with a cable or wirelessly). Additional sensors can also optionally be incorporated, for example, for sensing characteristics of the gases (such as temperature, humidity, flow, or others, for example) at a patient end of the gases delivery conduit 13.

The gases can exit out through the humidifier's outlet 11 and into the gases delivery conduit 13. The gases can move through the gases delivery conduit 13 into the surgical cavity of the patient 2 via the cannula 15, thereby inflating and maintaining the pressure within the cavity. Preferably, the gases leaving the outlet 11 of the humidifier chamber 5 can have a relative humidity of up to 100%, for example the relative humidity can be 100%. As the gases travel along the gases delivery conduit 13, further condensation can occur so that humidification fluid can condense on a wall of the gases delivery conduit 13. Further condensation can have undesirable effects, such as detrimentally reducing the fluid content of the gases delivered to the patient for example. In order to reduce and/or minimize the occurrence of condensation within the gases delivery conduit 13, a heating element, such as, for example, a heater wire 14 can be provided within, throughout, or around the gases delivery conduit 13. The heater wire 14 can be electronically connected to the humidifier base unit 3, for example by an electrical cable 19 to power the heater wire. In some embodiments, other heating elements could be included in addition or alternatively, e.g., a conductive ink, or a flexible PCB. In some cases, the PCB could be flexible, or rigid and pre-shaped to an arcuate shape for example. In some embodiments, the heating element could be, for example, discrete Positive Temperature Coefficient (“PTC”) heaters, or heaters including conductive plastic/polymer. Optionally, the heating element can include an inductive heating element. Optionally, the heating element can include a chemical heating element, for example, silica beads. Optionally, the cannula can be pre-heated prior to insertion.

The heater wire 14 can include an insulated copper alloy resistance wire, other types of resistance wire, or other heater element, and/or be made of any other appropriate material. The heater wire can be a straight wire or a helically wound element. An electrical circuit including the heater wire 14 can be located within walls of the gases delivery tube 13. The gases delivery tube 13 can be a spiral wound tube. Alternatively, the gases delivery tube 13 can include a non-helical or straight tube. Optionally, the gases delivery tube 13 can be corrugated or non-corrugated. The heater wire 14 can be spirally wound around an insulating core of the gases delivery conduit 13. The insulating coating around the heater wire 14 can include a thermoplastics material which, when heated to a predetermined temperature, can enter a state in which its shape can be altered and the new shape can be substantially elastically retained upon cooling. The heater wire 14 can be wound in a single or double helix. Measurements by the temperature sensor and/or the additional sensor(s) at the patient end of the conduit 13 can provide feedback to the controller 21 so that the controller 21 can optionally energize the heater wire to increases and/or maintain the temperature of the gases within the gases delivery conduit 13 (for example, between approximately 35° C. and 45° C.) so that the gases delivered to the patient at the desired temperature, which can be at or close to 37° C. or above or below the internal body temperature (for example, approximately 1° C., 2° C., 3° C., 4° C., 5° C., 6° C., 7° C., 8° C., 9° C., 10° C., or 15° C. above or below 37° C.).

The controller or control means 21 can, for example, include the microprocessor or logic circuit with associated memory or storage means, which can hold a software program. When executed by the control means 21, the software can control the operation of the surgical system 1 (such as an insufflation system, for example) in accordance with instructions set in the software and/or in response to external inputs. For example, the controller or control means 21 can be provided with input from the heater plate 16 so that the controller or control means 21 can be provided with information on the temperature and/or power usage of the heater plate 16. The controller or control means 21 can be provided with inputs of temperature of the gases flow. For example, the temperature sensor can provide input to indicate the temperature of the humidified gases flow as the gases leave the outlet 11 of the humidifier chamber 5. A flow sensor can also be provided in the same position as or near the temperature sensor or at other appropriate location within the surgical system 1. The controller 21 can control a flow regulator which regulates the flow rate of gases through the system 1. The regulator can include a flow inducer and/or inhibiter such as a motorized fan or pump for example. Valves and/or vents can additionally or alternatively be used to control the gases flow rate.

A patient input 18 located on the humidifier base unit 3 can allow a user (such as a surgeon or nurse for example) to set a desired gases temperature and/or gases humidity level to be delivered. Other functions can also optionally be controlled by the user input 18, such as control of the heating delivered by the heater wire 14 for example. The controller 21 can control the system 1, and in particular to control the flow rate, temperature, and/or humidity of gas delivered to the patient, to be appropriate for the type of medical procedure for which the system 1 is being used.

The humidifier base unit 3 can also include a display for displaying to the user the characteristics of the gas flow being delivered to the patient 2.

Although not shown, the humidifier can also optionally be a passover or bypass humidifier, which can include the chamber with a volume of water or any other type of humidification fluid, but may not include a heater plate for heating the humidification fluid (for example, water). The chamber can be in fluid communication with the insufflation fluid(s) (for example, gases) supply such that the insufflation fluid(s) are humidified by the humidification fluid vapor (for example, water vapor) evaporated from the volume of humidification fluid (for example, water) as the insufflation gases pass over the volume of humidification fluid.

When in use, the humidifiers described above can be located outside an “operating sterile zone” and/or adjacent the insufflator. As a result, the medical personnel would not be required to touch the humidifier when moving the cannula during the operation to maneuver the medical instruments within the surgical cavity. The humidifier may not need to be sterilized to the same extent as the medical instruments. Furthermore, the humidifier being located outside the “operating sterile zone” can reduce obstructions to the medical personnel during the operating procedure that may restrict movements of the medical personnel and/or the medical instruments in the already crowded space.

As shown in FIG. 2C, the system may be used without a humidifier so that the gases supply 9 can be coupled directly to the cannula 15.

### Examples of Continuous Flow Systems

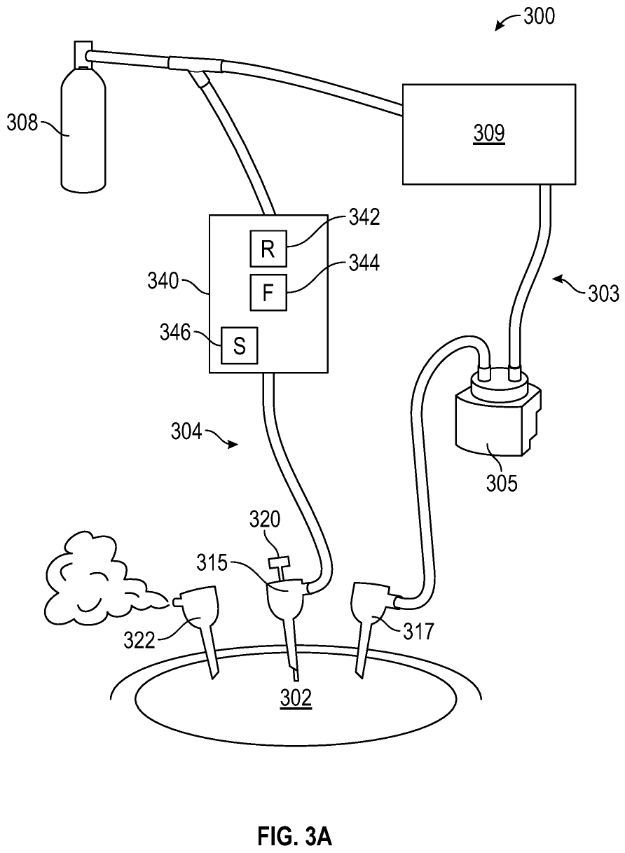

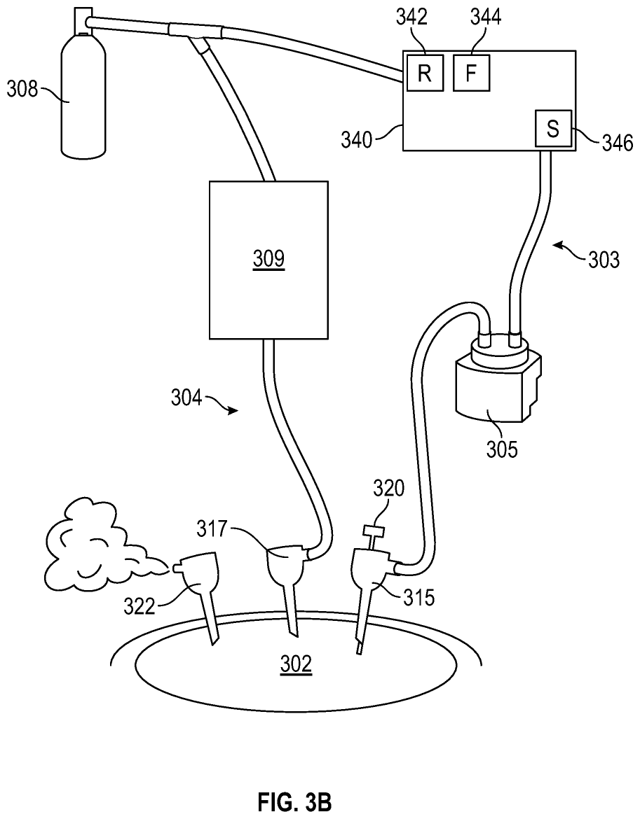

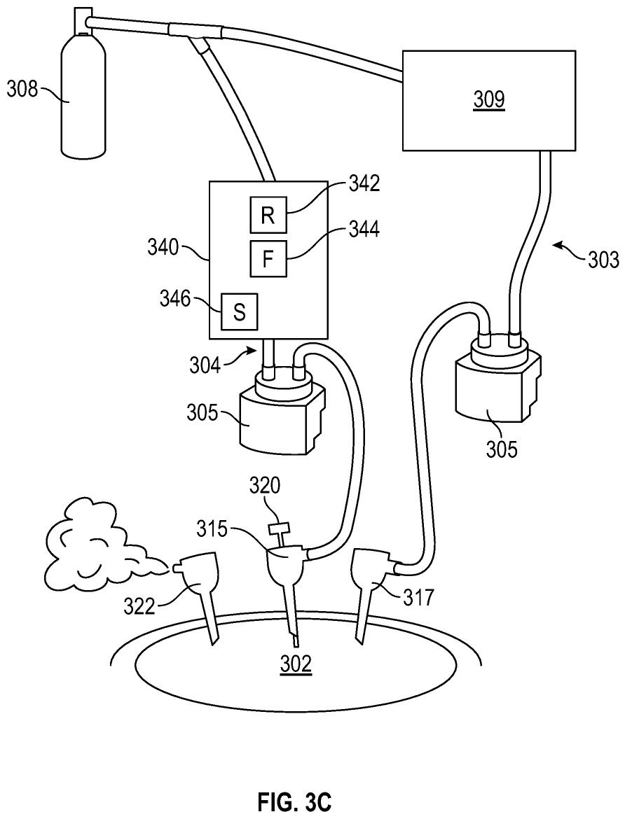

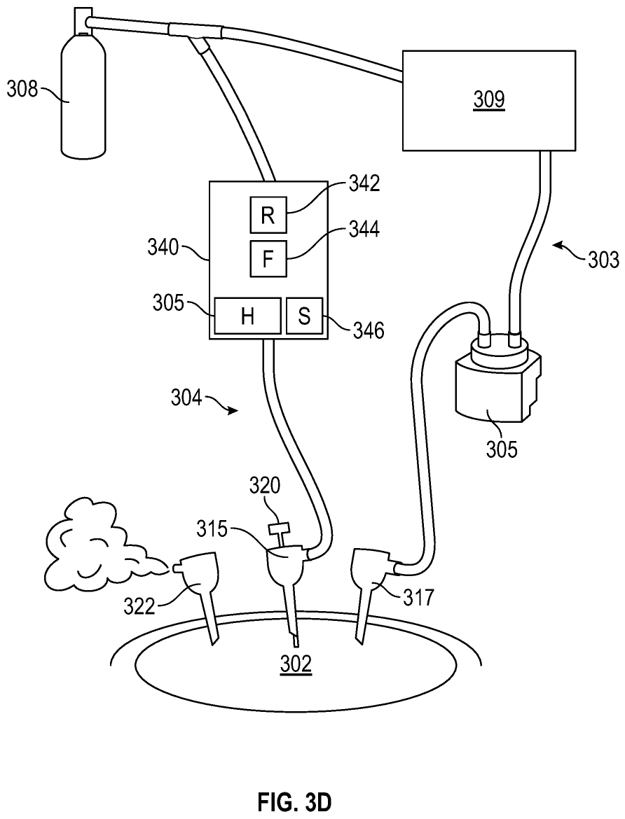

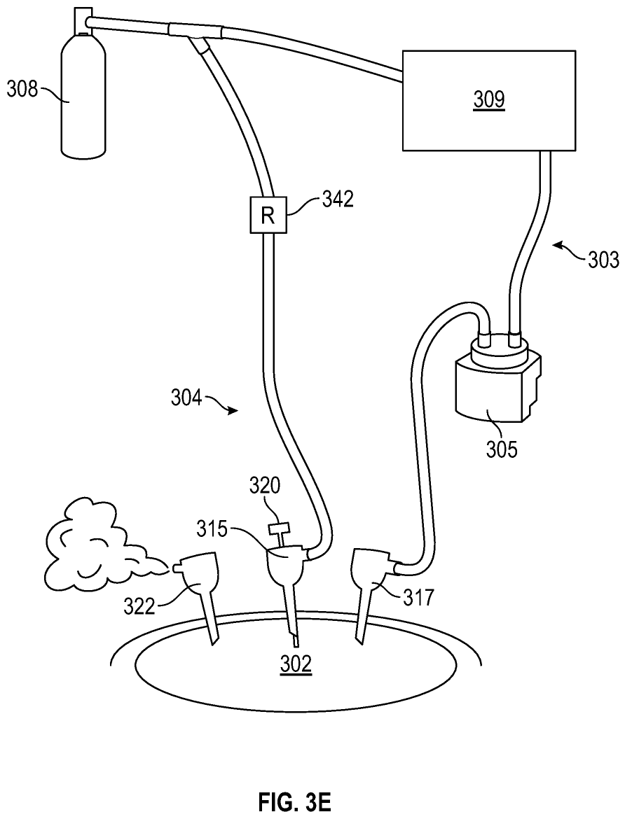

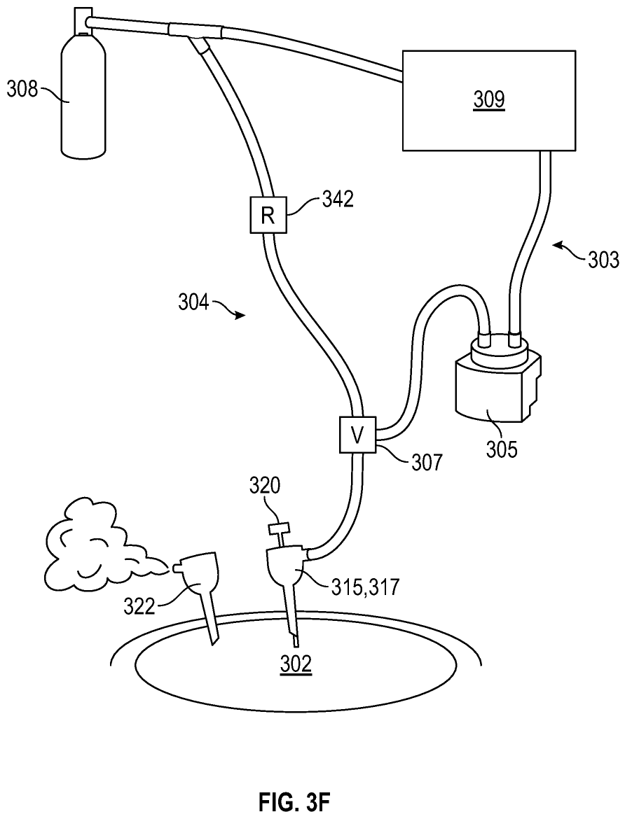

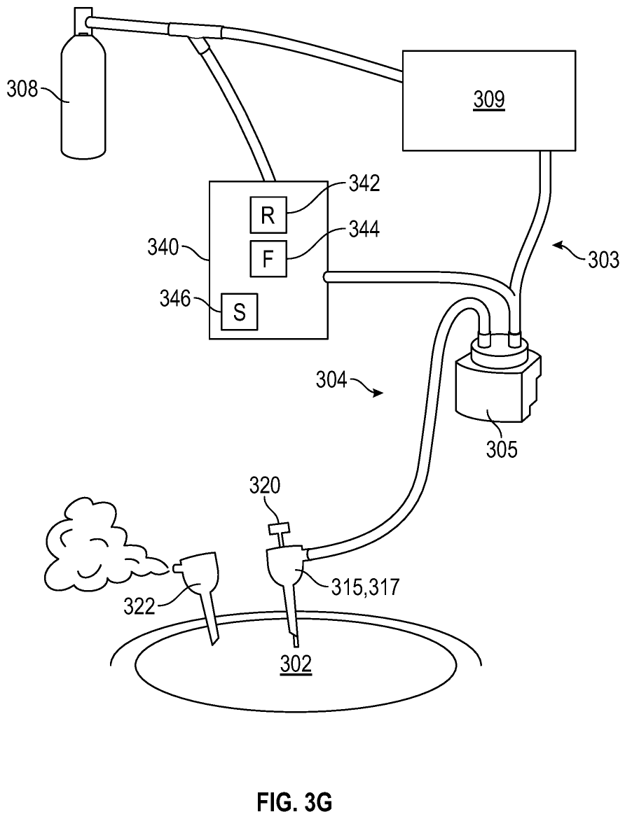

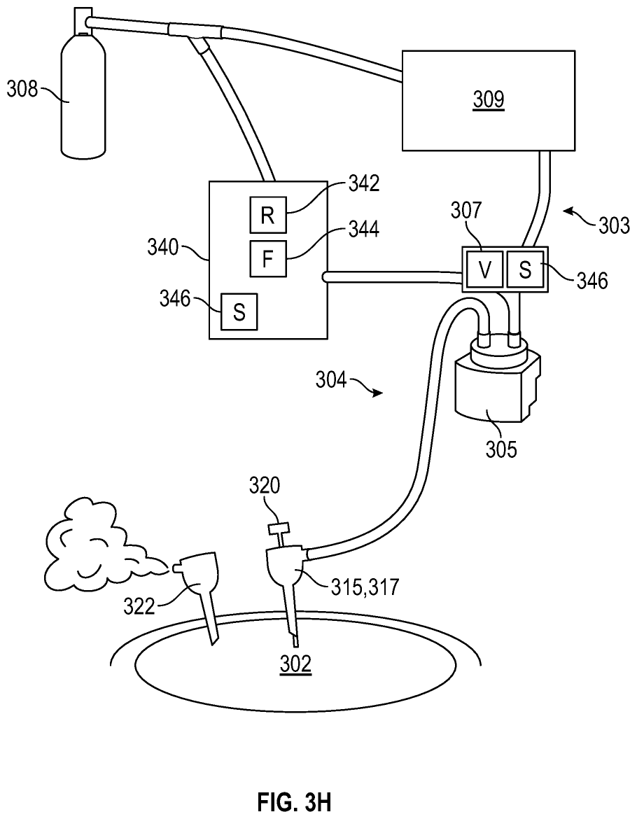

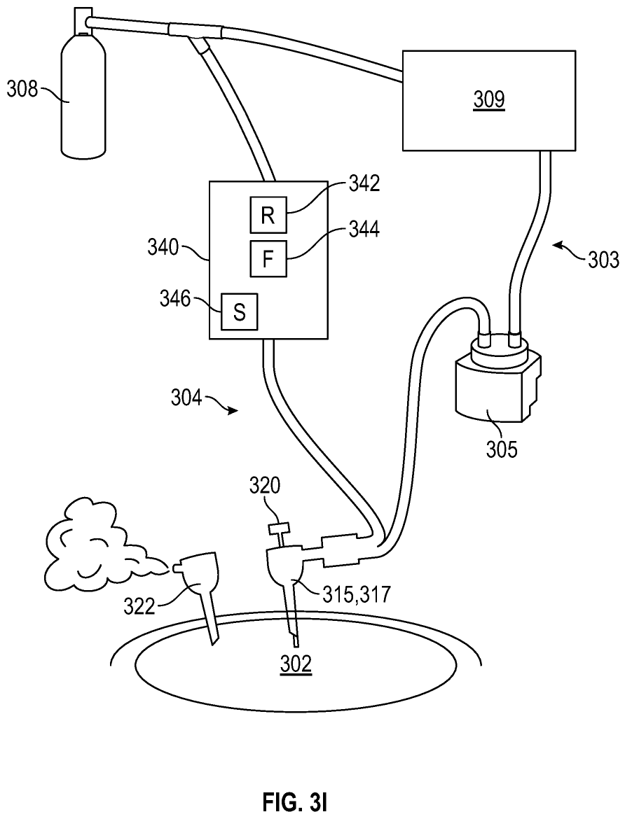

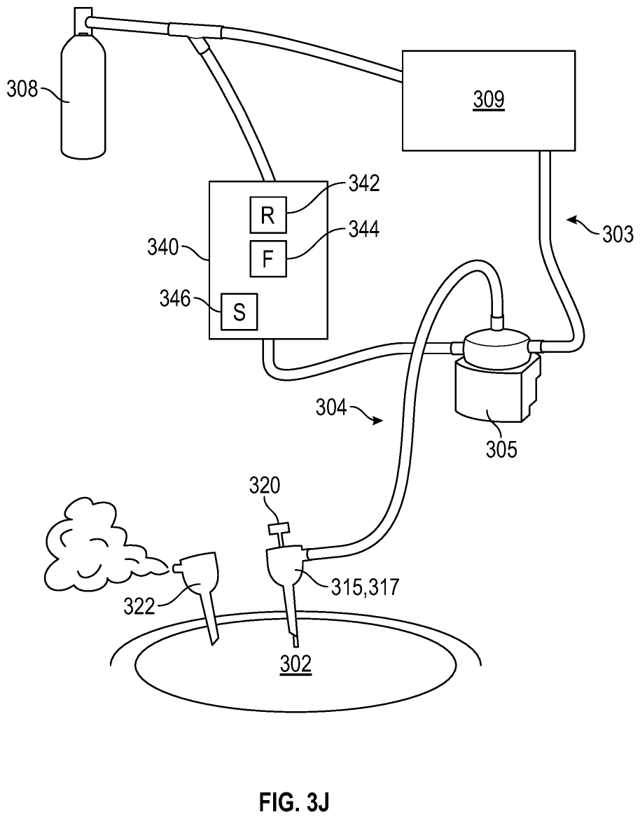

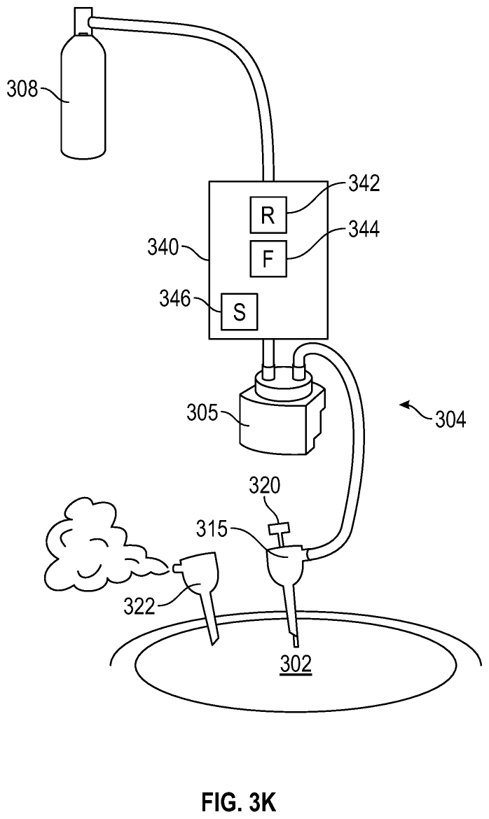

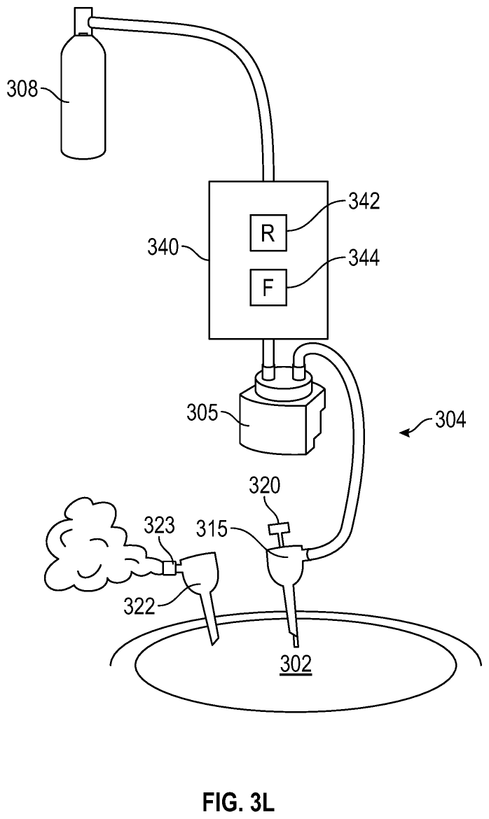

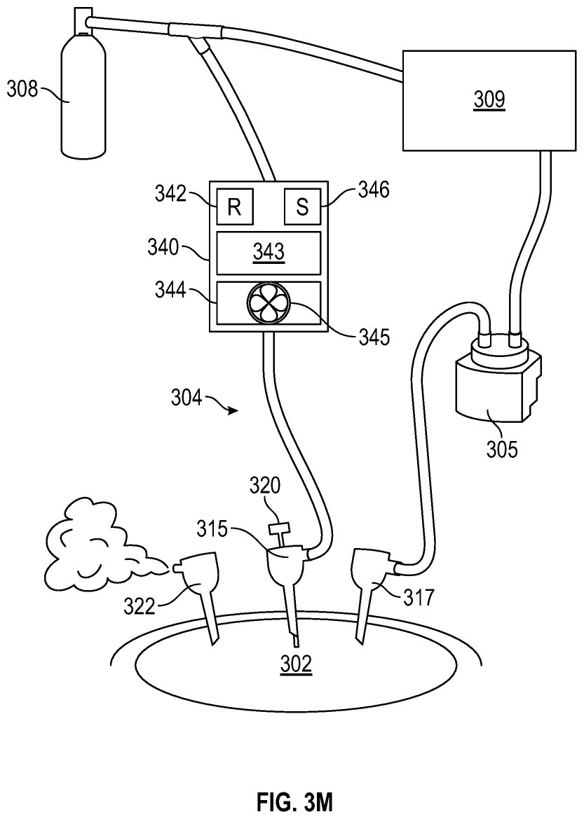

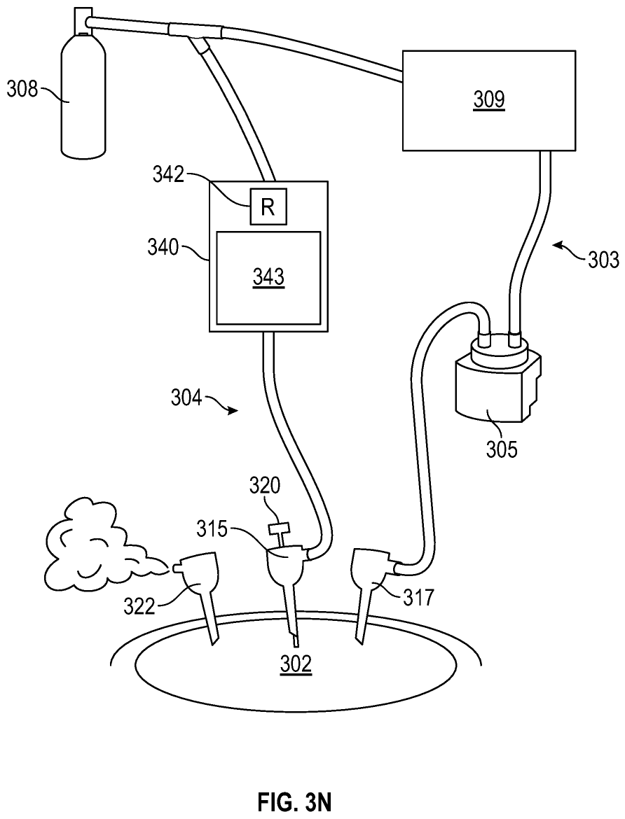

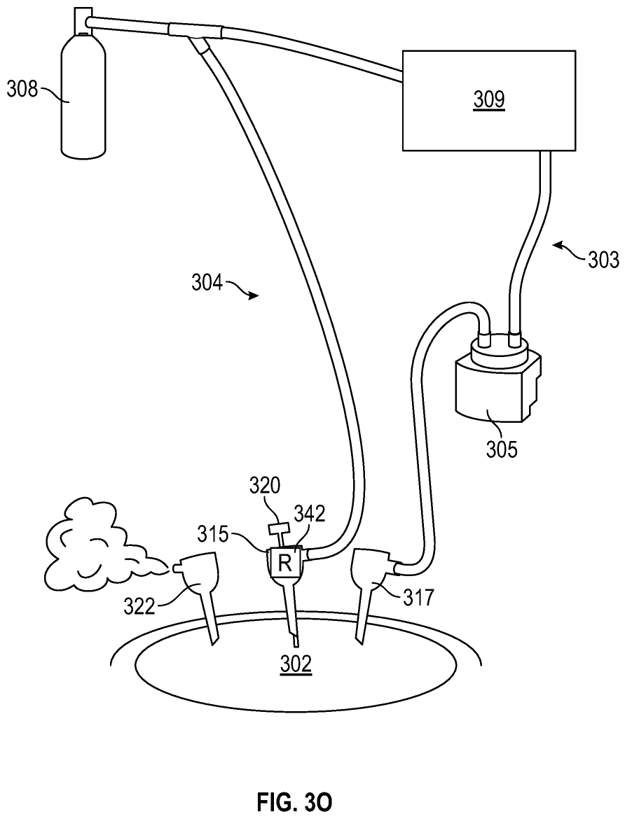

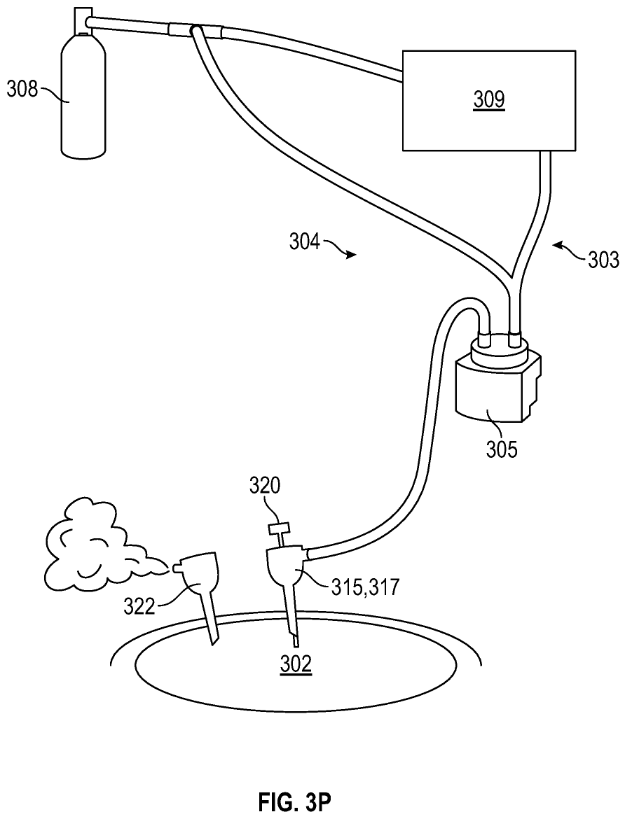

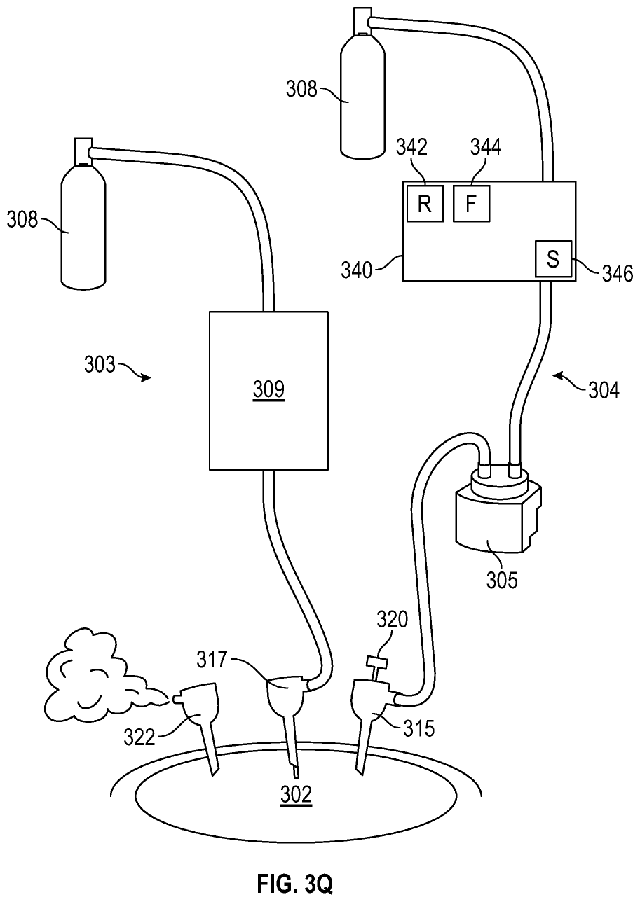

FIGS. 3A-3Q schematically illustrate various examples of a surgical system 300 configured to provide a continuous flow of gas to a cannula 315. The surgical system could be an insufflation system. Some of the examples illustrated may comprise the same functional components assembled in different arrangements. Some of the examples may comprise additional components relative to other examples. Some of the illustrated components may be optional. In some implementations, various components of the surgical system 300 may comprise standard components from a surgical system that is not specifically designed to provide a continuous gas flow to the cannula 315, but which are assembled with additional components and/or modified to achieve continuous gas flow as described. In some implementations, all of the components may be specifically configured to achieve continuous gas flow as described. As one non-limiting example, one or more humidifiers may be on either or both of a continuous flow line or insufflation flow line within the system.

FIG. 3A depicts a surgical system 300 comprising two independently regulated lines, an insufflation line 303 and a continuous flow line 304, in fluid communication with a downstream body cavity 302, such as a pneumoperitoneum for example. Any surgical systems referred to herein may be, for example, insufflation systems. The insufflation line 303 and the continuous flow line 304 may share a common upstream gas source 308 (e.g., carbon dioxide) or different gas sources (not shown). The gas source 308 may provide a source of pressurized gas (e.g., a gas tank or wall source) which flows through the insufflation line 303 and/or continuous flow line 304 down a pressure gradient. In embodiments in which the lines 303, 304 share a single gas source 308, the lines may share a common conduit (e.g., tubing) in fluid communication with the gas source 308 at the proximal ends of the lines 303, 304, which may split into separate conduits (e.g., via a Y-connector).

The surgical system 300 may comprise one, two, three, or more cannulas in fluid communication with the body cavity 302 (e.g., inserted through surgical incisions in the peritoneum). For example, the surgical system 300 may comprise a surgical cannula 315 for receiving one or more medical instruments such as an endoscope 320 for example, an insufflation cannula 317 for introducing the insufflation gas into the body cavity 302, and/or a venting device 322 (e.g., a cannula) configured to vent flow of gases from the body cavity 302. Any medical instruments referred to herein may be, for example, surgical instruments. The surgical cannula 315 may be coupled to or may form a distal end of the continuous flow line 304 such that the continuous flow line 304 provides a continuous gas flow around at least a portion of a medical instrument received in the surgical cannula 315, such as around a lens of an endoscope 320 for example. The insufflation cannula 317 may be coupled to or may form a distal end of the insufflation line 303 such that the insufflation gas is supplied to the body cavity 302 through the insufflation cannula 317. The venting device 322 may not be coupled to any lines. The venting device 322 may be in fluid communication with the ambient environment outside the body cavity 302. The venting device 322 may release pressure and/or vent gas flow from the body cavity 302 to maintain a desired (e.g., constant) pressure and/or to vent smoke from electrosurgical procedures. For instance, the venting device 322 may help maintain pressure within the body cavity 302 below a threshold pressure (e.g., 50 mmHg). Pressure in the body cavity 302 may be controlled by an insufflator which incorporates pressure relief features. The venting device 322 may comprise a one-way valve that only allows fluid (e.g., gas) transport from the body cavity 302 to the ambient environment and not vice-versa. In some embodiments, the venting device 322 may comprise and/or may be coupled to one, two, or more filters for filtering the released gas before it is allowed to escape into the ambient atmosphere. The venting device 322 may be configured to constantly release pressure (e.g. leak gas). In some embodiments, the venting device 322 may vent flow at a constant flow rate. In some embodiments, the venting device 322 may have an adjustable and/or openable/closeable release valve as described elsewhere herein. In some embodiments, the venting device 322 may have a valve configured to open and release pressure above a threshold pressure. In some embodiments, the venting device 322 may be a standard cannula with a tap. In some embodiments, the venting device 322 may be a device that connects to the standard cannula. The venting device 322 may comprise a flow restriction that is dimensioned and shaped to allow a controller venting flow. The venting flow may be more than or equal to the flow of gases delivered to the cavity, so that it can be ensured that the patient is not over-pressurized. In some embodiments, the venting device 322 may comprise a tap or an extraction device that is configured to vent gases at a predetermined flow rate.

The cannula or venting attachment can also include one, two, or more filters configured to remove potentially hazardous chemicals and/or particles before releasing the gases and/or smoke into the atmosphere. The filter can be configured to filter particles as small as about 0.1 microns to about 0.2 microns, or about 0.12 microns. The filter can be configured to filter the particles with at least about 99% efficiency, or about 99.999% efficiency, or about 99.9995% efficiency. The filter can be an ultra-low particulate air (ULPA) filter. The filter can also include optionally a carbon filter to reduce odor. The filters could also be a high-efficiency particulate air (HEPA) filter. The filters can include multiple filter elements that can be positioned in series. For example, ULPA filters and carbon filters can be positioned in series, for example.

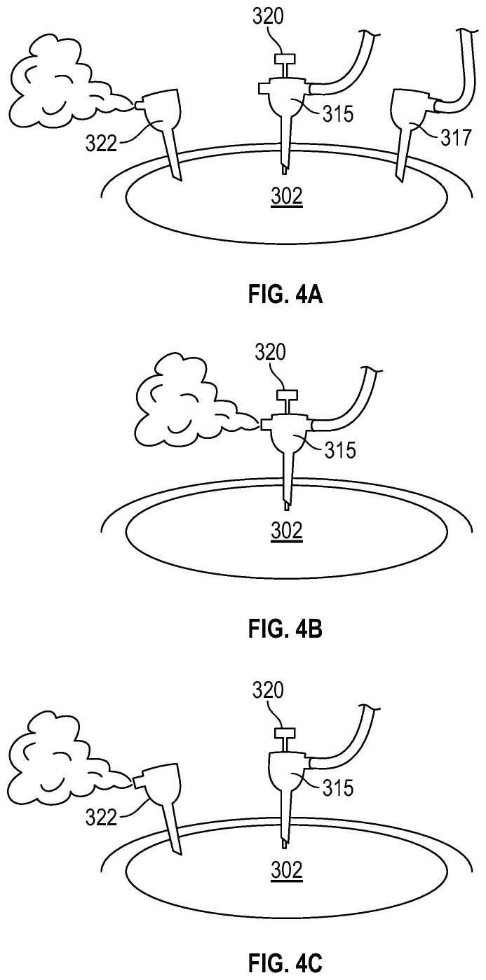

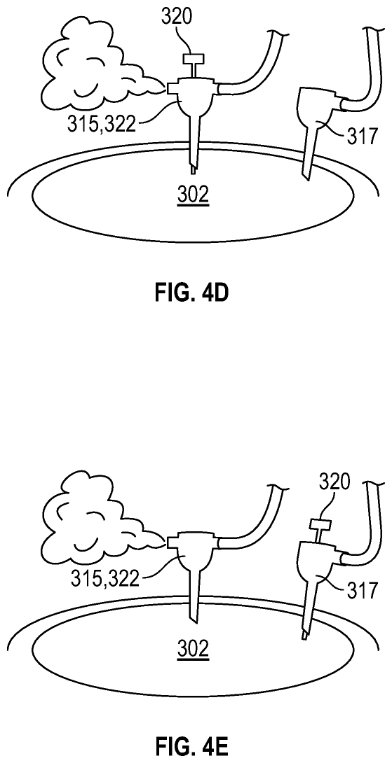

In some embodiments, a single cannula may serve two or more of the functions of the surgical cannula 315, insufflation cannula 317, and/or the venting device 322, as described elsewhere herein (e.g., FIGS. 4A-4E). Any one or more of the cannulas described herein may be modified to communicate (e.g., via cables or wirelessly) with power sources and/or data communications line for providing advanced electronically-actuated functionality, such as modulating pressure regulation or controlling a suction device for example, as described elsewhere herein. Any of the conduits disclosed herein as part of a gas flow line may be adapted or modified to also transfer power and/or data. In some embodiments, one or more of the cannulas, such as the surgical cannula 315 for example, may be heated to help prevent or at least reduce condensation on the endoscope 320 and/or other medical instruments. Additionally or alternatively, the temperature of the gas flow through the cannulas can be regulated.

The continuous flow line 304 may comprise a supplementary gases module or unit 340 operatively positioned between the gas source 308 and the surgical cannula 315. The supplementary gases module 340 may comprise a pressure regulator 342, such as any pressure regulator commonly known in the art for example, for reducing the pressure of the gas supplied by the gas source 308 to a predetermined pressure level while maintaining a flow from the gas source 308 to match the downstream demand flow. In various embodiments, the pressure regulators 342 may comprise relief valves for releasing pressure over a threshold pressure, which may be set or designated by the pressure regulator 342. The supplementary gases module 340 may comprise a flow control 344, such as any flow control commonly known in the art for example, for modulating the flow rate of gas through the downstream portion (downstream of the supplementary gases module 340) of the continuous flow line 304. The flow control 344 can include, for instance, a flow inducer and/or inhibiter such as a motorized fan for example. Valves and/or vents can additionally or alternatively be used to control the flow rate. The supplementary gases module 340 may comprise one or more sensors 346 such as for sensing the pressure, flow rate, temperature, and/or humidity in the downstream portion of the continuous flow line 304 for example. The sensors 346 may be positioned downstream of the pressure regulator 342 and/or the flow control 344. The sensed pressure may be equal or substantially equal to the pressure within the downstream body cavity 302 or at least indicative of the downstream pressure through a formulaic relationship. In some embodiments, the pressure regulator 342 and/or the flow control 344 may be adjustable (e.g., automatically) in response to measurements from the one or more sensors 346. For instance, the flow control 344 may automatically respond to a sensed increase in pressure by increasing a resistance to gas flow so as to maintain a constant volumetric flow rate within the downstream portion of the continuous flow line 304.

The supplementary gases module 340 may be an integrated unit (e.g., housed within a single console) or may comprise one or more various components interconnected through gas flow conduits. The supplementary gases module 340 may comprise an electronic controller, including electronic circuitry, which may share features with controller 21 for instance. The supplementary gases module 340, for instance, can include a microprocessor or logic circuit with associated memory or storage means, which can hold a software program. When executed by the supplementary gases module 340, the software can control the operation of the continuous flow line 304 (or the entire surgical system 300) in accordance with instructions set in the software and/or in response to external inputs. For example, the supplementary gases module 340 can be provided with feedback from the one or more sensors 346. The supplementary gases module 340 may comprise input features (not shown) configured to allow a user (such as a surgeon or nurse for example) to set one or more desired parameters, such as pressure, flow rate, temperature, and/or humidity downstream of the module 340 for example.

The insufflation line 303 may comprise an insufflation module or insufflator 309 operatively positioned between the gas source 308 and the insufflation cannula 317 for regulating gas flow through the insufflation line 303. The insufflator 309 may be any insufflator 309 commonly known in the art. In some embodiments, the insufflator 309 may comprise one or more of the same or similar components as the supplementary gases module 340. The insufflator 309 may comprise, for example, a pressure regulator, flow control, and/or one or more various sensors. The insufflator 309 may cycle between phases of positive flow rate and no flow, producing a pulsatile flow. The insufflator 309 may, for example, provide positive flow when the pressure level within the body cavity 302 drops below a threshold pressure (e.g., as measured by a pressure sensor in the insufflation line 303) to maintain a minimum pressure and/or volume within the body cavity 302. The insufflator 309 may be an integrated unit (e.g., housed within a single console) or may comprise one or more various components interconnected through gas flow conduits. The insufflator 309 may be configured as an electric controller similar to the supplementary gases module 340 and/or controller 21. In some embodiments, one or more controllers may electronically operate various combinations of the insufflation line 303 (including the humidifier 305) and the continuous flow line 304, such that any one or more of the controllers described as separate units herein may be operatively and/or physically combined into single units.

As shown in FIG. 3A, a humidifier 305, such as a Humigard™ System (Fisher & Paykel Healthcare, Auckland, NZ) for example, may be operatively positioned within the insufflation line 303 in series with the insufflator 309. The humidifier 305 may comprise the same or similar features as the humidifier chamber 5 disclosed elsewhere herein. The humidifier 305 may be positioned downstream of the insufflator 309, as shown in FIG. 3A, or it may be positioned upstream of the insufflator 309. FIGS. 3B-3D illustrate examples of alternative positioning of the humidifier 305. In some configurations, humidifiers 305 may be positioned within the insufflation line 303 and/or the continuous flow line 304. In some embodiments, the humidifier 305 may be positioned within the continuous fluid flow line 304 (e.g., downstream of the continuous flow console 340), as shown in FIG. 3B. In some embodiments, humidifiers 305 may be positioned both within the insufflation line 303 and the continuous flow line 304, as shown in FIG. 3C. In some embodiments, the humidifier 305 and the insufflator 309 may be housed or packaged in a single unit or console. For example, a humidifier 305 may be incorporated into the continuous flow console 340, as shown in FIG. 3D.

In some embodiments, the continuous flow line 304 may not comprise a flow control 344 and/or sensors 346. The pressure in the downstream portion of the continuous flow line 304 may be set by a pressure regulator 342 as shown in FIG. 3E. The flow rate through the downstream portion of the continuous flow line 304 may be driven entirely by the pressure differences. Gas flow may be driven by a pressure difference between the downstream portion of the continuous flow line 304 and the ambient environment. The continuous flow line 304 and the ambient environment may be in constant fluid communication through the venting device 322 such that gas flow is constantly escaping (e.g., leaking) through the venting device 322 and being replenished by the high pressure gas source 308 via the continuous flow line 304. In some implementations, gas flow may be at least partially driven by transient pressure differences between the body cavity 302 and the pressure set by a pressure regulator 342. The humidifier 305 may be positioned downstream of the insufflator 309, as shown in FIG. 3F, or it may be positioned upstream of the insufflator 309. In some embodiments, humidifiers 305 may be positioned both within the insufflation line 303 and/or the continuous flow line 304.

In some embodiments, the insufflation line 303 and the continuous flow line 304 may merge or converge into a single line along a downstream portion. FIGS. 3F-3J depict examples converging insufflation and continuous flow lines 303, 304. In some configurations, humidifiers 305 may be positioned in either or both of the converging insufflation and continuous flow lines 303, 304. The distal ends of the lines 303, 304 may end at a single surgical cannula 315 which also serves as the insufflation cannula 317. The lines 303, 304 may converge downstream of the insufflator 309 and the continuous flow pressure regulator 342 (e.g., alone or as part of the supplementary gases module 340). The lines 303, 304 may converge downstream of humidifiers 305 in the insufflation line 303 and/or continuous flow line 304, as depicted in FIG. 3F, and/or upstream of a humidifier 305, as depicted in FIG. 3G. FIG. 3G depicts an insufflation line 303 and continuous flow line 304 which converge at the inlet of a humidification chamber of a humidifier 305. In some implementations, conduits from each of the lines 303, 304 may be coupled to the inlet of the humidifier 305 via a Y-shaped connector. In some implementations, the humidifier 305 may comprise two inlets into the humidification chamber, one for coupling to the insufflation line 303 and one for coupling to the continuous flow line 304. The supplementary gases module 340 within the continuous flow line 304 may be configured to provide a supplementary or auxiliary flow of gases to the body cavity 302 (e.g., the pneumoperitoneum) which compensates for the insufflator 309 (e.g., a standard off-the shelf insufflator) shutting off (off-phases) when the insufflator 309 detects an appropriate pressure above a pressure threshold within the body cavity 302. The supplementary gases module 340 facilitates providing a continuous flow of gases into the surgical cannula 315. The flow pattern of gas flow out of the supplementary gases module 340 may be steady (constant flow rate), pulsatile, or irregular.

In some embodiments, the insufflation line 303 and continuous flow line 304 may be interconnected by a fluid flow valve 307 where the lines merge. In some embodiments, the valve 307 may be configured to switch between gas flow from the insufflation line 303 and the continuous flow line 304. The valve 307 may be configured to switch to the continuous flow line 304 whenever gas is not being provided through the insufflation line 303. For instance, gas may only be passed through the valve 307 from the continuous flow line 304 during off phases of a pulsatile gas flow provided through the insufflation line 303. The flow rate of gas through the continuous flow line 304 may be less than the flow rate of gas during positive phases or pulses of flow through the insufflation line 303. FIG. 3F depicts a valve 307 operatively positioned downstream of the insufflator 309 and a humidifier 305 positioned in the insufflation line 303 and downstream of a pressure regulator 342 in the continuous flow line 304. FIG. 3H depicts a valve 307 operatively positioned downstream of the insufflator 309 in the insufflation line 303 and downstream of a pressure regulator 342 in the continuous flow line 304, but upstream of a shared humidifier 305. In some embodiments, the surgical system 300 may comprise one or more sensors 346, such as a pressure sensor for example, positioned downstream of the valve 309. The one or more sensors 346 may be positioned between the valve 309 and the humidifier 305. The one or more sensors 346 may be configured to control the flow of gas to the humidifier 305 by switching the valve 309. In some embodiments, the insufflation line 303 and the continuous flow line 304 may converge at a shared humidifier 305 as shown in FIG. 3J. The humidifier 305 may comprise separate input ports for accepting or coupling to separate conduits from the insufflation line 303 and the continuous flow line 304. The humidifier 305 may comprise a shared output port for a shared conduit comprising the downstream portion of the combined insufflation line 303 and continuous flow line 304.

In some embodiments where the insufflation line 303 and the continuous flow line 304 converge, they may share one or more downstream conduits extending, for example, to the surgical cannula 315, as shown in FIGS. 3F, 3G, 3H, and 3J. In some embodiments, where the insufflation line 303 and the continuous flow line 304 converge, they may comprise independent conduits but be coupled to the same surgical cannula 315, as shown in FIG. 3I. The surgical cannula 315 may be modified to accept or be coupled to two or more gas lines. For instance, the surgical cannula 315 may comprise two fluid ports for attaching to conduits (e.g., tubing) from various gas flow lines.

In some embodiments, the surgical system 300 may not comprise an insufflation line 303 and/or an insufflator 309 at all. For example, as shown in FIGS. 3K and 3L, the only gas line communicating gas into the body cavity 302 may be the continuous flow line 304. In some configurations, humidifiers 305 may be positioned in the continuous flow line 304. The continuous flow line 304 may comprise a supplementary gases module 340 and/or a humidifier 305 as described elsewhere herein. The supply of a continuous flow of gas through the surgical cannula 315 over the endoscope 320 and/or other surgical tool or tools may at least partially expand the volume of and/or increase the pressure inside of the body cavity 302. Accordingly, the continuous flow line 304 may entirely replace or supplant the function of the insufflation line 303. The accumulating pressure of the gas introduced into the body cavity 302 via the continuous flow line 304 may be relieved through a venting device 322 as described elsewhere herein. In some embodiments, the venting device 322 may release (e.g., leak) gas from the body cavity 302 at a constant flow rate or at a non-adjustable flow rate proportional to the pressure gradient between the body cavity 302 and the ambient environment, as schematically depicted in FIG. 3K. One or more sensors, such as a pressure sensor 346 for example, which may be part of the continuous flow control module 340 may be used to provide feedback and adjust the flow rate of the continuous flow line 304 so that the body cavity 302 does not become over-pressurized and/or under-pressurized. In some embodiments, as shown in FIG. 3L, the venting device 322 may comprise an adjustable valve 323 or other mechanism which may modulate the release of gas from the body cavity 302 through the cannula 322. The adjustable valve 323 may be manually operable by a user and/or automatically operated by the surgical system (e.g., in response to pressure readings). For instance, the user may turn or rotate the pressure release valve 323 in one direction (e.g., clockwise) to increase the flow rate of pressure released from the body cavity 302 and turn or rotate the pressure release valve 323 an opposite direction (e.g., counterclockwise) to decrease the flow rate of pressure released from the body cavity 302.

In some embodiments, the pressure release valve 323 may be an integral part of the venting device 322. In some embodiments, the pressure release valve 323, may be an attachable component that is removably attachable to a standard (e.g., non-adjustable) venting device 322 (e.g., coupled to an output port of the cannula 322). In some embodiments comprising an adjustable pressure release valve 323, the continuous flow line 304 may not comprise sensors 346 for regulating the gas flow (e.g., pressure sensors), as shown in FIG. 3L, and/or the continuous flow of gas may be provided at a constant or non-adjustable flow rate. The pressure and/or volume within the body cavity 302 may be entirely regulated via the adjustable pressure release valve 323.

In some embodiments, the supplementary gases module 340 may comprise a low pressure gas storage 343 (the pressure being lower than the upstream pressure of the gas source 309). FIGS. 3M and 3N illustrate embodiments of the surgical system 300 comprising low pressure gas storages 343 in the continuous flow line 304. In some configurations, humidifiers 305 may be positioned within the insufflation line 303 and/or the continuous flow line 304. A pressure regulator 342 as described elsewhere herein may be operatively positioned between the gas source 309 and the low pressure gas storage 343. The pressure regulator 342 may set the pressure of the gas within the low pressure gas storage 342.

In some embodiments, the flow control 344 may comprise a fan element 345 configured to drive gas flow from the low pressure gas storage 343 downstream to the body cavity 302 through the surgical cannula 315, as shown in FIG. 3M. The fan element 345 may drive positive flow downstream at a rate above which the natural pressure gradient between the low pressure gas storage 343 and the body cavity 302 would drive flow (e.g., even if the pressure in the low pressure gas storage 343 is equal to the pressure in the body cavity 302). In some embodiments, a sensor 346 may be a pressure sensor configured to measure pressure within the low pressure gas storage 343. The pressure sensor may regulate the fan element 345. For instance, when the pressure in the low pressure gas storage exceeds a threshold pressure, the fan element 345 may be actuated to drive gas flow. In some embodiments, the fan element 345 may always be running. In other embodiments, the fan element 345 may switch between off and on phases. Continuous gas flow may be driven by pressure gradients during phases when the fan element 345 is off. In some embodiments, the fan element 345 may run at a fixed speed. In some embodiments, the fan element 345 may operate at a variable speed which depends on the sensed pressure within the low pressure gas storage 343. The fan element 345 may decrease speed (and accordingly flow rate) as the pressure drops, such as when the low pressure gas storage 343 is refilling for example. The fan element 345 may increase speed as the pressure increases.

In some embodiments, as depicted in FIG. 3N, the low pressure gas storage 343 may be configured to be positioned vertically above the body cavity 302 such that there is a height differential between the low pressure gas storage 343 and the body cavity 302. A conduit between the low pressure gas storage 343 and the body cavity 302 may be configured to extend substantially in a downward direction between the low pressure gas storage 343 and the body cavity 302 such that the gas flowed through the conduit is not required to travel in a substantially uphill direction. The volume of gas stored within the low pressure gas storage 343 may create a head pressure under the force of gravity which may be used to drive gas flow downstream to the body cavity 302 through the surgical cannula 315. The head pressure may drive positive flow downstream at a rate above which the natural pressure gradient between the low pressure gas storage 343 and the body cavity 302 would drive flow (e.g., even if the pressure in the low pressure gas storage 343 is equal to the pressure in the body cavity 302).

In some embodiments, gas flow through the continuous flow line 304 may be driven entirely by pressure gradients regulated by a pressure regulator 342 as described with respect to FIG. 3E. In some embodiments, the pressure regulator 342 may not be a separate or discreet component installed within the continuous flow line 304 but may be incorporated into another component of the surgical system 300 through which the continuous flow line 304 extends. In some configurations, humidifiers 305 may be positioned within the insufflation line 303 and/or the continuous flow line 304. For example, FIG. 3O depicts an example of a surgical system 300 in which the pressure regulator 342 is incorporated directly into the surgical cannula 315. The pressure regulator 342 may be an integral part of the surgical cannula 315 or it may be removably attached to or coupled to the surgical cannula 315 (e.g., attached to an input port of the surgical cannula 315). FIG. 3P depicts an example of a surgical system 300 in which the pressure regulator 342 is incorporated directly into a humidifier 305. The pressure regulator 342 may be an integral part of the humidifier 305 or it may be removably attached to or coupled to the humidifier (e.g., attached to an input port of the surgical cannula 315). As shown in FIG. 3P, the pressure regulator 342 may be installed in a downstream portion of the insufflation line 303 at point after which the insufflation line 303 and the continuous flow line 304 converge. In some configurations, humidifiers 305 may be positioned within the insufflation line 303 and/or the continuous flow line 304.

FIG. 3Q depicts an example of a surgical system 300 in which the insufflation line 303 and the continuous flow line 304 are coupled to separate and independent pressurized gas sources 308. In some configurations, humidifiers 305 may be positioned within the insufflation line 303 and/or the continuous flow line 304. The gas sources 308 may be any suitable gas source, including those described elsewhere herein (e.g., blowers, bottles or tanks, wall sources, etc.). The gas sources 308 may be the same type as one another or different types. Providing separate gas sources 308 may allow for a continuous flow line 304 that is entirely separate and independent from the insufflation line 303 as shown in FIG. 3Q or at least separate and independent upstream of the insufflator 309. Accordingly, the continuous flow line 304 can be operable entirely independent of an insufflator 309 such as a standard off-the shelf insufflator for example. The gases flow rate through the continuous flow line 304 can be independently controlled via the separate gas source 308 and the supplementary gases module 340. The supplementary flow rate can supplement or “top up” the flow from the insufflator 309, such as when the insufflator 309 reduces flow and/or switches off for example. The pressure regulator 342 of the supplementary gases module 340 acts as a safety device which restricts the pressure delivered to the body cavity 302 through the continuous flow line 304. The supplementary gases module 340 may be configured to vent gases flow to the ambient environment (e.g., atmosphere) if the pressure within the continuous flow line 304 or the body cavity 302 exceeds a threshold pressure. The continuous flow line 304 may be configured to provide an independent flow of gas into the body cavity 302, which may be humidified via a humidifier 305 and/or heated (e.g., via surgical cannula 315) as described elsewhere herein. The surgical cannula 315 may comprise an endoscope lumen having a guiding element, as described elsewhere herein, which maintains the endoscope 320 in a concentric position within the endoscope lumen and/or prevents the endoscope 320 from resting against a wall of the endoscope lumen.

FIGS. 4A-4E schematically illustrate examples of various combinations of cannulas that may be used to implement the surgical systems 300 described herein, including embodiments described in FIGS. 3A-3Q. As described elsewhere herein, various embodiments of the surgical systems 300 may comprise three distinct cannulas, as depicted in FIG. 4A: a surgical cannula 315, an insufflation cannula 317, and a venting device 322. The functions of any two or three of the cannulas can be combined into a single cannula as depicted in FIGS. 4B-4E. FIG. 4B illustrates a single surgical cannula 315 which is configured to provide both continuous and non-continuous (e.g., pulsatile) gas flow via converged insufflation and continuous flow lines 303, 304 as well as provide pressure release (e.g., a constant leak) through the surgical cannula 315. The pressure release may occur through a separate internal lumen in fluid communication with the body cavity 302 other than that which receives the endoscope 320 and through which the pressurized gas flow is passed through in order not to interrupt the flow of gas over the endoscope 320. The entrance to the pressure release lumen may be positioned on an outer surface (e.g., an outer diameter) of the surgical cannula 315, such as on a shaft portion for example, which may be positioned concentrically outside of an inner lumen which receives the endoscope 320 and through which gas flow from the insufflation line 3 and the continuous flow line 304 travels into the body cavity 302. In some embodiments, the insufflation line 303 and the continuous flow line 304 converge to a common surgical cannula 315 and a separate venting device 322 is provided, as depicted in FIG. 4C. In some embodiments, a separate or additional venting device 322 may be provided in addition to a surgical cannula 315 and/or an insufflation cannula 317 which may include integrated pressure release mechanisms. In some embodiments, the surgical system 300 may comprise a distinct surgical cannula 315 and a distinct insufflation cannula 317, but pressure release may be incorporated into either the surgical cannula 315, as shown in FIG. 4D, or the insufflation cannula 317, as shown in FIG. 4E.

In various embodiments, the continuous flow line 304 and/or the insufflation line 303 may comprise one or more pressure regulators 342. The pressure regulators 342 described herein may be any standard or common pressure regulator known in the art. In any of the described embodiments, one or more of the pressure regulators 342 may be a non-standard pressure regulator as described herein.

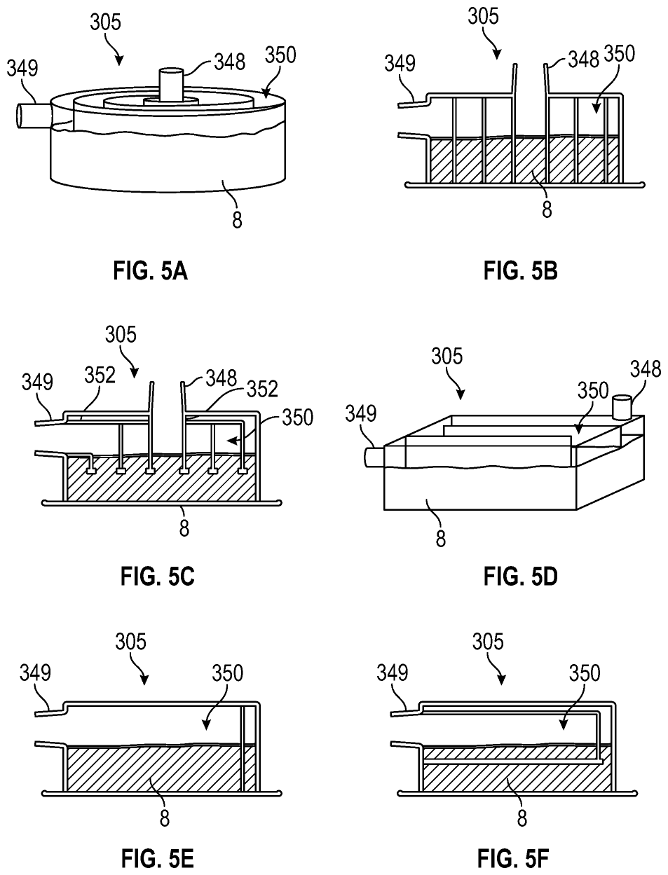

FIGS. 5A-5F schematically depict two examples of a humidifier 305 which use geometrically complex gas pathways 350 to prolong the duration of time the gas flow resides within the chamber of the humidifier 308. The humidifiers 305 may comprise relatively elongated gas flow pathways 350 (e.g., relative to the residual length of the insufflation line 303 or the continuous flow line 304 gas flow pathway) having relatively small cross-sectional areas (e.g., relative to the average cross-sectional area of the insufflation line 303 or the continuous flow line 304 gas flow pathway). The elongated flow pathways 350 may improve humidification by increasing residence time due to various internal walls of the chamber. The internal walls may extend the gas flow pathway within the humidifier 305 chambers. In some implementations, the elongated gas flow pathways 350 may simultaneously serve to establish a predetermined pressure drop between a fluid input 348 and a fluid output 349 such that the elongated gas flow pathway 350 acts as a pressure regulator 342. The pathways 350 may be disposed to substantially fill a 2-dimensional surface area (or 3-dimensional volume) have a surface area substantially greater than the cross-sectional area of the pathway 350. The enhanced friction arising from the elongated length of the gas flow pathway 350 and/or the reduced cross-sectional area of the gas flow pathway 350 may result in a relatively non-negligible pressure drop between the input 348 and output 349. The magnitude of the pressure drop may depend on the flow rate, the pressure of the upstream gas source 308, the pressure of the downstream body cavity 302, and/or the pressure of the ambient environment positioned downstream via the venting device 322. Without being limited by theory, the pressure drop may be generally described by the Darcy-Weisbach equation, depicted in Eq. 1, which relates pressure loss due to friction along a given length of a pipe to the average fluid flow velocity through the pipe, where Δp is the pressure drop, L is the length, f_{D} is the Darcy friction factor, ρ is the fluid density, ν is the mean flow velocity, and D is the hydraulic diameter:

$$Δ p = L \cdot {f}_{D} \cdot \frac{ρ}{2} \cdot \frac{{v}^{2}}{D} \qquad [ Eq . 1 ]$$

FIGS. 5A-5C depict a pressure regulator 342 comprising a spiraling gas flow pathway 350. FIG. 5A depicts a perspective view of the gas flow pathway 350 and FIG. 5B depicts a cross-sectional view of the gas flow pathway 350 shown in FIG. 5A. In some embodiments, the fluid input 348 may be positioned at a center of the spiral (e.g., approaching from a top or bottom of the spiral) and the fluid output 349 may be positioned at a periphery of the spiral (e.g., extending laterally away from the spiral), as shown in FIGS. 5A-5C. In other embodiments, the fluid input 348 and fluid output 349 may be reversed. In various embodiments, the spiral pathway 350 may comprise a plurality of windings (e.g. at least 5, 50, or 500). The humidifier 305 may comprise a chamber 351 which may be equivalent to the humidifier chamber 5 holding a volume of humidification fluid 8, as described elsewhere herein. The volume of humidification fluid 8 may be contained entirely or at least partially within the gas flow pathway 350. In some embodiments, the gas flow pathway 350 may be suspended or floating within the chamber 351, as depicted in FIG. 5C, showing a cross-sectional view of a variant of the regulator 342 shown in FIGS. 5A and 5B. The spiral pathway 350 may be suspended within the chamber 351 via tubing 352 which fluidly connects an input 348 of the pathway 350 with an input port of the chamber 351 and which fluidly connects an output 349 of the pathway 350 with an output port of the chamber 351. As shown in FIG. 5C, the spiral pathway 350 may be suspended near a top portion of the chamber 351. The spiral pathway 350 may comprise an enclosed top surface and an open bottom surface in fluid communication with the volume of humidification fluid 8. The volume of humidification fluid 8 may form a bottom enclosure to the gas flow travelling through the gas flow pathway 350, which may flow over the surface of the volume of humidification fluid 8 due to the lower density of the gas. In other embodiments, the spiral pathway 350 may be integrated into a ceiling of the chamber 351, but may not extend entirely to the floor of the chamber 351.

FIGS. 5D-5F depict a variation of the humidifier 305 illustrated in FIGS. 5A-5C. FIGS. 5D-5F depict a humidifier 305 comprising a continuous raster-patterned or reciprocating linear gas flow pathway 350. FIG. 5D depicts a perspective view of the gas flow pathway 350 and FIG. 5E depicts a cross-sectional view of the gas flow pathway 350 shown in FIG. 5D. The raster-pattern gas flow pathway 350 may define a rectangular or square shaped area. In some embodiments, the fluid input 348 may be positioned at the corner of the pathway 350 (e.g., approaching from a top, bottom, or lateral side of the pathway) and the fluid output 349 may be positioned at an opposite corner of the pathway (e.g., extending from a top, bottom, or lateral side of the pathway), as shown in FIGS. 5D-5F. In various embodiments, the raster-pattern pathway 350 may comprise multiple rows (e.g., at least about 5, 50, or 500 rows) connected into a continuous pathway. In some embodiments, the gas flow pathway 350 may be suspended or floating within the chamber 351, as depicted in FIG. 5F, showing a cross-sectional view of a variant of the regulator 342 shown in FIGS. 5D and 5E. The construction may be the same or similar to that described with respect to FIG. 5C. In other embodiments, the raster pathway 350 may be integrated into a ceiling of the chamber 351, but may not extend entirely to the floor of the chamber 351. Any other suitable configuration of a continuous elongated gas flow pathway 350, such as any 2-D or 3-D maze-like pathway for example, may be used in the humidifier 305 and/or regulator 342.

In various embodiments, the pressure regulator 342 may comprise a pressure release feature which prevents the downstream pressure (e.g., in the continuous flow line 304) from exceeding a predetermined pressure. In some embodiments, the pressure regulator 342 may comprise standard pressure release valves known in the art. In some embodiments, the pressure regulator 342 may comprise non-standard pressure release valves 354 as described herein.

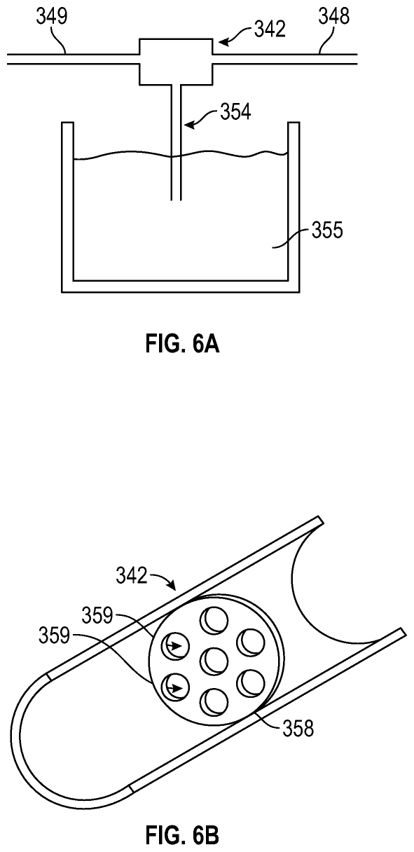

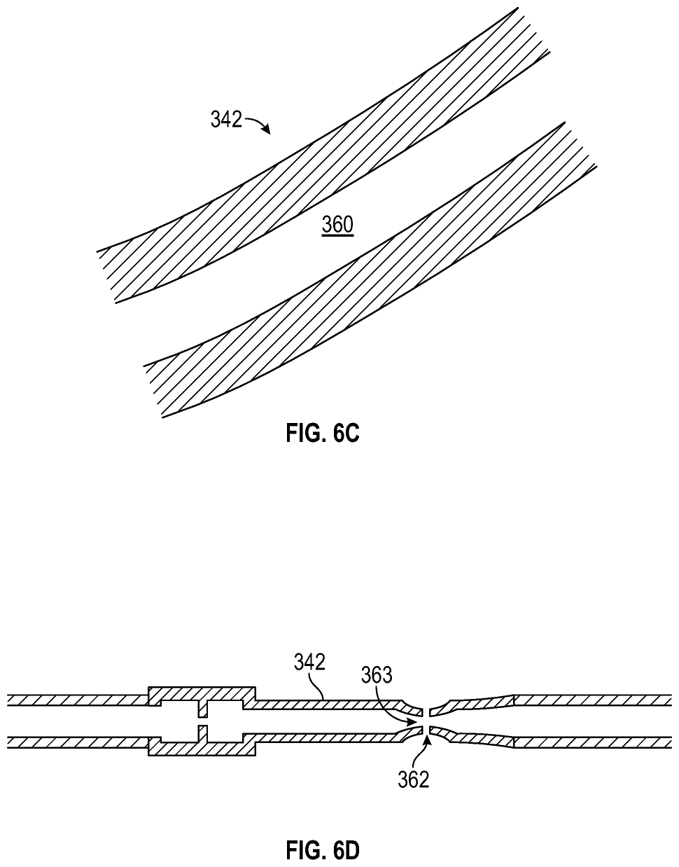

FIG. 6A schematically illustrates a pressure regulator 342 comprising a pressure release valve 354. A line (e.g., the continuous flow line 304) may be positioned vertically above a humidification fluid bath 355 (or a bath of another liquid fluid, such as mineral oil for example). The pressure release valve 354 may comprise a shaft having a lumen that extends vertically downward into the humidification fluid bath 355 placing the gas flow line (e.g., the continuous flow line 304) in fluid communication with the humidification fluid bath 355. The pressure release valve 354 may comprise a generally tubular configuration. The vertical orientation of the gas flow line and the humidification fluid bath 355 may generally prevent humidification fluid (or another other liquid fluid) from entering the gas flow line and/or flowing downstream along the gas flow line since gravity retains the humidification fluid within a lower portion of the pressure release valve 354. The humidification fluid within the humidification fluid bath 355 may rise to a height within the pressure release valve 354 determined by a pressure differential between a pressure of the gas flowing within the gas line and/or regulator 342 and a pressure of the ambient environment over the humidification fluid bath 355. The pressure of the ambient environment may be atmospheric pressure. For instance, a pressure within the gas flow line greater than the pressure of the ambient environment will cause the humidification fluid level within the pressure release valve 354 to be at a lower height than the humidification fluid level of the humidification fluid bath 355 around the pressure release valve 354. If the pressure within the gas flow line exceeds a particular threshold pressure, the humidification fluid level within the pressure release valve 354 may fall low enough that the gas is forced out the bottom of the pressure release valve 354 into the humidification fluid bath 355 where the gas can escape (e.g., bubble) to the ambient environment above the humidification fluid bath 355. Accordingly, pressure may be prevented or inhibited from rising above the threshold pressure in a downstream portion of the gas line as excess pressure will escape through the humidification fluid bath 355. The depth that a shaft of the pressure release valve 354 extends into the humidification fluid bath (below the humidification fluid level of the humidification fluid bath 355) may modulate the threshold pressure at which pressure is relieved from the gas flow line. Shafts that extend deeper into the humidification fluid bath 355 may require a larger drop in the humidification fluid level within the pressure release valve 354 for pressure relief to occur and, accordingly, the threshold pressure will be set higher. In some embodiments, the pressure regulator 342 may be incorporated into a humidifier as shown in FIG. 3P. In some embodiments, the humidification fluid bath 355 may be the volume of humidification fluid 8 stored in the humidifier.

Any of the pressure regulators 342 described herein may comprise a pressure drop feature, such as but not limited to those described with respect to FIGS. 5A-5F for example, and/or a pressure release feature, such as but not limited to that described with respect to FIG. 6A for example. Some embodiments may comprise only a pressure drop feature and some embodiments may comprise only a pressure release feature. Some embodiments of the surgical system 300 may employ more than one pressure regulator 342 in a given gas flow line. For instance, the continuous flow line 304 may comprise a non-standard pressure regulator 342 positioned downstream of a standard pressure regulator 342 or vice-versa. Relief of upstream pressure will result in a pressure drop in the gas flow line across the pressure relief feature. Pressure drops in the gas line will generally result in a reduced gas flow rate downstream of the pressure drop for a fixed downstream pressure.

FIG. 6B schematically illustrates another example of a pressure regulator 342. The pressure regulator 342 may comprise an orifice plate 358 configured to be installed in the gas flow pathway of a gas flow line (e.g., the continuous flow line 304). The orifice plate 358 may comprise one or more orifices 359 having reduced cross-sectional area. As described elsewhere herein, the restricted cross-sectional area for gas flow across the orifice plate 358 may exert additional resistance to gas flow via friction which causes a pressure drop across the orifice plate 358 and slows the flow of gas across the orifice plate 358 reducing the gas flow rate. In some embodiments, the cross-sectional area of each orifice 359 may comprise no more than approximately 50%, 40%, 30%, 20%, 10%, 5%, or 1% of the cross-sectional area of an unrestricted portion of the gas flow line (e.g., a gas flow conduit) by way of non-limiting example. In some embodiments, the cumulative cross-sectional area of a plurality of orifices 359 may amount to no more than approximately 95%, 90%, 80%, 70%, 60%, 50%, 40%, 30%, 20%, 10%, or 5% of the cross-sectional area of an unrestricted portion of the gas flow line (e.g., a gas flow conduit) by way of non-limiting example. In some embodiments, the orifice plate 358 may comprise at least 1, 2, 3, 4, 5, 10, 15, 20, 25, 50, 75, 100, 150, 200, or 500 orifices 359 by way of non-limiting example. For a fixed cumulative cross-sectional area of the one or more orifices 359, increasing the number of orifices 359 may increase the frictional resistance exerted on the gas flowing through the orifice plate 358 and may result in a larger pressure drop across the orifice plate 358. The resistance and friction may increase with the length of the orifices 359 resulting in larger pressure drops and flow reductions for longer orifices 359. In some embodiments, the pressure regulator 342 may comprise one or more orifice plates 358. The orifice plates 358 may comprise the same or similar configurations, such as the cross-sectional size, number, arrangement, and/or lengths of the orifices 359 for example. The orifice plates 358 may be positioned back-to-back, may be spaced, and/or may be positioned throughout various locations in the gas flow line. In some embodiments, the pressure regulator 342 may comprise a tubular configuration (e.g., cylindrical) as shown in FIG. 6B. The orifice plate 358 may be installed in a removable or non-removable fashion within a gas flow lumen of the pressure regulator 342. In some embodiments, the pressure regulator 342 may be configured to be installed in series with conduits (e.g., tubing) of the gas flow line (e.g., continuous flow line 304). In some embodiments, the pressure regulator 342 may be incorporated into other components of a gas flow line at points beside the conduits of the gas flow line as described elsewhere herein (e.g., the humidifier 305 or surgical cannula 315).

FIG. 6C schematically illustrates another example of a pressure regulator 342. The pressure regulator 342 may comprise a single orifice 360 of restricted diameter that extends a length sufficient to cause a pressure drop as described elsewhere herein. The restricted diameter orifice 360 may function similarly to the elongated pathway 350 described elsewhere herein to reduce downstream pressure. The single orifice 360 may form a substantially linear elongated fluid pathway across which the pressure drops and the gas flow is slowed. In some embodiments, the cross-sectional area of the restricted diameter orifice 360 may comprise a cross-sectional area substantially less than the cross-sectional area of an unrestricted portion of the gas flow line (e.g., 50%, 40%, 30%, 20%, 10%, 5%, or 1% of the cross-sectional area of the unrestricted portion of the gas flow line). The unrestricted portion, for example, may be a gas flow conduit. In some embodiments, the pressure regulator 342 may be installed in series with conduits (e.g., tubing) of a gas line (e.g., continuous flow line 304). In some embodiments, the pressure regulator 342 may be fabricated from flexible material such that the pressure regulator 342 may bend and/or conform to the direction of the gas flow line. In some embodiments, the restricted diameter orifice 360 may be a piece of conduit (e.g., tubing) having a restricted diameter over a non-negligible length. In some embodiments, the pressure regulator 342 may be incorporated into other components of a gas flow line at points beside the conduits of the gas flow line as described elsewhere herein (e.g., the humidifier 305 or surgical cannula 315).

FIG. 6D schematically illustrates another example of a pressure regulator 342. The pressure regulator 342 may one or more apertures (not shown) and/or a gap 362 in a section of a gas flow line (e.g., continuous flow line 304) which creates a fluid communication between an internal flow pathway of the gas line and an ambient environment. In some implementations, the ambient may be at atmospheric pressure. The gap 362 and/or the apertures may be dimensioned such that the flowing gas does not escape through the gap 362 into the ambient environment unless the flowing gas exceeds a threshold pressure. The threshold pressure may depend upon the fluid velocity at which the gas flow passes the gap 362. The gap 362 and/or the apertures may be disposed on a narrowed section 363 of the flow path having a narrowed diameter. The narrowed diameter may increase the gas flow velocity and reduce the pressure along the narrowed section 363 of the gas flow pathway according to the Venturi effect or Bernoulli's principle. In some embodiments, the length of the narrowed section 363 may not be long enough and/or the reduced cross-section of the narrowed section 363 may not be small enough to create a reduced pressure on the downstream end of the narrowed section 363 via friction as described elsewhere herein. In other embodiments, the narrowed section 363 may be configured to create a non-negligible pressure drop across the length of the narrowed section 363 such that the downstream pressure is reduced, in a manner similar to the pressure regulator 342 described with respect to FIG. 6C. In such embodiments, the narrowed section 363 comprising gap 362 may act as pressure regulator 342 configured to create a pressure drop and relieve pressure above a threshold pressure. If the pressure of the flowing gas is reduced in the narrowed section 363, the threshold pressure may be adjusted to accommodate for the expected return to an increased pressure downstream of the narrowed section 363. The narrowed section 363 may transiently increase the fluid velocity of the gas flow, which may advantageously prevent or inhibit entrainment of air from the ambient environment. Higher velocities of gas flow across the gap 363 may discourage air from the ambient environment from entering the gas line and joining the fluid flow. The functioning of the pressure regular 342 shown in FIG. 6D may resemble the pressure release mechanism of a standard Bunsen burner. In some embodiments, the pressure regulator 342 may be installed in series with conduits (e.g., tubing) of a gas line (e.g., continuous flow line 304). In some embodiments, the pressure regulator 342 may be incorporated into other components of a gas flow line at points beside the conduits of the gas flow line as described elsewhere herein (e.g., the humidifier 305 or surgical cannula 315).

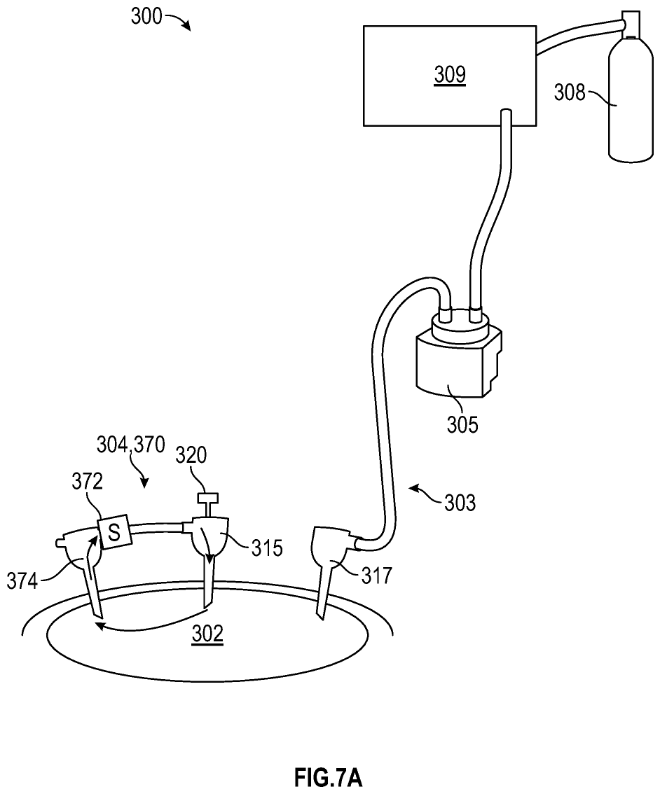

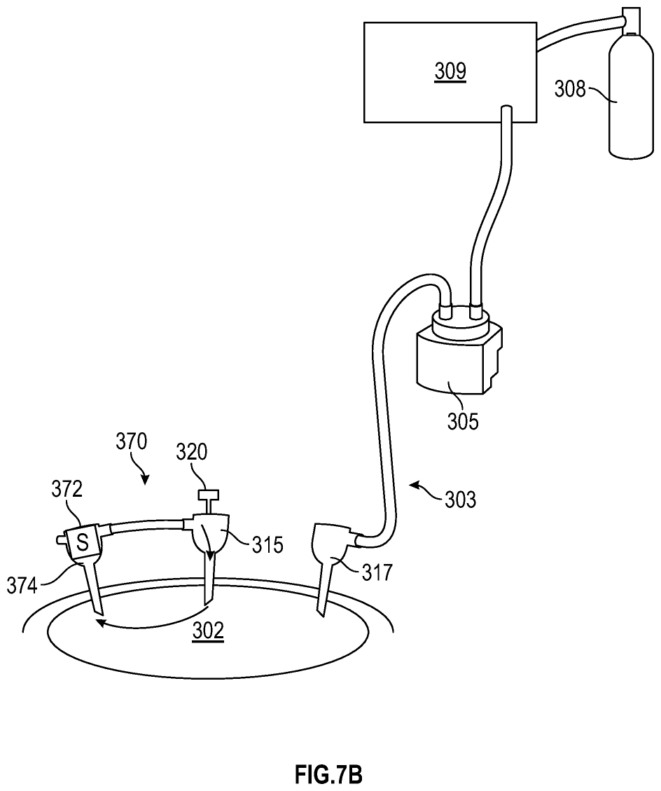

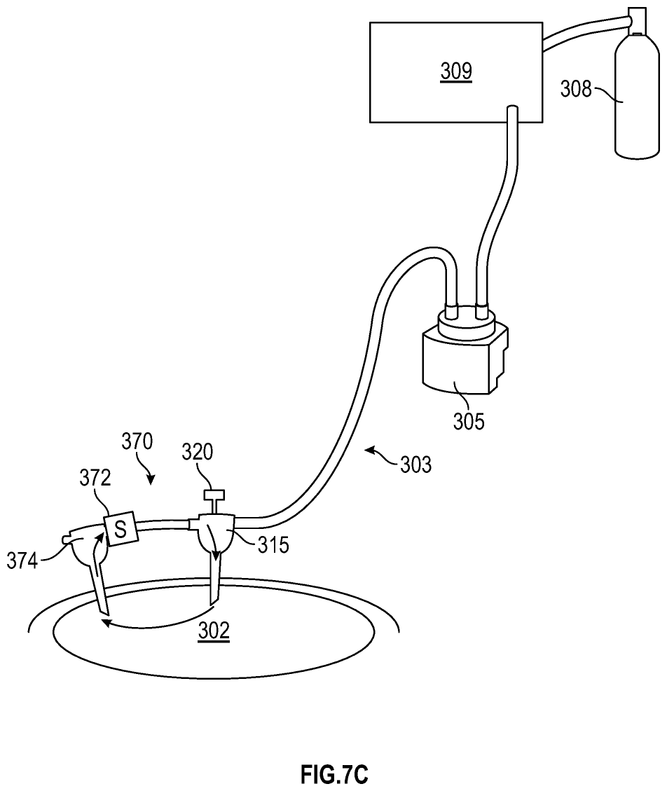

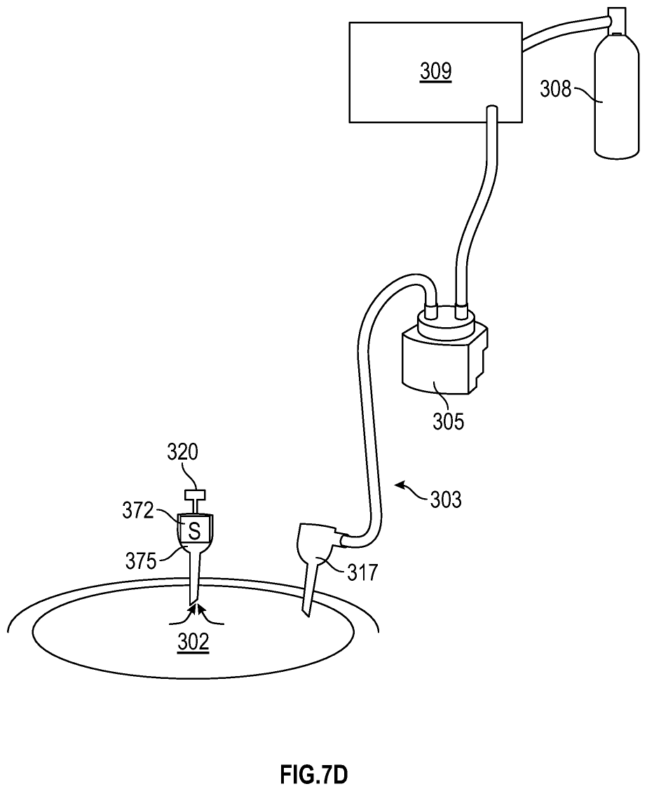

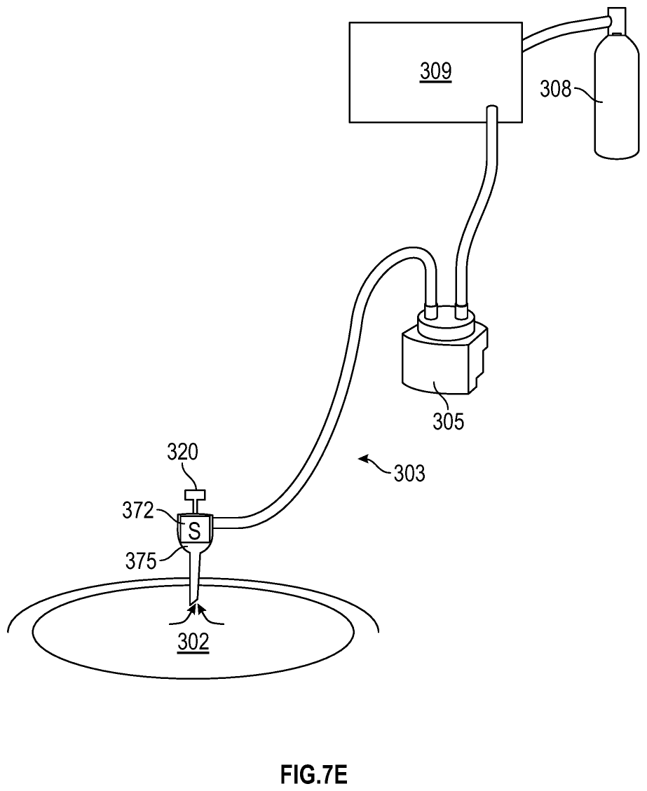

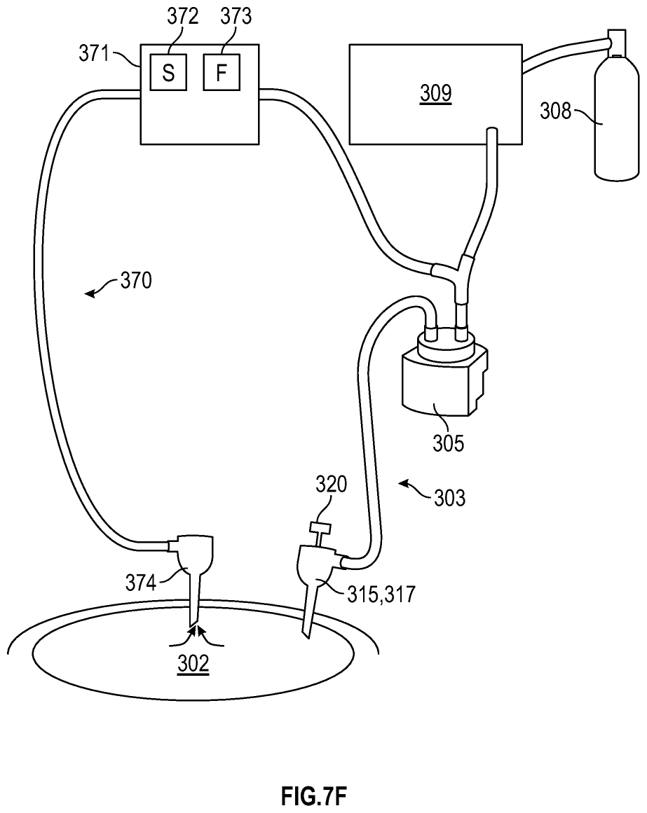

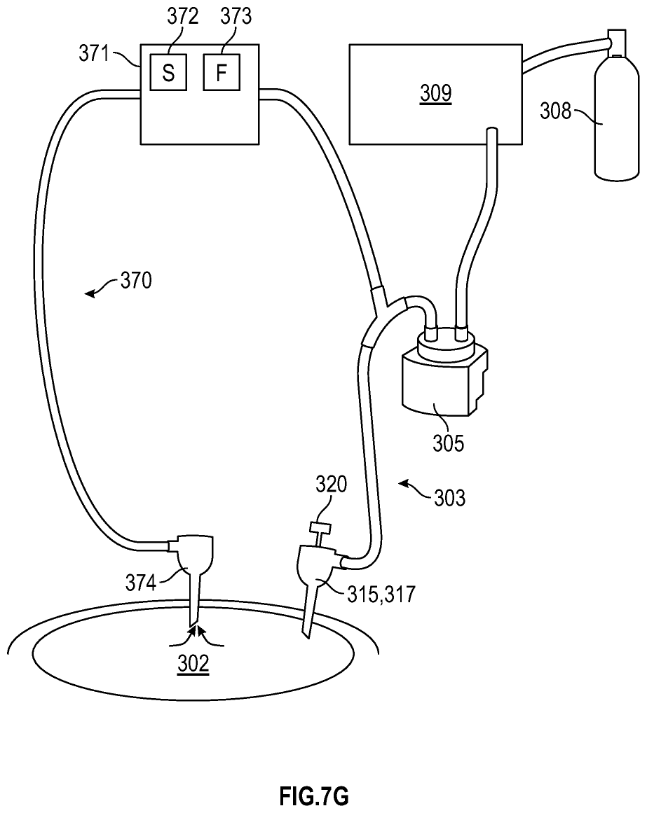

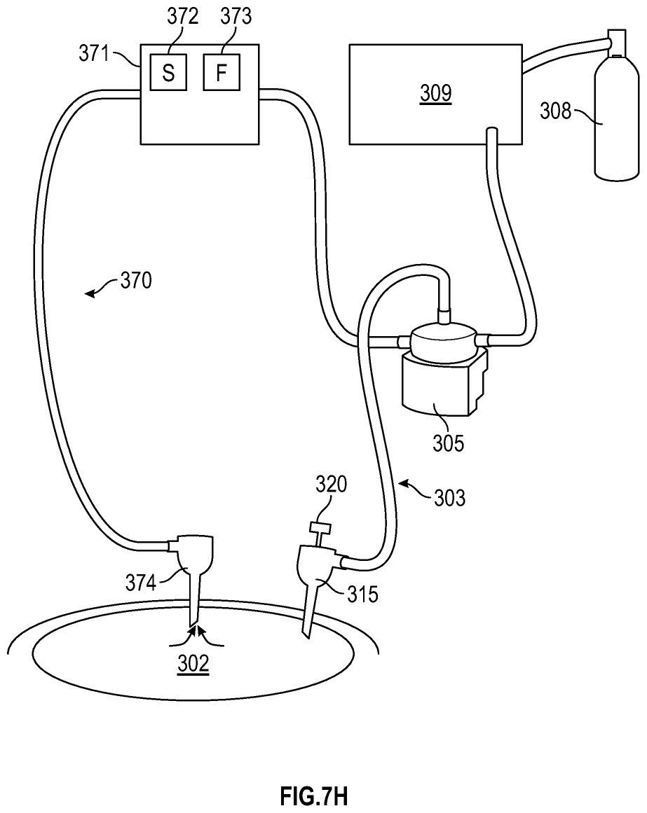

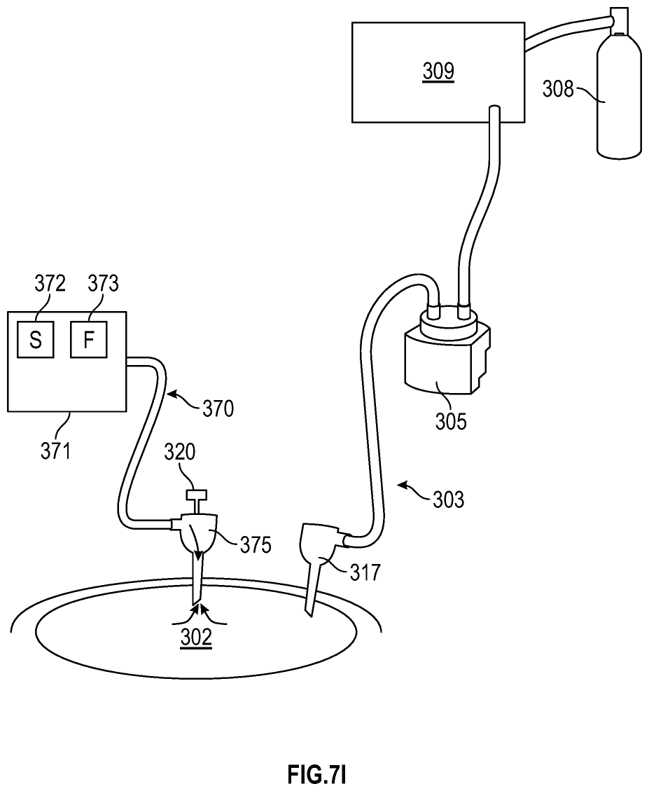

In various embodiments, continuous gas flow through the surgical cannula 315 and/or over the endoscope 320 and/or other surgical tool may be established by a recirculation gas from inside the body cavity 302. The gas may be recirculated through the surgical cannula along a recirculation line 370. The recirculation line 370 may constitute a continuous flow line 304. Rather than gas flow through the continuous flow line 304 being driven by a high pressure gas source 308, as described elsewhere herein, gas flow may be driven by a suction unit 372. The suction unit 372 may be electrically operable. In some embodiments, the suction unit 372 may be fan driven and/or may comprise the features of any standard suction device known in the art. The suction unit 372 may comprise a filter 373 (depicted in FIGS. 7F-7H) so that the recirculated gas is filtered, for example from smoke or debris that may inhibit visualization through the endoscope 320. FIGS. 7A-7I schematically illustrate various examples of surgical systems 300 comprising a recirculation line 370.

FIG. 7A depicts a surgical system comprising a surgical cannula 315, an insufflation cannula 317 at the downstream end of an insufflation line 303, and a recirculation cannula 374, each in fluid communication with the body cavity 302 (e.g., inserted into the body cavity). The insufflation line 303 may comprise the same or similar features as described elsewhere herein. The continuous flow line 304 may be established through a recirculation line 370 extending from the recirculation cannula 374 to the surgical cannula 315. The surgical cannula 315 may be operably coupled to the recirculation cannula 374 via one or more conduits (e.g., tubing) establishing a gas flow pathway. In some embodiments, the suction unit 372 may be a discrete component positioned operatively between the recirculation cannula 374 and the surgical cannula 315. In some embodiments, the suction unit 372 may be connected to fluid flow conduits (e.g., tubing) at an input and/or output of the suction unit 372. In some embodiments, the suction unit 372 may be directly coupled to (e.g., removably attached to) the recirculation cannula 374 and/or the surgical cannula 315. In some embodiments, the suction unit 372 may be integral with and/or incorporated into either the surgical cannula 315 or the recirculation cannula 374. FIG. 7B shows an example of the surgical system 300 in which the suction unit 372 is incorporated into the recirculation cannula 374. In some embodiments, the filter may be separate from the suction unit 372 and the distinct filter or one or more additional filters may be incorporated into the recirculation line 370 in any of the same manners as described with respect to the suction unit 372.

The suction unit 372 drives continuous gas flow, drawing in gas from the body cavity 302 through the recirculation cannula 374 and outputting the suctioned gas back into the body cavity 302 through the surgical cannula 315 such that the gas is forced across a surface of the endoscope 320 and/or other medical instrument as described elsewhere herein. In some embodiments, condensation can be at least partially removed from the recirculated gas prior to forcing the gas over the endoscope 320. For instance, one or more conduits interconnecting the recirculation cannula 374 and the surgical cannula 315 may comprise breathable tubing that allows humidification fluid vapor to diffuse through the tubing wall before it condenses, such as Evaqua™ tubing (Fisher & Paykel Healthcare, Auckland, NZ) for example. Prevention or reduction of humidification fluid vapor condensation may improve the optical visibility through the endoscope 320 by preventing condensation on an endoscope 320 lens. In some embodiments, the surgical system 300 may be used without a ventilation or venting device 322. In some embodiments, the surgical system 300 may further comprise a venting device 322, as described elsewhere herein, and/or one or more of the recirculation cannula 374, the surgical cannula 315, or the insufflation cannula 317 may comprise pressure release features, as described elsewhere herein. In some embodiments, the suction unit 372 may be configured to relieve some pressure from within the body cavity 302.

FIG. 7C illustrates an example of a surgical system 300 in which non-continuous flow from the insufflation line 303 is directed through the surgical cannula 315 such that the continuous flow from the recirculation line 370 and the non-continuous flow are combined within the surgical cannula 315, as described elsewhere herein.

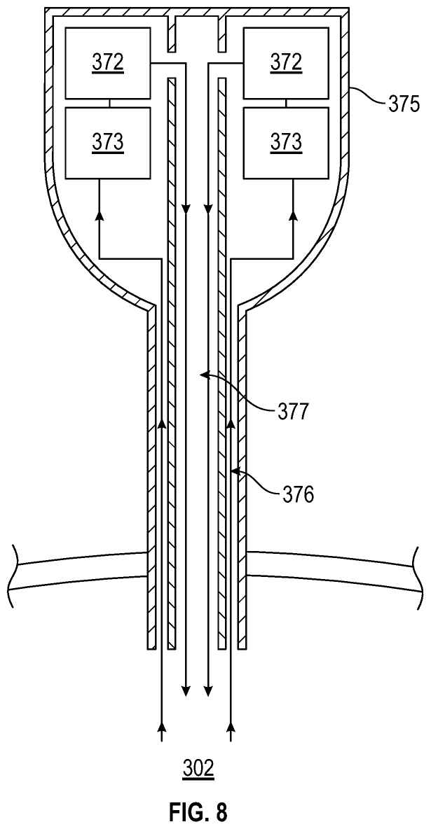

FIG. 7D illustrates an example of a surgical system 300 in which the functions of the surgical cannula 315 and the recirculation cannula 374 are combined into a single cannula 375. The combined surgical recirculation cannula 375 comprises a recirculation flow path 370 comprising a suction unit 372 within the body of the cannula 375. Examples of a combined surgical recirculation cannula 375 are described elsewhere herein (e.g., FIG. 8). The combined recirculation surgical cannula 375 draws gas in through one lumen in fluid communication with the body cavity 302 and forces the gas through another lumen in fluid communication with the body cavity 302 which is configured to receive the endoscope 320 and/or other surgical tools. The combined recirculation surgical cannula 375 may be configured to filter the gas and/or remove humidity in order to reduce and/or prevent condensation as described elsewhere herein. For example, an internal lumen of the combined recirculation surgical cannula 375 may be configured to allow diffusion of humidification fluid vapor to an ambient environment.

FIG. 7E illustrates an example of a surgical system 300 in which non-continuous flow from the insufflation line 303 is directed through the combined recirculation surgical cannula 375 such that the continuous flow from the recirculation line 370 and the non-continuous flow are combined within the cannula 375, similar to FIG. 7C.

FIGS. 7F-7H illustrate examples of surgical systems 300 comprising recirculation lines 370 that converge with downstream portions of an insufflation line 303. Both the insufflation line 303 and the recirculation line 370 end at a surgical cannula 315 which may be separate and distinct from a recirculation cannula 374 or may be the same cannula (not shown). In embodiments in which the insufflation line 303 comprises a humidifier 305, the recirculation line 370 may converge with the insufflation line 303 upstream of the humidifier 305 as shown in FIG. 7F, downstream of the humidifier 305 as shown in FIG. 7G, or at the humidifier 305 as shown in FIG. 7H (similar to the arrangement shown in FIG. 3J).

FIG. 7I illustrates an example of a surgical system 300 comprising a combined recirculation surgical cannula 375. The combined recirculation surgical cannula 375 may be similar to that described with respect to FIGS. 7D and 7E except that the suction unit 372 and/or filter 373 may not be integral with the cannula 375. As shown in FIG. 7I the suction unit 372 and filter 373 may be part of a remote recirculation unit 371. The remote recirculation unit 371 may be coupled to the combined recirculation surgical cannula 375 via one or more conduits. One fluid channel may extend from the cannula 375 to the recirculation unit 371 for venting gas from the body cavity 302 and another fluid channel may extend from the recirculation unit 371 to the cannula 375 for returning gas to the cannula 375 forming a closed loop or circuit with the cannula 375 to provide for continuous flow over the endoscope 370 and/or other medical instruments. The exiting and return channels may be formed within a single conduit (e.g., concentrically or in a parallel fashion) or may be provided for in distinct conduits which may optionally be coupled to one another. The combined recirculation surgical cannula 315 may comprise separate lumens each having a port or other connection site configured to be positioned outside the body cavity for coupling to the one or more conduits. The coupling of the recirculation unit 371 and the conduits to the cannula 375 may form a continuous flow path through the internal lumens of the cannula 375.

FIG. 8 schematically depicts a cross-section of a combined recirculation surgical cannula 375 as described with respect to FIGS. 7D and 7E. As shown in FIG. 8, the cannula 375 may comprise an intake lumen 376 and an output lumen 377 each having an opening configured to be placed in fluid communication with the body cavity 305. As shown in FIG. 8, the intake lumen 376 may be formed concentrically around the output lumen 377. In some embodiments, the lumens 376, 377 may be arranged in a parallel manner. The output lumen 377 may comprise a linear pathway configured to receive an endoscope 320 and/or other medical instruments (not shown). The intake lumen 376 and the output lumen 377 may be operably separated by a suction unit 372 and optionally a filter 373. The filter 373 may be positioned upstream and/or downstream of the suction unit 372. The suction unit 372 and the filter 373 may be positioned in a head portion of the cannula 375 configured to sit outside the body cavity 302. The intake lumen 376 and the output lumen 377 may be joined through the suction unit 372 and optionally the filter 377 to form a continuous gas flow pathway defining the recirculation line 370 or continuous flow line 304. In some embodiments, the suction unit 372 and/or the filter 373 may be disposed remotely to the cannula 375 as described with respect to FIG. 7I. The intake lumen 376 and the output lumen 377 may each comprise a second opening configured to be fluidly coupled to one or more conduits in fluid communication with the suction unit 372 and/or the filter 373. In some embodiments, the second openings may be positioned adjacent one another, may be positioned on opposite sides of the cannula 375, or may be arranged concentrically to couple to a coaxial conduit comprising concentric channels, as described elsewhere herein.

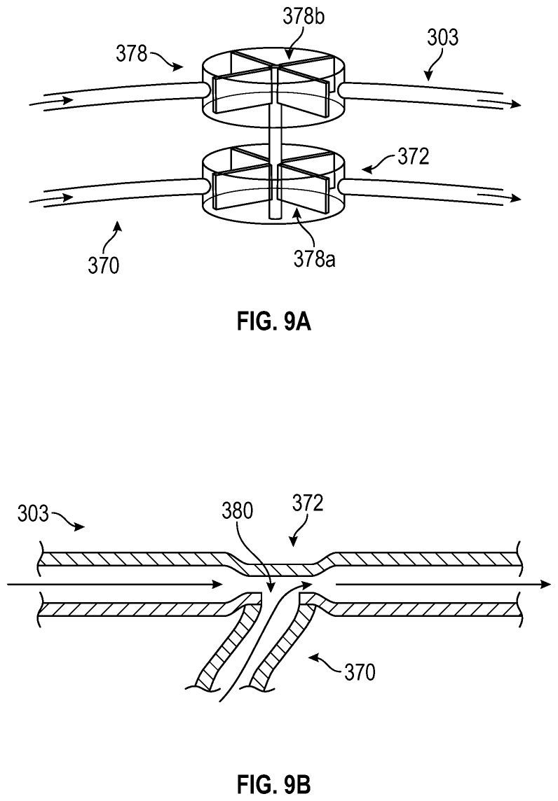

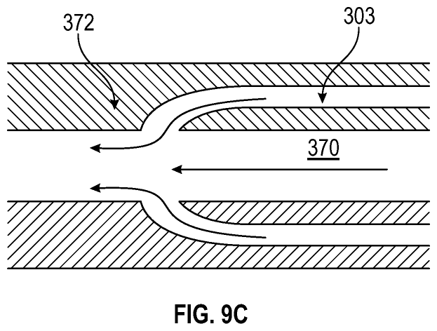

FIGS. 9A-9C schematically illustrate alternative mechanisms for driving the suction unit 372. FIG. 9A illustrates a suction unit 372 which is driven by a turbine-driven fan unit 378. The fan unit 378 may comprise a first fan 378a positioned within the gas flow pathway of the recirculation line 370 and a second fan or turbine 378b positioned outside of the gas flow pathway of the recirculation line 370. The first and second fans 378a, 378b may be operatively coupled by a spindle such that the first and second fans 378a, 378b are forced to spin simultaneously. In some embodiments, the spindle may comprise gearing mechanisms (not shown) which allow the first and second fans 378a, 378b to spin at different speeds. The first and second fans 378a, 378b may be identical or may comprise different configurations (e.g., blade number, blade length, curvatures, etc.). Rotation of the first fan 378a may be configured to provide suction to the recirculation line 370. Rotation of the first fan 378a may be driven by rotation of the second fan 378b. The second fan 378b may be configured as a turbine. Rotation of the turbine 378b may be driven by a flow of gas through a gas line and across the turbine 378b. The flow of gas may be driven by a high pressure gas source. In some embodiments, the gas source may be the gas source 308 used to drive the non-continuous flow through the insufflation line 303. In some embodiments, the gas line may comprise a pressure regulator 342 upstream of the turbine 378b and/or a flow control 344. The downstream end of the gas line may open into the ambient environment. In some embodiments, the second gas line may be the insufflation line 303. The flow of gas driving the turbine 378b may be non-continuous (e.g., pulsatile). A fluid seal may be formed around the spindle to prevent or inhibit gas from escaping (e.g., leaking) from either one or both of the gas flow lines.

FIG. 9B illustrates another example of an alternative mechanism for driving the suction unit 372. The suction unit 372 may comprise a second gas flow line that converges with the recirculation line 370. The second gas flow line may be driven by a pressurized gas source. The pressurized gas source may be the gas source 308 driving the insufflation line 303. In some embodiments, the second gas flow line may be the insufflation line 303, as depicted in FIG. 9B. The gas flow through the portion of the second gas flow line upstream of the convergence may be non-continuous (e.g., pulsatile). The recirculation line 370 may converge with the second gas flow line along a section 380 having a relatively reduced diameter. The reduced diameter section 380 may comprise relatively lower pressure of flowing gas than the upstream portion of the second gas line and/or the downstream portion of the second gas line due to the Venturi effect, similar to that described with respect to FIG. 6D. The reduced pressure within the reduced diameter section 380 may create a pressure gradient between the reduced diameter section 380 and the upstream portion of the recirculation line 370 which may suction gas into the reduced diameter section 380 and the downstream portions of the recirculation line and the second gas flow line (e.g., the insufflation line 303). The suction effect may continue to drive flow through the recirculation line 370 even during off phases of the non-continuous (e.g., pulsatile) flow of the insufflation line 303.

FIG. 9C illustrates a variation of the suction unit 372 described with respect to FIG. 9B. A second gas line comprising high speed gas flow (e.g., the insufflation line 303) may converge with a downstream portion of the recirculation line 370. The high speed flow entering the downstream portion of the recirculation line 370 from the second gas line (e.g., insufflation line 303) may cause a pressure differential which suctions gas into the downstream portion of the recirculation line 370 from the upstream portion of the recirculation line 370. The cross-sectional area of the combined flow paths may be greatest at a point where the gas lines converge, as shown in FIG. 9C, which may cause an isolated area of increased pressure at the convergence. The increased pressure may create a pressure gradient with a downstream portion of the converged gas lines driving gas flow downstream. The convergence may function as a suction unit 372. In some embodiments, the fluid channel or channels of the second gas line may be at least somewhat angled to direct gas flow toward the downstream direction of the recirculation line 370. In some embodiments, at least a portion of the recirculation line 370 may be formed within a same conduit as at least a portion of the second gas flow line (e.g., the insufflation line 303). The upstream portion of the second gas line, for example, may be formed concentrically around an upstream portion of the recirculation line 370, as illustrated in FIG. 9C. In other embodiments, the convergence may be formed by separate conduits that are coupled together.

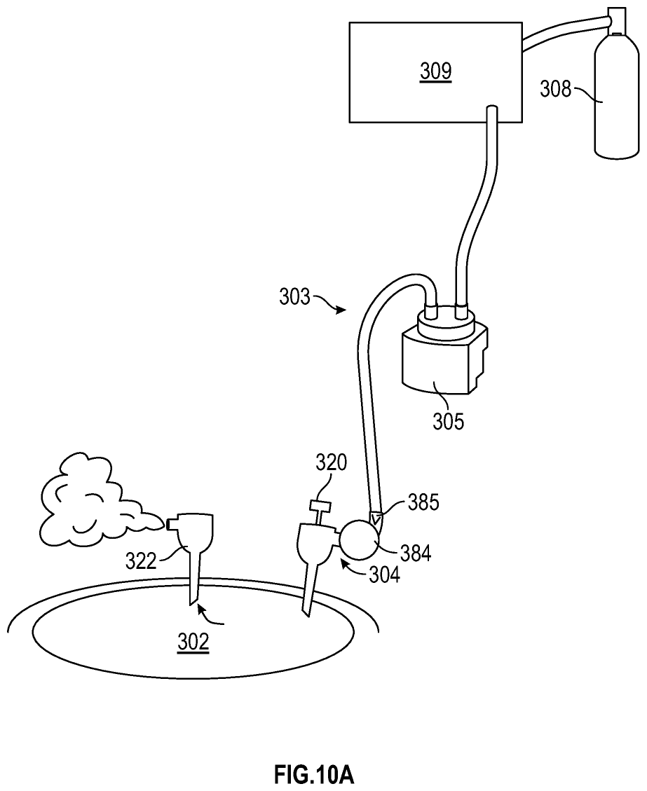

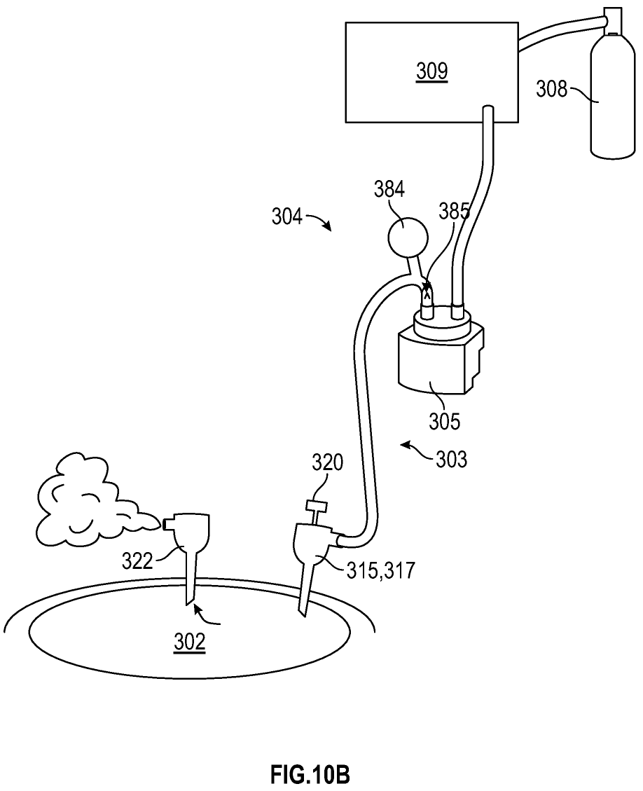

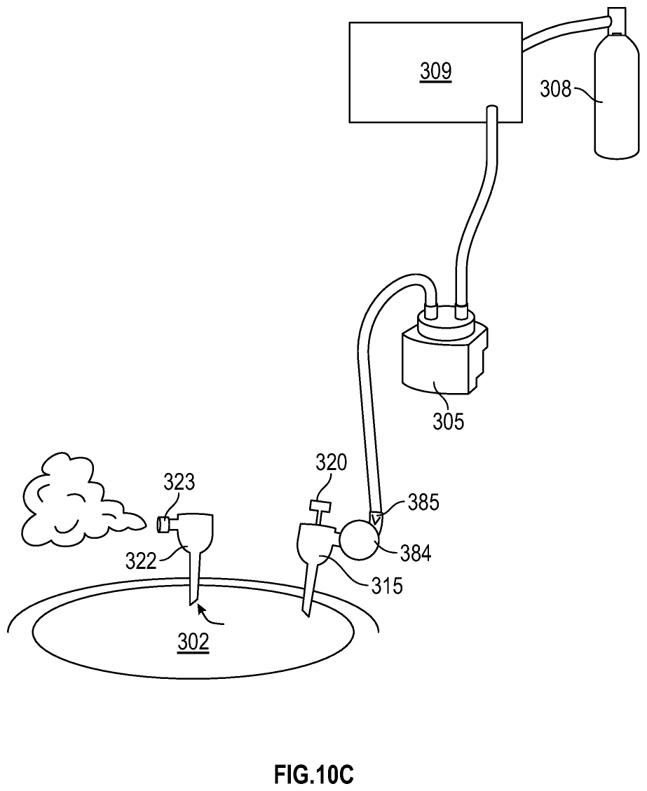

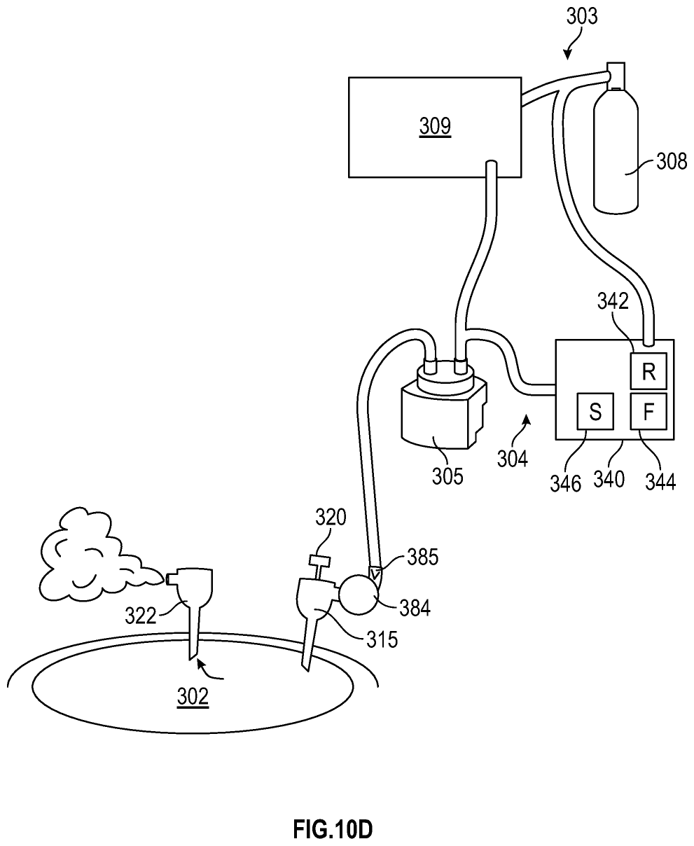

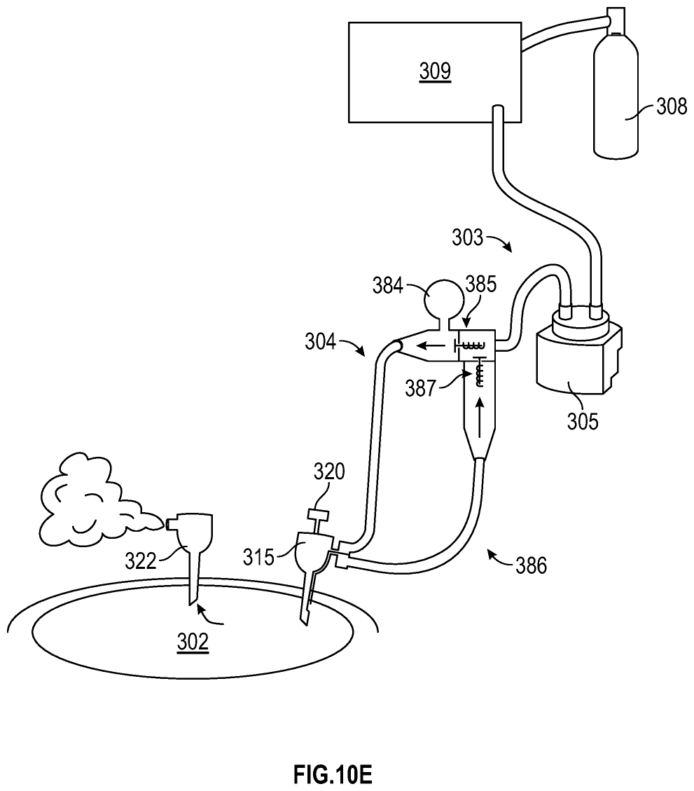

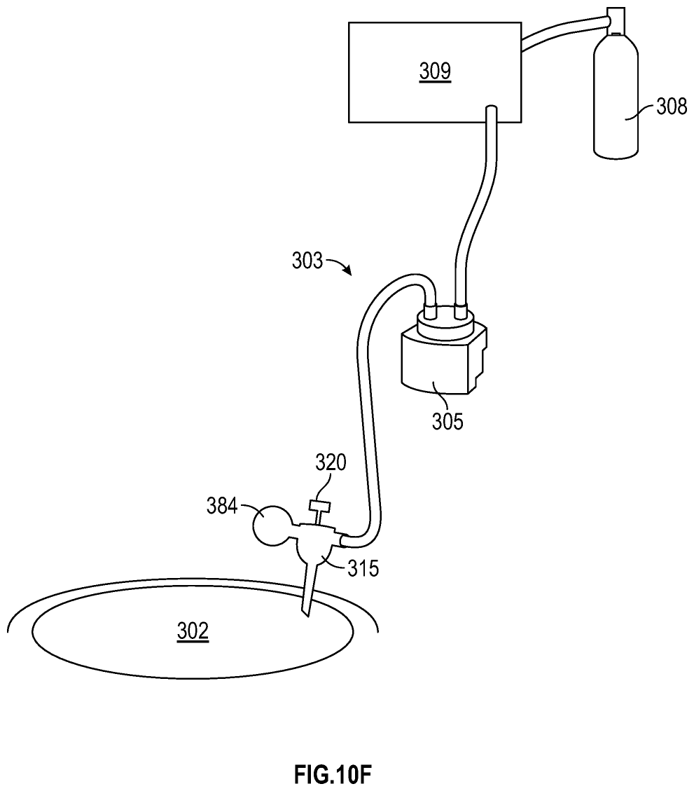

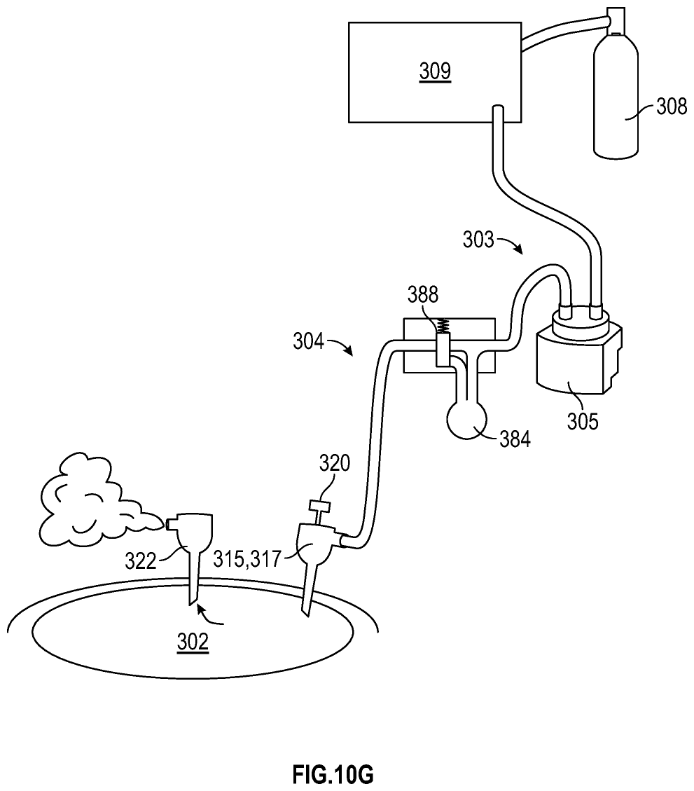

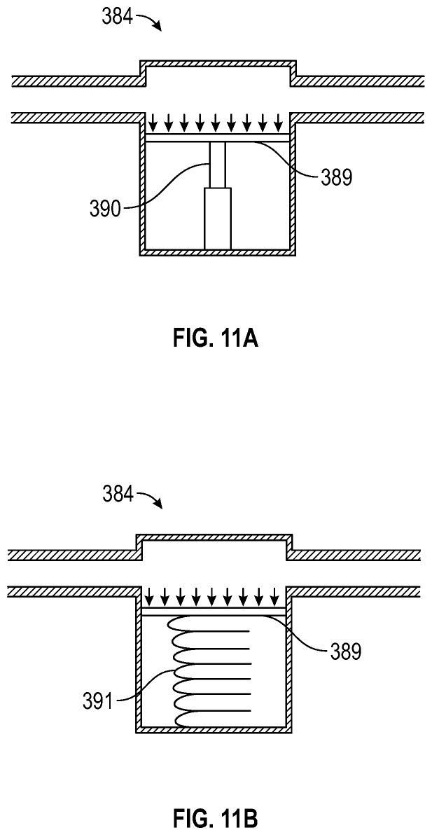

FIGS. 10A-10G schematically illustrate various examples of surgical systems 300 comprising an accumulator 384 for storing and releasing gas to provide a continuous flow over an endoscope 320 and/or other surgical tools. The accumulator 384 may comprise an expandable component configured to create a gas storage space having an expandable volume. In some embodiments, the accumulator 384 may comprise a flexible membrane substantially enclosing the expandable volume. The flexible membrane may be configured to expand or contract/collapse in response to the pressure within the expandable volume. In some embodiments, the accumulator 384 may be or comprise features similar to an inflatable balloon. In other embodiments, the accumulator 384 may comprise alternative configurations (e.g., FIGS. 11A, 11B) which do not rely upon a flexible membrane.

FIGS. 10A-10D schematically illustrate examples of surgical systems 300 comprising an accumulator 384 in line with the insufflation line 303. The accumulator 384 may be configured to expand to store a portion of the gas provided during positive phases or pulses of the non-continuous gas flow supplied via the insufflation line 303. The accumulator 384 may be configured to contract and release at least a portion of the stored gas to the surgical cannula 315 during off phases of the non-continuous gas flow. Between the gas flow provided from the positive phases of the insufflation gas flow and gas flow provided from the release of stored gas from the accumulator 384 during off phases of the insufflation gas flow, a continuous flow of gas may be provided to the surgical cannula 315. Accordingly, a downstream portion of the insufflation line 303 between the accumulator 384 and the surgical cannula 315 may comprise the continuous flow line 304. In the various embodiments described herein, the accumulator 384 may be configured to provide sufficient resistance to expansion such that at least a portion of the gas flow from the insufflation line 303 continues to the body cavity 302 and at least a portion expands and is stored by the accumulator 384.

The accumulator 384 may be positioned operably between a humidifier 305 and the surgical cannula 315, as shown in FIGS. 10A-10D, such that the accumulator 384 stores humidified gas. In some embodiments, the accumulator 384 may comprise an inlet and a separate outlet (not shown in FIG. 10B) such that gas enters the accumulator through the inlet and exits the accumulator through the outlet. The accumulator 384 may comprise or be coupled to one-way flow accumulator valve 385 at the inlet to prevent stored gas from exiting the inlet (e.g., during off phases of the insufflation flow) so that the stored gas is forced to flow through the outlet in a downstream direction. In some embodiments, the accumulator valve 384 may be decoupled from the accumulator 384, but positioned in-line with and upstream of the accumulator 384 (e.g., between a humidifier 305 and the accumulator 384) at a position that does not allow significant upstream travel of the gas released from the accumulator 384. In embodiments in which the accumulator valve 385 is not positioned within the path of the continuous flow line 304 (between the accumulator 384 and the downstream portion gas flow pathway), the accumulator 384 may comprise one port that serves as an inlet and an outlet.

FIG. 10A depicts an example in which an outlet of the accumulator 384 is coupled to an inlet of the surgical cannula 315. FIG. 10B depicts an example in which the accumulator 384 comprises a combined inlet/outlet which is coupled to an outlet of the humidifier 305, for example, via a Y-shaped tubing connector. The accumulator valve 385 may be coupled between the Y-shaped connector and the humidifier 305 to prevent stored gas from being released back into the humidifier 305, as described elsewhere herein. In other embodiments, the accumulator 384 may comprise a separate outlet connected to the downstream portion of the insufflation line 303 and the inlet may be coupled directly to the outlet of the humidifier 305 (not shown). An accumulator valve 385 may be incorporated into the accumulator 384 or positioned between the outlet of the humidifier 305 and the inlet of the accumulator 384. FIG. 10C shows substantially the same surgical system 300 as illustrated in FIG. 10C, but includes an adjustable venting device 322 comprising an adjustable valve 323 or other mechanism which may modulate the release of gas from the body cavity 302 through the cannula 322. The venting device 322 may be the same or similar to that described with respect to FIG. 3L and may allow modulation of the pressure within the body cavity 302.

FIG. 10D depicts a surgical system 300 that is substantially the same as that depicted in FIG. 3G but which includes an accumulator 384 in a converged portion of the insufflation line 303 and continuous flow line 304. The accumulator 384 may be coupled to an inlet of the surgical cannula 315 as shown in FIG. 10A. Continuous flow may be maintained both by direct uninterrupted flow from the gas source 308 through the continuous flow line 304 as well as from gas released from the accumulator 384. The incorporation of the continuous flow line 304 originating from the pressurized gas source 308 with the accumulator 384 may advantageously provide a smoother response from the accumulator (e.g., less peak-to-peak variation in the flow rate through the surgical cannula 315). The continuous flow through the accumulator 384 may dampen the amplitude of volume changes within the expandable volume of the accumulator 384. The addition of the accumulator 384 may compensate for at least some of the reduced flow lost during off phases of the non-continuous insufflation flow.

The incorporation of the accumulator valve 385 in-line with the insufflation line 303 may interfere with pressure sensing within the body cavity 302 and/or downstream portions of the insufflation line 303 by the insufflator 309 and/or humidifier 305. In some embodiments, the surgical system 300 may comprise a feedback line 386, as shown in FIG. 10E. The feedback line 386 may extend from a feedback cannula in fluid communication with the body cavity 302 to a portion of the insufflation line 303 upstream of the accumulator valve 385. In some embodiments, as show in FIG. 10E, the surgical cannula 315 may serve as the feedback cannula. The surgical cannula 315 may comprise a lumen having a separate opening from that configured to receive the endoscope 320 so that the feedback line 386 does not interfere with the flow of gas over the endoscope 320 and/or other medical instruments. The feedback line 386 may comprise a one-way feedback valve 387 positioned where the feedback line 386 meets the insufflation line 303 or some at some other point between the body cavity 302 and the insufflation line 303. As shown in FIG. 10E, the feedback valve 387 may be oriented in an opposite orientation than the accumulator valve 385 with respect to the direction of flow through the insufflation line 303. Upstream pressure from the pressurized gas source 308 flowing through the upstream portion of the insufflation line 303 during positive phases of pressure may be configured to open the accumulator valve 385 and close the feedback valve 387. Any pressure sensors (or other sensors) disposed in an upstream portion of the insufflation line may be able to measure pressure of the body cavity 302 through the insufflation line 303 when the accumulator valve 385 is opened. During off phases of the insufflation flow, the natural biasing of the accumulator valve 385 may cause the accumulator valve 385 to close and the natural biasing of the feedback valve 387 may cause the feedback valve 387 to open. Additionally or alternatively, a higher pressure in the body cavity 302 than an upstream portion of the insufflation line 303 may cause the accumulator valve 385 to close and the feedback valve 387 to open. Any pressure sensors (or other sensors) disposed in an upstream portion of the insufflation line 303 may be able to measure pressure of the body cavity 302 through the insufflation line 303 when the accumulator valve 385 is opened. Incorporation of the feedback line 386 may allow the upstream portion of the insufflation line 303 to always be in fluid communication with the body cavity 302 so that routine sensing functions may continue during the off phase of the insufflation flow.

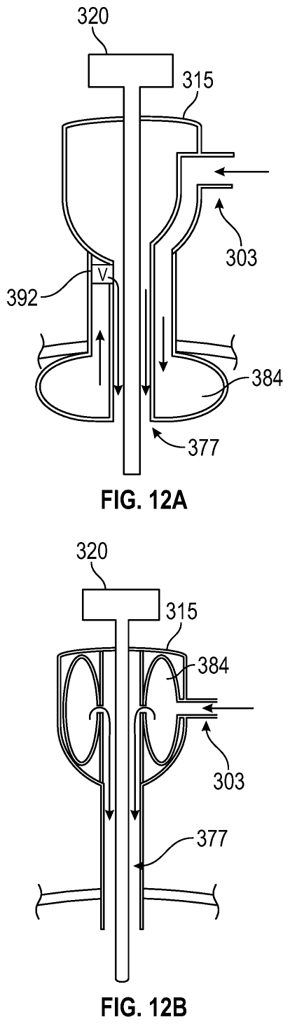

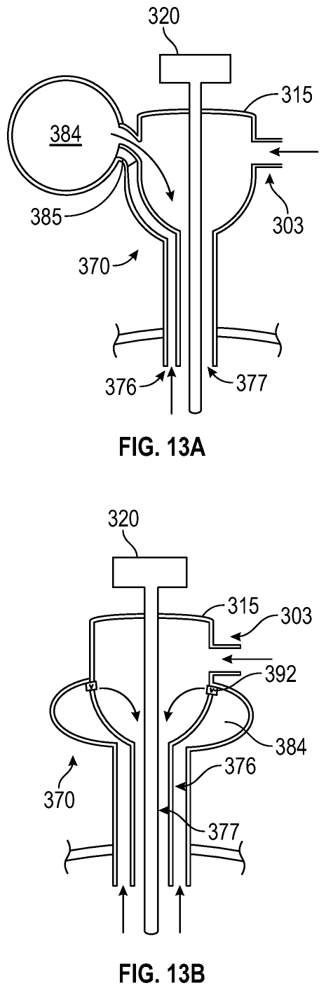

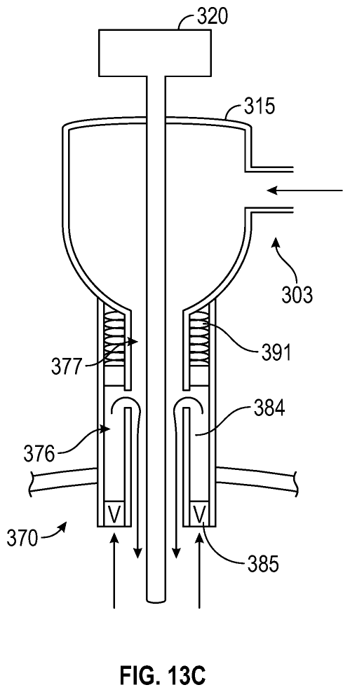

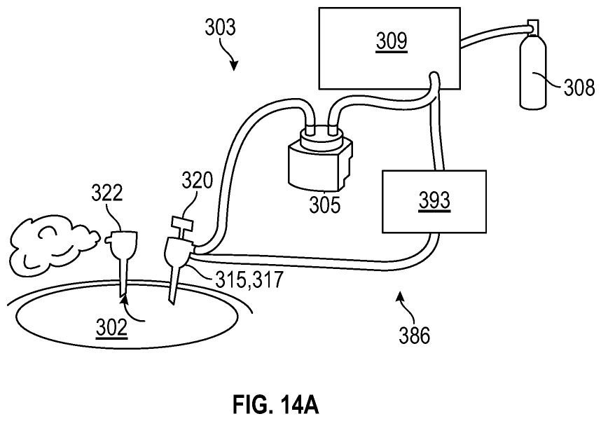

FIG. 10F depicts an example of a surgical system in which both the accumulator 384 and a conduit of the insufflation line 303 are directly and separately coupled to the surgical cannula 315. In some embodiments, the flow path of the insufflation line 303 may continue internally through the surgical cannula 315 and through the accumulator 384 before reaching the endoscope 320 (e.g., FIGS. 12A and 12B), such that the arrangement of the gas flow pathway components is operably similar to that depicted in FIG. 10A. In other embodiments, the accumulator 384 may not be in-line with the insufflation line 303 but may rather be in-line with a recirculation line 370 (e.g., FIGS. 13A-13C). The accumulator 384 may be configured to store vented gas from the body cavity 302 and release it back into the cannula 315 upstream of where the gas flows over the endoscope 320 to provide a continuous gas flow.

FIG. 10G depicts an example of a surgical system 300 in which the accumulator 384 is positioned in parallel with a portion of the insufflation line 303 rather than in-line or in-series. The surgical system 300 may not comprise the one-way accumulator valve 385 positioned operably upstream of the accumulator 384, but may rather comprise a 2/2 accumulator valve switch 388 positioned operably downstream of the accumulator 384. The accumulator valve switch 388 may be configured to switch gas flow to a downstream portion of the insufflation line 303 between gas flow from an outlet of the accumulator 384 and gas flow from a portion of the insufflation line 303 in parallel with the accumulator 384. The accumulator valve switch 388 may be configured to be open to gas flow from the outlet of the accumulator 384 and closed to gas flow from the parallel portion of the insufflation line 303 during off phases of the insufflation flow. The accumulator valve switch 388 may be configured to be closed to gas flow from the outlet of the accumulator 384 and open to gas flow from the parallel portion of the insufflation line 303 during positive phases of the insufflation flow. The parallel arrangement of the accumulator 384 allows for pressure reading and/or other sensing of the body cavity 302 via an upstream component of the insufflation line 303 without the need for a separate feedback line since one of the two parallel lines will always be in open fluid communication with the body cavity 302.

FIGS. 11A and 11B illustrate alternative examples of accumulators 384 which do not necessarily comprise flexible membranes. The accumulators 384 depicted in FIGS. 11A and 11B comprise bodies having fixed total internal volumes which may be adjustably portioned between a volume not in-line with the insufflation flow and an expandable volume that is in-line with the insufflation flow to store gas as described elsewhere herein. A sealing element 389 may separate the two volumes and fluidly seal the volumes from one another. The sealing element 389 may be biased to place the expandable volume in a relatively unexpanded configuration by a compressible piston 390 as shown in FIG. 11A, by a compressible spring 391 as shown in FIG. 11B, or any other suitable biasing element. Increasing the pressure within the expandable volume via gas flow from the insufflation line 303 during positive phases of gas flow may cause the expandable volume to increase so that gas may be stored. When the pressure is relieved, the biasing element may compress the expandable volume forcing the stored gas to exit the accumulator 384. Either of these configurations may be used for any of the accumulators 384 of the surgical systems 300 described herein.

FIGS. 12A and 12B, schematically depict examples of surgical cannulas 315 which incorporate an accumulator 384 in-line with the insufflation line 303 as described elsewhere herein. As shown in FIGS. 12A and 12B, insufflation flow may flow into the body of the surgical cannula 315 from a conduit of the insufflation line 303 and travel into an accumulator 384, such as a flexible accumulator 384 described elsewhere herein for example. The arrangement of the accumulators 384 depicted in FIGS. 12A and 12B may provide a gas flow pathway which is operably similar to that depicted in FIG. 10A. One-way accumulator valves 385 may be positioned upstream of the accumulator 384 (e.g., within a conduit of the insufflation line 303 or attached to an inlet of the surgical cannula 315) as described elsewhere herein. In any of the embodiments described herein, one-way backflow valves 392 may be positioned at downstream ends of the insufflation line to prevent backflow into the accumulator 384 or insufflation line 303 in general. Backflow valves 392 may prevent or inhibit backflow which may be transiently induced via switching of the insufflator 309 from a positive pressure phase to an off-phase and which may interfere with the gas envelope created around the endoscope 320 and/or other surgical tools. In various embodiments, the accumulator 384 may comprise a generally toroidal configuration and be positioned around an output lumen 377 configured to receive the endoscope 320 and/or other surgical tools, as shown in FIGS. 12A and 12B. In some embodiments, the accumulator 384 may be configured to extend outward from a rigid body of the surgical cannula 315 as shown in FIG. 12A. In some embodiments, the accumulator 384 may be positioned within the body cavity 302 as shown in FIG. 12A. The flexible body of the accumulator 384 may be configured to extend laterally outward and, optionally, downward away from a distal or downstream tip of the rigid body of the surgical cannula 315. The accumulator 384 may advantageously be used to form a seal with an incision or natural opening into the body cavity 302. The accumulator 384 may help hold the surgical cannula 315 in a proper position or orientation.

In some embodiments, the accumulator 384 may be entirely contained within a rigid body of the surgical cannula 315 as shown in FIG. 12B. In some embodiments, the accumulator 384 may be disposed in a head portion of the surgical cannula 315 configured to be positioned outside of the body cavity 302 as shown in FIG. 12B.

FIGS. 13A-13C schematically depict examples of surgical cannulas 315 which incorporate an accumulator 384 in-line with a recirculation pathway 370, as described elsewhere herein, to provide continuous gas flow over the endoscope 320 and/or other surgical tools. As shown in FIGS. 13A-13C, the accumulators 384 may be operably positioned between an intake lumen 376 and an output lumen 377, as described elsewhere herein or may be positioned between an intake lumen 376 and the body cavity 302, functionally forming an entrance to the intake lumen 376. One-way accumulator valves 385 preventing upstream release of gas stored in the accumulator 384 and/or one-way backflow valves 387 preventing backflow of gas into the accumulator 384 may be positioned upstream and downstream of the accumulator 384, respectively, even where not explicitly shown. In some implementations, excess pressure from the body cavity 302 may be vented during positive phases of the insufflation flow into the accumulator 384 and released from the accumulator 384 into the output lumen 377 during off-phases of the insufflation flow.

In some embodiments, the accumulator 384 may be configured to extend outward away from a rigid body of the surgical cannula 315. In some embodiments, the accumulator 384 may be configured to be coupled to a lateral side of the surgical cannula 315. The accumulator 384 may extend from a head portion of the surgical cannula 315 as depicted in FIGS. 10F and 12A. In some embodiment, the accumulator 384 may be contained within a rigid body of the surgical cannula 315, such as in a head portion and/or a shaft portion of the cannula 315 configured to extend into the body opening for example. In some embodiments, the accumulator 384 may comprise a generally toroidal configuration and may be positioned concentrically around an output lumen 377 of the surgical cannula configured to receive the endoscope 320 and/or other medical instruments, as shown in FIGS. 13B and 13C. FIG. 13B depicts a flexible accumulator 384 positioned concentrically around the output lumen 377. The outlet of the accumulator 384 may be in direct fluid communication with the output lumen 377 as shown in FIG. 13B. A backflow valve 392 may separate the accumulator 384 and the output lumen 377. FIG. 13C depicts a non-flexible, spring-based accumulator 384, similar to that described with respect to FIG. 11B, disposed around the output lumen 377. A one-way accumulator valve 385 may form an entrance to the accumulator 384 and the recirculation pathway 370.

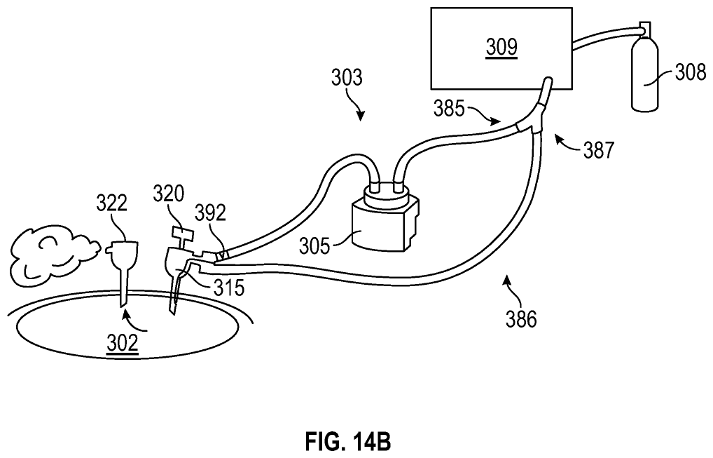

FIGS. 14A and 14B schematically illustrate further embodiments of surgical systems comprising arrangements configured to modify standard surgical systems to provide continuous flow. As described elsewhere herein, any of the various disclosed embodiments of surgical systems 300, may be compatible with standard or commercial insufflators 309, which may be provided separately from one or more of the remaining components of the surgical system 300. In some embodiments, a feedback line 386 may be in fluid communication with the insufflator 309 as described elsewhere herein. The feedback line 386 may be generally arranged and/or comprise components that are the same or similar to those depicted in or described with respect to FIG. 10E. In some embodiments, the feedback line 386 may comprise a feedback modifier 393 as schematically depicted in FIG. 14A. The feedback modifier 393 may modify the pressure detected or sensed within the feedback line 386 (e.g., by the insufflator 309) to be different from the actual pressure of the body cavity 302. For instance, the sensed pressure may be lower than the actual pressure of the body cavity 302, which may trick or manipulate the insufflator 309 into maintaining a positive flow of insufflation gas if, for example, the sensed pressure is below a threshold pressure. The pressure may be modified by any suitable means including those described elsewhere herein, such as with the use of a pressure regulator 342 for example. The downstream end of the feedback line 386 may converge with any downstream portion of the insufflation line 303 (e.g., downstream of the humidifier 303). In some implementations, the downstream end of the feedback line 386 may be coupled (e.g., via a Y-shaped tubing connector) to the inlet of the surgical cannula 315 such that the feedback line 386 can be used with a surgical cannula 315 comprising only a single gas inlet and/or so that additional conduits within the insufflation line 309 are not needed.

FIG. 14B depicts another example of a surgical system 300 comprising a feedback line 386. The surgical system 300 depicted in FIG. 14B may comprise the same or similar features or arrangement of components as depicted in or described with respect to FIG. 10E and/or FIG. 14A. As shown in FIG. 14B, the feedback line 386 may be in fluid communication with a lumen of the surgical cannula 315 that opens into fluid communication with the body cavity 302 on a side of the shaft of the cannula 315, similar to the surgical system 300 shown in FIG. 10E. The positioning of the opening on the side of the shaft of the surgical cannula 315 may advantageously allow the placement of a one-way backflow valve 392 in a downstream portion of the insufflation line 303, such as between the surgical cannula 315 and the conduit configured to couple to the cannula 315 for example. The backflow valve 392 advantageously prevents or inhibits backflow over the endoscope 320 which could interfere with the envelope of continuous gas flow. Accurate pressure readings of the body cavity 302 by the insufflator 309 may be continuously maintained regardless due to the entirely separate lumen provided for in the surgical cannula 315 for coupling to the feedback line 386. The feedback line 386 may or may not comprise a feedback modifier 393 (not shown) as described with respect to FIG. 14A. The arrangement shown in FIG. 14B may advantageously improve a surgical system even where continuous flow is not provided through the surgical cannula 315.

In some embodiments, a first and/or second cannulas as described herein can be configured for directed flow to create a concentric flow around a medical instrument (e.g., a scope such as a laparoscope for example) in order to create a zone of control that can advantageously reduce or prevent smoke, condensation, or other unwanted media from contacting a target section of the medical instrument. In other words, in some cases a gas barrier or envelope, also referred to herein as a gases shroud, gases sheath, protection zone, or region of controlled temperature and humidity, can be created via the directed gas flow cannula, such that gases flow from an opening, through a lumen of the cannula, and through an outlet. This effect can be improved and maximized when combined with continuous air flow to create a continuous concentric flow. In some embodiments, a directed flow cannula can be configured for providing insufflation gases to a surgical cavity and providing a passage for insertion of one or more medical instruments. The cannula can include any number of: a cannula body including an inlet; an elongate shaft extending from the cannula body, the shaft defining a lumen defined by a sidewall, the lumen configured to provide the insufflation gases to the surgical cavity between a gases inlet and an outlet proximate a distal end of the elongate shaft, the lumen in fluid communication with the inlet and the outlet, the lumen also configured to receive a medical instrument therethrough; and/or a guiding element (e.g., ribs or fins) disposed on, within, or around at least a portion of the lumen, the guiding element configured to limit radial movement of the medical instrument within the lumen and prevent the medical instrument from contacting the sidewall of the lumen such that gases flowing into the lumen flow around the medical instrument and create an envelope of insufflation gases that extends distally beyond a distal end of the instrument. The system can also include a venting arrangement. This could be a conventional vent, or the system of this disclosure may be used with venting cannulas or venting attachments as described elsewhere herein.

### Terminology

Examples of medical gases delivery systems and associated components and methods have been described with reference to the figures. The figures show various systems and modules and connections between them. The various modules and systems can be combined in various configurations and connections between the various modules and systems can represent physical or logical links. The representations in the figures have been presented to clearly illustrate the principles and details regarding divisions of modules or systems have been provided for ease of description rather than attempting to delineate separate physical embodiments. The examples and figures are intended to illustrate and not to limit the scope of the present disclosures described herein. For example, the principles herein may be applied to a surgical humidifier as well as other types of humidification systems, including respiratory humidifiers. Examples described herein refer to reducing fogging or condensation on the medical instrument. However, other obstructions to visualization or complications can be prevented or reduced. When reference is made herein to reducing fogging or condensation with the methods, procedures, and devices described herein, it can be understood that these methods, procedures, and devices can also reduce or prevent fogging, condensation, unwanted debris, and/or other field of view obstructions.

As used herein, the term “processor” refers broadly to any suitable device, logical block, module, circuit, or combination of elements for executing instructions. For example, the controller 8 can include any conventional general purpose single- or multi-chip microprocessor such as a Pentium® processor, a MIPS® processor, a Power PC® processor, AMD® processor, ARM® processor, or an ALPHA® processor for example. In addition, the controller 122 can include any conventional special purpose microprocessor such as a digital signal processor or a microcontroller for example. The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein, or can be a pure software in the main processor. For example, logic module can be a software-implemented function block which does not utilize any additional and/or specialized hardware elements. The controller can be implemented as a combination of computing devices, e.g., a combination of a DSP and a microprocessor, a combination of a microcontroller and a microprocessor, a plurality of microprocessors, one or more microprocessors in conjunction with a DSP core, or any other such configuration.

Data storage can refer to electronic circuitry that allows data to be stored and retrieved by a processor. Data storage can refer to external devices or systems, for example, disk drives or solid state drives. Data storage can also refer to fast semiconductor storage (chips), for example, Random Access Memory (RAM) or various forms of Read Only Memory (ROM), which are directly connected to the communication bus or the controller. Other types of data storage include bubble memory and core memory. Data storage can be physical hardware configured to store data in a non-transitory medium.

Although certain embodiments and examples are disclosed herein, inventive subject matter extends beyond the specifically disclosed embodiments to other alternative embodiments and/or uses, and to modifications and equivalents thereof. Thus, the scope of the claims or embodiments appended hereto is not limited by any of the particular embodiments described herein. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is otherwise understood with the context as used in general to convey that an item, term, etc. may be either X, Y or Z. Thus, such conjunctive language is not generally intended to imply that certain embodiments require at least one of X, at least one of Y and at least one of Z each to be present. As used herein, the words “about” or “approximately” can mean a value is within ±10%, within ±5%, or within ±1% of the stated value.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may comprise connected logic units, such as gates and flip-flops, and/or may comprised programmable units, such as programmable gate arrays, application specific integrated circuits, and/or processors. The modules described herein can be implemented as software modules, but also may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with users, operators, other systems, components, programs, and so forth.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

1. A surgical system for delivering gases into a surgical cavity, the surgical system comprising:
a supplementary gases module comprising an inlet configured to be placed in fluid communication with an upstream pressurized gas source and an outlet configured to be placed in fluid communication with a downstream surgical cannula to establish at least one gas flow pathway from the pressurized gas source to the surgical cannula, the supplementary gases module comprising at least one pressure regulator configured to establish a pressure drop between the pressurized gas source and the surgical cannula; and
the surgical cannula, wherein the surgical cannula comprises:
a proximal end configured to be positioned outside of a body cavity and a distal end configured to be inserted into the body cavity,
a medical instrument lumen configured to receive a medical instrument such that the medical instrument extends from an ambient environment outside of the body cavity through the medical instrument lumen into the body cavity, and
an inlet gas flow pathway configured to be placed in fluid communication with the outlet of the supplementary gases module, the inlet gas flow pathway intersecting the medical instrument lumen such that a continuous flow of gas from the pressurized gas source is configured to be flowed over a distal end of the medical instrument when received in the medical instrument lumen.

2. (canceled)

3. (canceled)

4. The surgical system of claim 1, wherein the supplementary gases module comprises one or more sensors configured to sense a parameter of the gas flow downstream of the pressure regulator.

5. The surgical system of claim 4, wherein at least one of the one or more sensors comprises a pressure sensor, a flow rate sensor, a humidity sensor, or a temperature sensor.

6. The surgical system of claim 4, wherein the supplementary gases module is configured to modulate the flow rate of gas through the supplementary gases module in response to a reading determined by at least one of the one or more sensors.

7. The surgical system of claim 1, further comprising a humidifier having an inlet and an outlet positioned operably between the pressure regulator and the surgical cannula, the humidifier being configured to increase the humidity of the continuous gas flow.

8. The surgical system of claim 7, wherein the humidifier is part of the supplementary gases module.

9. The surgical system of claim 7, wherein the inlet of the humidifier is configured to be placed in fluid communication with an outlet of the supplementary gases module and the outlet of the humidifier is configured to be placed in fluid communication with the inlet gas flow pathway of the surgical cannula.

10. The surgical system of claim 1, wherein the supplementary gases module comprises a housing enclosing components of the supplementary gases module.

11. The surgical system of claim 1, wherein the supplementary gases module comprises a gas storage chamber positioned operably downstream of the pressure regulator.

12. The surgical system of claim 11, wherein the supplementary gases module is configured to be positioned vertically above the surgical cannula such that head pressure of gas stored in the gas storage chamber is configured to drive gas flow downstream to the surgical cannula.

13. The surgical system of claim 1, further comprising an insufflator comprising an inlet configured to be placed in fluid communication with the pressurized gas source and an outlet configured to be placed in fluid communication with a downstream insufflation cannula, the insufflator configured to provide a non-continuous flow of gas to the insufflation cannula and to be arranged in parallel with the supplementary gases module between the pressurized gas source and the body cavity.

14. The surgical system of claim 13, wherein the insufflator comprises a pressure regulator configured to establish a pressure drop between the pressurized gas source and the insufflation cannula.

15. The surgical system of claim 13, wherein the insufflator comprises a pressure sensor configured to measure pressure within the body cavity, the insufflator being configured to initiate gas flow to the insufflation cannula or increase the flow rate of gas flow to the insufflation cannula when the measured pressure falls below a predetermined threshold pressure.

16. The surgical system of claim 13, further comprising a humidifier having an inlet and an outlet positioned operably between the insufflator and the insufflation cannula, the humidifier being configured to increase the humidity of the non-continuous gas flow.

17. The surgical system of claim 16, wherein the inlet of the humidifier is configured to be placed in fluid communication with an outlet of the insufflator and/or an outlet of the supplementary gases module.

18.-73. (canceled)

74. The surgical system of claim 1, wherein the surgical cannula comprises at least one heating element configured to heat the surgical cannula and/or the gas flow through the surgical cannula to regulate the temperature of the continuous gas flow.

75. (canceled)

76. (canceled)

77. (canceled)

78. (canceled)

79. A surgical system for delivering gases into a surgical cavity, the insufflation system comprising:
a surgical cannula, wherein the surgical cannula comprises:
a proximal end configured to be positioned outside of a body cavity and a distal end configured to be inserted into the body cavity,
a medical instrument lumen configured to receive a medical instrument such that the medical instrument may extend from an ambient environment outside of the body cavity through the medical instrument lumen into the body cavity,
an inlet gas flow pathway configured to be placed in fluid communication with a pressurized gas source, the inlet gas flow pathway intersecting the medical instrument lumen such that a continuous flow of gas from the pressurized gas source is configured to be flowed over a distal end of the medical instrument when received in the medical instrument lumen, and
a pressure regulator enclosed within the surgical cannula and configured to establish a pressure drop between the pressurized gas source and the medical instrument lumen.

80. (canceled)

81. (canceled)

82. (canceled)

83. A surgical system for delivering gases into a surgical cavity, the insufflation system comprising:
a surgical cannula, wherein the surgical cannula comprises:
a proximal end configured to be positioned outside of a body cavity and a distal end configured to be inserted into the body cavity,
a medical instrument lumen configured to receive a medical instrument such that the medical instrument may extend from an ambient environment outside of the body cavity through the medical instrument lumen into the body cavity,
a recirculation cannula, the recirculation cannula comprising a proximal end configured to be positioned outside of the body cavity, a distal end configured to be inserted into the body cavity, and an intake gas flow pathway configured to allow gas to enter the recirculation cannula from the body cavity, and
a recirculation gas flow pathway connecting the intake gas flow pathway of the recirculation cannula and the medical instrument lumen of the surgical cannula, such that a continuous flow of gas is configured to be flowed over a distal end of the medical instrument when received in the medical instrument lumen.

84. (canceled)

85. (canceled)

86. (canceled)

87. (canceled)

88. (canceled)

89. (canceled)

90. (canceled)

91. (canceled)

92. (canceled)

93. A surgical system for providing a gases flow to a body cavity, the insufflation system comprising:
an insufflator device, a first delivery conduit, and a first cannula,
the first delivery conduit fluidly coupling the insufflator device to the first cannula,
the insufflator device being configured to generate a gases flow of insufflation gases, the insufflation gases being delivered to the first cannula via the first delivery conduit and the insufflation gases being introduced into the body cavity through the first cannula, and
a supplementary gas system configured to deliver an additional flow of gases to the body cavity, the additional flow of gases being in addition to the insufflation gases from the insufflator.

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. A surgical system for providing gases into a body cavity comprising:
the surgical system comprising an insufflator, a first cannula and a conduit, the insufflator delivering gases to the first cannula via the conduit; and
a supplementary gases system configured to provide a supplementary gases flow into the body cavity, the supplementary gases system providing a supplementary gases flow in order to maintain a substantially constant pressure within the body cavity.

109. (canceled)
